(19) United States
(12) Patent Application Publication
(10) Pub. No.: US 2008/0207962 A1
(43) Pub. Date: Aug. 28, 2008

(54) Compositions containing chromium, oxygen, and at least two modifier metals selected the group consisting of gold, silver, and palladium, their preparation, and their use as catalysts and catalyst precursors

(76) Inventors: Velliyur Nott Mallikarjuna Rao, Wilmington, DE (US); Allen Capron Sievert, Elkton, MD (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 12/070,784

(22) Filed: Feb. 21, 2008

Related U.S. Application Data
(60) Provisional application No. 60/903,218, filed on Feb. 23, 2007.
(60) Provisional application No. 60/927,846, filed on May 4, 2007.
(60) Provisional application No. 60/927,848, filed on May 4, 2007.
(60) Provisional application No. 60/927,838, filed on May 4, 2007.
(60) Provisional application No. 60/927,847, filed on May 4, 2007.
(60) Provisional application No. 60/927,839, filed on May 4, 2007.
(60) Provisional application No. 60/927,843, filed on May 4, 2007.
(60) Provisional application No. 60/927,842, filed on May 4, 2007.

(51) Int. Cl. C07C 17/23; B01J 23/26

(52) U.S. Cl. 570/156; 502/313; 502/317; 502/228; 570/123; 570/169

(57) ABSTRACT

A catalyst composition is disclosed that includes chromium, oxygen, and at least two of gold, silver, and palladium as essential constituent elements. The amount of modifier metals (gold, silver, and/or palladium) in the composition is from about 0.05 atom % to about 10 atom % based on the total amount of chromium and modifier metals. Also disclosed is a process for changing the fluorine distribution (i.e., content and/or arrangement) in a hydrocarbon or halogenated hydrocarbon in the presence of the catalyst composition; and methods for preparing said catalyst composition. One preparation method involves (a) co-precipitating a solid by adding ammonium hydroxide (aqueous ammonia) to an aqueous solution of soluble salts of modifier metals and a soluble chromium salt that contains at least three moles of nitrate per mole of chromium in the solution and has a modifier metal content of from about 0.05 atom % to about 10 atom % of the total content of modifier metals and chromium in the solution to form an aqueous mixture containing co-precipitated solid; (b) drying the co-precipitated solid formed in (a); and (c) calcining the dried solid formed in (b) in an atmosphere containing at least 10% oxygen by volume. Another preparation method involves (a) impregnating solid chromium oxide with a solution of a soluble modifier metal salts; (b) drying the impregnated chromium oxide prepared in (a); and optionally; (c) calcining the dried solid. Yet another preparation method involves mixing multiple compositions, each comprising chromium, oxygen, and at least one modifier metal.

This application claims priority of U.S. Patent Application 60/903,218 filed Feb. 23, 2007, and U.S. Patent Applications 60/927,846, 60/927,848, 60/927,838, 60/927,847, 60/927,839, 60/927,843, 60/927,842 filed May 4, 2007.

### FIELD OF THE INVENTION

The present invention relates to catalyst compositions containing chromium, oxygen, and at least two of gold, silver, and palladium. The present invention also relates to the preparation of these catalyst compositions, and their use for the catalytic processing of hydrocarbons and/or halogenated hydrocarbons.

### BACKGROUND OF THE INVENTION

A number of chlorine-containing halocarbons are considered to be detrimental toward the Earth's ozone layer. There is a worldwide effort to develop materials having lower ozone depletion potential and/or lower global warming potential that can serve as effective replacements for these halocarbons. Thus, there is a need for manufacturing processes that provide halogenated hydrocarbons that have lower ozone depletion potential and/or lower global warming potential (e.g., materials that contain less chlorine or no chlorine such as saturated and unsaturated hydrofluorocarbons). The production of hydrofluorocarbons (i.e., compounds containing only carbon, hydrogen and fluorine), has been the subject of considerable interest to provide environmentally desirable products for use as solvents, foam expansion agents, refrigerants, cleaning agents, aerosol propellants, heat transfer media, dielectrics, fire extinguishants and power cycle working fluids. For example, 1,3,3,3-tetrafluoropropene, 2,3,3,3-tetrafluoropropene, 1,1,3,3,3-pentafluoropropene and 1,2,3,3,3-pentafluoropropene have utility in such applications; 1,1,1,3,3-pentafluoropropane has utility as a blowing agent, and 1,1,1,2,3-pentafluoropropane has utility as a refrigerant; 1,1,1,3,3,3-hexafluoropropane and 1,1,1,2,3,3,3-heptafluoropropane have utility as fire extinguishants and 1,1,1,2,3,3-hexafluoropropane has utility as a refrigerant. In addition, these materials can also serve as starting materials and/or intermediates for the production of other fluorinated molecules. Hexafluoropropene is a useful monomer for preparation of fluoropolymers.

Certain metal oxides are used as catalysts and/or catalyst precursors in the manufacture of fluorinated hydrocarbons. Chromium oxide in particular is useful as it has been found that it may be fluorinated by HF at elevated temperature to a give mixture of chromium fluoride and chromium oxyfluoride species which are active catalysts for conversion of C—Cl bonds to C—F bonds in the presence of HF. This conversion of C—Cl bonds to C—F bonds by the action of HF, known generally as halogen exchange, is a key step in many fluorocarbon manufacturing processes.

Chromium oxide compositions useful as catalyst precursors may be prepared in various ways or may take various forms. Chromium oxide suitable for vapor phase fluorination reactions may be prepared by reduction of Cr(VI)trioxide, by dehydration of Guignet's green, or by precipitation of Cr(III) salts with bases (see U.S. Pat. No. 3,258,500). Another useful form of chromium oxide is hexagonal chromium oxide hydroxide with low alkali metal ion content as disclosed in U.S. Pat. No. 3,978,145. Compounds such as MF₄ (M=Ti, Th, Ce), MF₃ (M=Al, Fe, Y), and MF₂ (M=Ca, Mg, Sr, Ba, Zn) have been added to hexagonal chromium oxide hydroxide to increase catalyst life as disclosed in U.S. Pat. No. 3,992,325.

A form of chromium oxide that is a precursor to a particularly active fluorination catalyst is that prepared by pyrolysis of ammonium dichromate as disclosed in U.S. Pat. No. 5,036,036.

The addition of other compounds (e.g., other metal salts) to supported and/or unsupported chromium-based fluorination catalysts has been disclosed. Australian Patent Document No. AU-A-80340/94 discloses bulk or supported catalysts based on chromium oxide (or oxides of chromium) and at least one other catalytically active metal (e.g., Mg, V, Mn, Fe, Co, Ni, or Zn), in which the major part of the oxide(s) is in the crystalline state (and when the catalyst is a bulk catalyst, its specific surface, after activation with HF, is at least 8 m²/g). The crystalline phases disclosed include Cr₂O₃, CrO₂, NiCrO₃, NiCrO₄, NiCr₂O₄, MgCrO₄, ZnCr₂O₄ and mixtures of these oxides. U.S. Patent Application Publication No. US2001/0011061 A1 discloses chromia-based fluorination catalysts (optionally containing Mg, Zn, Co, and Ni) in which the chromia is at least partially crystalline.

Other compositions and preparation methods are disclosed in U.S. Pat. No. 5,494,873, U.S. Patent Application Publication No. US2005/0228202, U.S. Patent Application Publication No. US2005/0227865, and U.S. Patent Application Publication No. US2007/0004585.

There remains a need for catalysts that can be used for processes such as the selective fluorination and chlorofluorination of saturated and unsaturated hydrocarbons, hydrochlorocarbons, hydrochlorofluorocarbons, and chlorofluorocarbons, the fluorination of unsaturated fluorocarbons, the isomerization and disproportionation of fluorinated organic compounds, the dehydrofluorination of hydrofluorocarbons, and the chlorodefluorination of fluorocarbons.

### SUMMARY OF THE INVENTION

This application includes seven different general categories of invention designated below by sections A through G, respectively.

### A.

This invention provides a catalyst composition comprising chromium, oxygen, and at least two modifier metals selected from the group consisting of gold, silver, and palladium as essential constituent elements thereof, wherein the total amount of modifier metals is from about 0.05 atom % to about 10 atom % based on the total amount of chromium and modifier metals in the catalyst composition.

This invention also provides a process for changing the fluorine distribution (i.e., content and/or arrangement) in a hydrocarbon or halogenated hydrocarbon in the presence of a catalyst. The process is characterized by using said catalyst composition of this invention as the catalyst.

This invention also provides a method for preparing said catalyst composition. The method comprises; (a) co-precipitating a solid by adding ammonium hydroxide (aqueous ammonia) to an aqueous solution of soluble modifier metal salts and a soluble chromium salt that contains at least three moles of nitrate (i.e., NO₃⁻) per mole of chromium (i.e., Cr⁺³) in the solution and has a modifier metal content of from about 0.05 atom % to about 10 atom % of the total content of modifier metals and chromium in the solution to form an aqueous mixture containing co-precipitated solid; (b) drying said co-precipitated solid formed in (a); and (c) calcining said dried solid formed in (b) in an atmosphere containing at least 10% oxygen by volume.

This invention further provides another method for preparing said catalyst composition. The method comprises (a) impregnating solid chromium oxide with a solution of a soluble modifier metal salts, (b) drying the impregnated chromium oxide prepared in (a); and optionally, (c) calcining the dried solid.

This invention further provides yet another method for preparing said catalyst composition. The method comprises mixing multiple compositions, each comprising chromium, oxygen, and at least one modifier metal.

### B.

This invention provides a process for making CF₃CH₂CHF₂ (HFC-245fa) and CF₃CHFCH₂F (HFC-245eb). The process comprises (a) reacting hydrogen fluoride (HF), chlorine (Cl₂), and at least one halopropene of the formula CX₃CCl═CClX, wherein each X is independently selected from the group consisting of F and Cl, to produce a product comprising CF₃CCl₂CClF₂ (CFC-215aa) and CF₃CClFCCl₂F (CFC-215bb), wherein said CF₃CCl₂CClF₂ and CF₃CClFCCl₂F are produced in the presence of a catalyst composition comprising chromium, oxygen, and at least two modifier metals selected from the group consisting of gold, silver, and palladium as essential constituent elements, wherein the total amount of modifier metals in said catalyst composition is from about 0.05 atom % to about 10 atom % based on the total amount of chromium and modifier metals in the catalyst composition; (b) reacting CF₃CCl₂CClF₂ and CF₃CClFCCl₂F produced in (a) with hydrogen (H₂), to produce a product comprising CF₃CH₂CHF₂ (HFC-245fa) and CF₃CHFCH₂F (HFC-245eb); and (c) recovering CF₃CH₂CHF₂ and CF₃CHFCH₂F from the product produced in (b).

### C.

This invention provides a process for making at least one compound selected from the group consisting of 1,3,3,3-tetrafluoropropene (CF₃CH═CHF, HFC-1234ze) and 2,3,3,3-tetrafluoropropene (CF₃CF═CH₂, HFC-1234yf). The process comprises (a) reacting hydrogen fluoride (HF), chlorine (Cl₂), and at least one halopropene of the formula CX₃CCl═CClX, wherein each X is independently selected from the group consisting of F and Cl, to produce a product comprising CF₃CCl₂CClF₂ (CFC-215aa) and CF₃CClFCCl₂F (CFC-215bb), wherein said CF₃CCl₂CClF₂ and CF₃CClFCCl₂F are produced in the presence of a catalyst composition comprising chromium, oxygen, and at least two modifier metals selected from the group consisting of gold, silver, and palladium as essential constituent elements, wherein the total amount of modifier metals in said catalyst composition is from about 0.05 atom % to about 10 atom % based on the total amount of chromium and modifier metals in the catalyst composition; (b) reacting CF₃CCl₂CClF₂ and CF₃CClFCCl₂F produced in (a) with hydrogen (H₂) to produce a product comprising CF₃CH₂CHF₂ (HFC-245fa) and CF₃CHFCH₂F (HFC-245eb); (c) dehydrofluorinating CF₃CH₂CHF₂ and CF₃CHFCH₂F produced in (b) to produce a product comprising CF₃CH═CHF (HFC-1234ze) and CF₃CF═CH₂ (HFC-1234yf); and (d) recovering at least one compound selected from the group consisting of CF₃CH═CHF and CF₃CF═CH₂ from the product produced in (c).

### D.

This invention provides a process for the manufacture of 1,1,1,3,3,3-hexafluoropropane (HFC-236fa) and at least one compound selected from the group consisting of 1,1,1,2,3,3-hexafluoropropane (HFC-236ea) and hexafluoropropene (HFP, CF₃CF═CF₂). The process comprises (a) reacting HF, Cl₂, and at least one halopropene of the formula CX₃CCl═CClX; wherein each X is independently selected from the group consisting of F and Cl, to produce a product comprising CF₃CCl₂CF₃ and CF₃CClFCClF₂, wherein said CF₃CCl₂CF₃ and CF₃CClFCClF₂ are produced in the presence of a catalyst composition comprising chromium, oxygen, and at least two modifier metals selected from the group consisting of gold, silver, and palladium as essential constituent elements, wherein the total amount of modifier metals in said catalyst composition is from about 0.05 atom % to about 10 atom % based on the total amount of chromium and modifier metals in the catalyst composition; (b) reacting CF₃CCl₂CF₃ and CF₃CClFCClF₂ produced in (a) with hydrogen, optionally in the presence of HF, to produce a product comprising CF₃CH₂CF₃ and at least one compound selected from the group consisting of CHF₂CHFCF₃ and CF₃CF═CF₂; and (c) recovering from the product produced in (b), CF₃CH₂CF₃ and at least one compound selected from the group consisting of CHF₂CHFCF₃ and CF₃CF═CF₂.

### E.

This invention provides a process for the manufacture of at least one compound selected from the group consisting of 1,1,3,3,3-pentafluoropropene (CF₃CH═CF₂, HFC-1225zc) and 1,2,3,3,3-pentafluoropropene (CF₃CF═CHF, HFC-1225ye). The process comprises (a) reacting HF, Cl₂, and at least one halopropene of the formula CX₃CCl═CClX, wherein each X is independently selected from the group consisting of F and Cl, to produce a product comprising CF₃CCl₂CF₃ and CF₃CClFCClF₂, wherein said CF₃CCl₂CF₃ and CF₃CClFCClF₂ are produced in the presence of a catalyst composition comprising chromium, oxygen, and at least two modifier metals selected from the group consisting of gold, silver, and palladium as essential constituent elements, wherein the total amount of modifier metals in said catalyst composition is from about 0.05 atom % to about 10 atom % based on the total amount of chromium and modifier metals in the catalyst composition; (b) reacting CF₃CCl₂CF₃ and CF₃CClFCClF₂ produced in (a) with hydrogen, optionally in the presence of HF, to produce a product comprising CF₃CH₂CF₃ and CF₃CHFCHF₂; (c) dehydrofluorinating CF₃CH₂CF₃ and CF₃CHFCHF₂ produced in (b) to produce a product comprising CF₃CH═CF₂ and CF₃CF═CHF; and (d) recovering at least one compound selected from the group consisting of CF₃CH═CF₂ and CF₃CF═CHF from the product produced in (c).

### F.

This invention provides a process for making at least one compound selected from 1,1,1,3,3-pentafluoropropane (HFC-245fa) and 1,1,1,3,3,3-hexafluoropropane (HFC-236fa). The process comprises (a) reacting hydrogen fluoride (HF) and at least one halopropene of the formula CX₃CCl═CClX, wherein each X is independently selected from the group consisting of F and Cl, to produce a product comprising at least one compound selected from CF₃CCl═CF₂ (CFC-1215xc) and CF₃CHClCF₃ (HCFC-226da), wherein said CF₃CCl═CF₂ and CF₃CHClCF₃ are produced in the presence of a catalyst composition comprising chromium, oxygen, and at least two modifier metals selected from the group consisting of gold, silver, and palladium as essential constituent elements, wherein the total amount of modifier metals in said catalyst composition is from about 0.05 atom % to about 10 atom % based on the total amount of chromium and modifier metals in the catalyst composition; (b) reacting at least one compound selected from CF₃CCl═CF₂ and CF₃CHClCF₃ produced in (a) with hydrogen (H₂), optionally in the presence of HF, to produce a product comprising at least one compound selected from CF₃CH₂CHF₂ (HFC-245fa) and CF₃CH₂CF₃ (HFC-236fa); and (c) recovering at least one compound selected from CF₃CH₂CHF₂ and CF₃CH₂CF₃ from the product produced in (b).

### G.

This invention provides a process for making at least one compound selected from the group consisting of 1,3,3,3-tetrafluoropropene (CF₃CH═CHF, HFC-1234ze) and 1,1,3,3,3-pentafluoropropene (CF₃CH═CF₂, HFC-1225zc). The process comprises (a) reacting hydrogen fluoride (HF) and at least one halopropene of the formula CX₃CCl═CClX, wherein each X is independently selected from the group consisting of F and Cl, to produce a product comprising at least one compound selected from CF₃CCl═CF₂ (CFC-1215xc) and CF₃CHClCF₃, (HCFC-226da), wherein said CF₃CCl═CF₂ and CF₃CHClCF₃ are produced in the presence of a catalyst composition comprising chromium, oxygen, and at least two modifier metals selected from the group consisting of gold, silver, and palladium as essential constituent elements, wherein the total amount of modifier metals in said catalyst composition is from about 0.05 atom % to about 10 atom % based on the total amount of chromium and modifier metals in the catalyst composition; (b) reacting at least one compound selected from CF₃CCl═CF₂ and CF₃CHClCF₃ produced in (a) with hydrogen (H₂), optionally in the presence of HF, to produce a product comprising at least one compound selected from CF₃CH₂CHF₂ (HFC-245fa) and CF₃CH₂CF₃ (HFC-236fa); (c) dehydrofluorinating at least one compound selected from CF₃CH₂CHF₂ and CF₃CH₂CF₃ produced in (b) to produce a product comprising at least one compound selected from CF₃CH═CHF (HFC-1234ze) and CF₃CH═CF₂ (HFC-1225zc); and (d) recovering at least one compound selected from the group consisting of CF₃CH═CHF and CF₃CH═CF₂ from the product produced in (c).

### DETAILED DESCRIPTION

### A.

Invention Category A of this application includes new catalyst compositions. New catalyst compositions of this invention comprise chromium, oxygen, modifier metals (e.g., modifier metal-containing chromium oxide) and contain from about 0.05 atom % to about 10 atom % modifier metals based on the total amount of modifier metals and chromium in the catalyst composition. Of note are compositions comprising silver and gold wherein the mole ratio of silver to gold is from about 10:1 to about 1:10. Also of note are compositions comprising silver and palladium wherein the mole ratio of silver to palladium is from about 10:1 to about 1:10. Also of note are compositions comprising palladium and gold wherein the mole ratio of palladium to gold is from about 10:1 to about 1:10.

In one embodiment of this invention, the catalyst composition comprises alpha-chromium oxide (i.e., α-Cr₂O₃) and metallic gold (i.e., gold in the zero oxidation state) and metallic silver (i.e., silver in the zero oxidation state). In another embodiment of this invention, the catalyst composition comprises alpha-chromium oxide (i.e., α-Cr₂O₃) and metallic gold (i.e., gold in the zero oxidation state) and palladium. In another embodiment of this invention, the catalyst composition comprises alpha-chromium oxide (i.e., α-Cr₂O₃), metallic gold (i.e., gold in the zero oxidation state), metallic silver (i.e., silver in the zero oxidation state), and palladium. Of note are embodiments wherein at least 50 weight % of the chromium component is present as alpha-chromium oxide. Also of note are embodiments wherein the gold component consists essentially of metallic gold having an average particle size of from about 1 nanometer to about 500 nanometers. In certain embodiments of this invention, particles of metallic gold and metallic silver and/or palladium are dispersed in a matrix comprising chromium oxide. In some embodiments particles of metallic gold and metallic silver and/or palladium are supported on a chromium oxide support.

In other embodiments of this invention, the catalyst composition comprises alpha-chromium oxide (i.e., α-Cr₂O₃), metallic silver (i.e., silver in the zero oxidation state) and palladium. Of note are embodiments wherein at least 50 weight % of the chromium component is present as alpha-chromium oxide. In certain embodiments of this invention, particles of metallic silver and palladium are dispersed in a matrix comprising chromium oxide. In some embodiments particles of metallic silver and palladium are supported on a chromium oxide support.

The catalyst compositions of this invention may further comprise fluorine as an essential constituent element.

The catalyst compositions of the present invention may be prepared by co-precipitation. The catalyst compositions prepared by the co-precipitation processes comprise particles of at least two of metallic gold, metallic silver, and palladium dispersed in a matrix comprising chromium oxide.

In a typical co-precipitation technique, an aqueous solution of soluble modifier metal salts and a soluble chromium salt (e.g. gold(III) and chromium(III) salts) is prepared. The relative amount of modifier metal and chromium salts in the aqueous solution is dictated by the amount of modifier metal relative to chromium desired in the final catalyst composition. Of note is an aqueous solution having a modifier metal content of from about 0.05 atom % to about 10 atom % of the total content of modifier metals and chromium in the solution. The concentration of chromium salt in the aqueous solution is typically from about 0.3 to about 3 molar (moles per liter). Preferred concentration of chromium salt is from about 0.75 to about 1.5 molar. Chromium salts suitable for preparation of the aqueous solution are the nitrate, sulfate, acetate, formate, oxalate, phosphate, bromide, chloride, and various hydrated forms of these salts. Other suitable chromium salts include hexacoordinate complexes of the formula [CrL_{6-z}A_{z}]^{+(3-z)} where each L is a neutral (i.e., uncharged) ligand selected from the group consisting of H₂O, NH₃, C₁-C₄ primary, secondary, and tertiary organic amines, C₁-C₄ alkyl nitrites, and pyridine and its derivatives. Each A is an anionic ligand selected from the group consisting of fluoride, chloride, bromide, iodide, hydroxide, nitrite, and nitrate. Z has a value of from 0 to 3. L can also be neutral bidentate ligands such as ethylene diamine. In such a situation, each neutral bidentate ligand is equivalent to two L ligands since it occupies two coordination sites. A can also be anionic bidentate ligands such as C₁-C₄ carboxylate. In such a situation, each anionic bidentate ligand is equivalent to two A ligands since it occupies two coordination sites. A can also be dianionic ligands such as sulfates. In such a situation, each dianionic ligand is equivalent to two A ligands. Such a dianionic ligand may occupy more than one coordination site.

Chromium(III)nitrate, or a hydrated form such as [Cr(NO₃)₃(H₂O)₉], is the most preferred chromium salt for the preparation of the aqueous solutions for the co-precipitation.

Gold salts suitable for preparation of the aqueous solution include the acetate, bromide, chloride, and various hydrated forms of these salts. Gold(III)chloride and hydrogen tetrachloroaurate (HAuCl₄.3H₂O) are the most preferred gold salts for the preparation of the aqueous solutions for the co-precipitation. Suitable silver salts include silver(I)nitrate. Suitable palladium salts include palladium(II)chloride, tetrachloropalladate salts, and palladium(II)nitrate.

The aqueous solution of the soluble modifier metal salts and soluble chromium salts is then treated with a base such as ammonium hydroxide (aqueous ammonia) to co-precipitate modifier metals and chromium salts as the hydroxides. The addition of ammonium hydroxide to the aqueous solution of modifier metals and chromium salts is typically carried out gradually over a period of 1 to 12 hours. The pH of the solution is monitored during the addition of base. The final pH is typically from about 6.0 to about 10.0, preferably from about 7.5 to about 9.0 and most preferably from about 8.0 to about 8.7. The co-precipitation of the modifier metal hydroxides/chromium hydroxide mixture is typically carried out at a temperature of from about 15° C. to about 60° C., preferably from about 20° C. to about 40° C. After the ammonium hydroxide is added, the mixture is typically stirred for up to 24 hours.

After the co-precipitation of the mixture of modifier metal hydroxides and chromium hydroxide is complete, the co-precipitated solid is dried. In one embodiment of this invention, the co-precipitated solid is dried by evaporation. In another embodiment of this invention, the co-precipitated solid is collected by filtration and washed with deionized water prior to drying.

After the co-precipitated solid has been dried, the solid is then calcined at temperatures of from about 375° C. to about 1000° C., preferably from about 400° C. to about 900° C., and most preferably from about 400° C. to about 600° C. for about 12 to 24 hours. The calcination can be carried out in an atmosphere containing at least 10% oxygen by volume. Preferably, the calcination is carried out in the presence of air.

In one embodiment of this invention, the co-precipitated solid also contains nitrate salts (e.g. when chromium(III)nitrate is used as a soluble chromium salt for the co-precipitation). In such a situation, after the co-precipitated solid has been dried, but before calcination, the nitrate salts contained in the dried co-precipitated solid can be decomposed by carefully heating the solid from about 150° C. to about 350° C.

The catalyst compositions of the present invention may also be prepared by impregnating solid chromium oxide with a solution of soluble modifier metal salts. In this technique, an aqueous solution of soluble modifier metal salts is added with stirring to solid chromium oxide. It is preferable to adjust the total volume of the aqueous modifier metal salt solution so that after addition, the resulting modifier metal salt-impregnated chromium oxide has a minimum amount of excess liquid. The entire modifier metal salt-impregnated chromium oxide, with any excess liquid present, is dried. In one embodiment of this invention, the entire modifier metal salt-impregnated chromium oxide, with any excess liquid present, is dried by evaporation at about 100° C. to 120° C. in air for about 12 hours. The dried solid is then calcinated at about 200° C. to 400° C. for about 12 to 24 hours. The calcination can be carried out in an atmosphere containing at least 10% oxygen by volume. Preferably, the calcination is carried out in the presence of air. The catalyst compositions prepared by such impregnation processes comprise particles of at least two of metallic gold, metallic silver, and palladium supported on a chromium oxide support. The solid chromium oxide used in the impregnation procedure may be amorphous, partly crystalline or crystalline.

The catalyst compositions of the present invention may also be prepared by mixing multiple compositions, each comprising chromium, oxygen, and at least one modifier metal (with and without fluorinating treatment) provided that the final composition comprises at least two of the modifier metals. By mixing is meant physically mixing two or more different compositions. Typically, each composition mixed is in the form of a powder or granulated material (e.g., pellets). Suitable means of mixing powders and granulated solids are well known in the art. Examples include compositions prepared by mixing a composition comprising chromium, oxygen and gold with a composition comprising chromium, oxygen and silver; compositions prepared by mixing a composition comprising chromium, oxygen and gold with a composition comprising chromium, oxygen and palladium; compositions prepared by mixing a composition comprising chromium, oxygen and palladium with a composition comprising chromium, oxygen and silver; compositions prepared by mixing a composition comprising chromium, oxygen and gold with a composition comprising chromium, oxygen and silver and a composition comprising chromium, oxygen and palladium; compositions prepared by mixing a composition comprising chromium, oxygen, gold and silver with a composition comprising chromium, oxygen, gold and palladium; and compositions prepared by mixing a composition comprising chromium, oxygen, and gold at one concentration and silver with a composition comprising chromium, oxygen, gold at a different concentration and silver. Such compositions can be prepared, for example, by first preparing the selected individual compositions (e.g., by impregnation or co-precipitation) and mixing them in a suitable mixing apparatus prior to use as catalysts. Mixing can be accomplished before or after a calcination step. The mixture of multiple compositions may be treated with a fluorinating agent. If a fluorinating treatment is desired, it may be carried out prior to mixing or after mixing. Of note is a catalyst composition that comprises a mixture of a composition comprising chromium, oxygen, gold and silver with a composition comprising chromium, oxygen, gold and palladium. Also of note is a catalyst composition that comprises a mixture of a composition comprising chromium, oxygen, gold and silver with a composition comprising chromium, oxygen, silver and palladium. Also of note is a catalyst composition that comprises a mixture of a composition comprising chromium, oxygen, gold and palladium with a composition comprising chromium, oxygen, silver and palladium.

The modifier metal-containing chromium oxide catalysts of the present invention can be formed into various shapes such as pellets, granules, and extrudates for use in packing reactors. They can also be used in powder forms.

The catalyst compositions of this invention may further comprise one or more additives in the form of metal compounds. Such additives may alter the selectivity and/or the activity of the modifier metal-containing chromium oxide catalyst compositions or the fluorinated modifier metal-containing chromium oxide catalyst compositions. Suitable additives can be selected from the group consisting of the fluorides, oxides, and oxyfluoride compounds of Mg, Ca, Sc, Y, La, Ti, Zr, W, Mn, Re, Fe, Ru, Co, Rh, Ir, Ni, Pt, Ce, and Zn.

The total content of the additive(s) in the catalyst compositions of the present invention may be from about 0.05 weight % to about 10 weight % based on the total metal content of the catalyst compositions. The additives may be incorporated into the catalyst compositions of the present invention by standard procedures such as by impregnation or during co-precipitation of the modifier metal and chromium salts.

The catalyst compositions of the present invention can be treated with a fluorinating agent to form catalyst compositions comprising chromium, oxygen, modifier metals and fluorine as essential elements. Typically, prior to being used as catalysts, (e.g. for changing the fluorine distribution of hydrocarbons and/or halogenated hydrocarbon compounds) the calcined catalyst compositions of the present invention will be pre-treated with a fluorinating agent. Typically this fluorinating agent is HF though other materials may be used such as sulfur tetrafluoride, carbonyl fluoride, and fluorinated hydrocarbon compounds such as trichlorofluoromethane, dichlorodifluoromethane, chlorodifluoromethane, trifluoromethane, and 1,1,2-trichlorotrifluoroethane. This pretreatment can be accomplished, for example, by placing the catalyst composition in a suitable container which can also be the reactor to be used to perform the process in the present invention, and thereafter, passing HF over the calcined catalyst composition so as to partially saturate the catalyst composition with HF. This can be conveniently carried out by passing HF over the catalyst composition for a period of time, for example, about 0.1 to about 10 hours at a temperature of, for example, about 200° C. to about 450° C. Nevertheless, this pre-treatment is not essential.

The catalyst compositions of the present invention (with and without fluorinating treatment) can be used for changing the fluorine distribution in a hydrocarbon and/or a halogenated hydrocarbon. The fluorine distribution in a hydrocarbon or a halogenated hydrocarbon can be changed by increasing the fluorine content of the hydrocarbon or the halogenated hydrocarbon. The fluorine distribution of a halogenated hydrocarbon can also be changed by decreasing the fluorine content of the halogenated hydrocarbon and/or rearranging the placement of fluorine atoms on the carbon atoms of the halogenated hydrocarbon. Of note are processes where the fluorine distribution in halogenated hydrocarbons containing from one to twelve carbon atoms is changed, particularly processes where the fluorine distribution in halogenated hydrocarbons containing from one to six carbon atoms is changed. Also of note are processes where the fluorine content of hydrocarbons containing from one to twelve carbon atoms is increased, particularly processes where the fluorine content in hydrocarbons containing one to six carbon atoms is increased. Processes for changing the fluorine distribution in halogenated hydrocarbons include fluorination, chlorofluorination, isomerization, disproportionation, dehydrofluorination and chlorodefluorination. The process is characterized by using as the catalyst a composition comprising chromium, oxygen, and modifier metals as essential constituent elements (e.g., a composition comprising chromium, oxygen, gold, silver, and fluorine as essential constituent elements, a composition comprising chromium, oxygen, gold, palladium, and fluorine as essential constituent elements, or a composition comprising chromium, oxygen, palladium, silver, and fluorine as essential constituent elements). Suitable catalyst compositions include those comprising chromium oxide and modifier metals and/or those prepared by treating compositions comprising chromium oxide and modifier metals with a fluorinating agent.

Saturated halogenated hydrocarbons suitable for fluorination, chlorofluorination, isomerization, disproportionation, dehydrofluorination and chlorodefluorination processes of this invention are typically those which have the formula CₙHₐBr_{b}Cl_{c}F_{d}, wherein n is an integer from 1 to 6, a is an integer from 0 to 12, b is an integer from 0 to 4, c is an integer from 0 to 13, d is an integer from 0 to 13, the sum of b, c and d is at least 1 and the sum of a, b, c, and d is equal to 2n+2, provided that n is at least 2 for isomerization, and dehydrofluorination processes and n is at least 1 for the disproportionation process, a is at least 1 for dehydrofluorination processes, b is 0 for chlorodefluorination processes, b+c is at least 1 for fluorination processes and is 0 for dehydrofluorination processes, a+b+c is at least 1 for fluorination, chlorofluorination, isomerization, disproportionation and dehydrofluorination processes and d is at least 1 for isomerization, disproportionation, dehydrofluorination and chlorodefluorination processes. Typical unsaturated halogenated hydrocarbons suitable for fluorination, chlorofluorination, isomerization, disproportionation, and chlorodefluorination processes of this invention are those which have the formula CₚHₑBr_{f}Cl_{g}Fₕ, wherein p is an integer from 2 to 6, e is an integer from 0 to 10, f is an integer from 0 to 2, g is an integer from 0 to 12, h is an integer from 0 to 11, the sum of f, g and h is at least 1 and the sum of e, f, g, and h is equal to 2p, provided that f is 0 for chlorodefluorination processes, e+f+g is at least 1 for isomerization and disproportionation processes and h is at least 1 for isomerization, disproportionation and chlorodefluorination processes. Typical of saturated hydrocarbons suitable for chlorofluorination are those which have the formula C_{q}Hᵣ where q is an integer from 1 to 6 and r is 2q+2. Typical of unsaturated hydrocarbons suitable for fluorination and chlorofluorination are those which have the formula CᵢHⱼ where i is an integer from 2 to 6 and j is 2i.

### Fluorination

Included in this invention is a process for increasing the fluorine content of a halogenated hydrocarbon compound or an unsaturated hydrocarbon compound by reacting said compound with hydrogen fluoride in the vapor phase in the presence of a catalyst of the present invention. The process is characterized by using as the catalyst a composition comprising chromium, oxygen, and modifier metals as essential constituent elements (e.g., a composition comprising chromium, oxygen, modifier metals, and fluorine as essential constituent elements). Suitable catalyst compositions include those comprising chromium oxide and modifier metals and/or those prepared by treating compositions comprising chromium oxide and modifier metals with a fluorinating agent. The catalyst composition may optionally contain additional components such as additives to alter the activity and/or the selectivity of the catalyst.

Halogenated hydrocarbon compounds suitable as starting materials for the fluorination process of this invention may be saturated or unsaturated. Saturated halogenated hydrocarbon compounds suitable for the fluorination processes of this invention include those of the general formula CₙHₐBr_{b}Cl_{c}F_{d}, wherein n is an integer from 1 to 6, a is an integer from 0 to 12, b is an integer from 0 to 4, c is an integer from 0 to 13, d is an integer from 0 to 13, and the sum of a, b, c, and d is equal to 2n+2, provided that b+c is at least 1. Unsaturated halogenated hydrocarbon compounds suitable for the fluorination processes of this invention include those of the general formula CₚHₑBr_{f}Cl_{g}Fₕ, wherein p is an integer from 2 to 6, e is an integer from 0 to 10, f is an integer from 0 to 2, g is an integer from 0 to 12, h is an integer from 0 to 11, the sum of f, g and h is at least 1 and the sum of e, f, g, and h is equal to 2p. Unsaturated hydrocarbons suitable for fluorination are those which have the formula CiHj where i is an integer from 2 to 6 and j is 2i. The fluorine content of saturated compounds of the formula CₙHₐBr_{b}Cl_{c}F_{d}, unsaturated compounds of the formula CₚHₑBr_{f}Cl_{g}Fₕ and/or unsaturated compounds of the formula CᵢHⱼ may be increased by reacting said compounds with HF in the vapor phase in the presence of the catalyst composition of the present invention described herein. Such a process is referred to herein as a vapor phase fluorination reaction.

Further information on the fluorination of CFC-1213xa and further reaction of products obtained from the fluorination reaction is provided in U.S. Patent Applications 60/927,843 and 60/927,842 [FL-1362 US PRV and FL-1363 US PRV] filed May 4, 2007 and hereby incorporated by reference herein in their entirety.

The vapor phase fluorination reactions are typically conducted at temperatures of from about 150° C. to 500° C. For saturated compounds the fluorination is preferably carried out from about 175° C. to 400° C. and more preferably from about 200° C. to about 350° C. For unsaturated compounds the fluorination is preferably carried out from about 150° C. to 350° C. and more preferably from about 175° C. to about 300° C.

The vapor phase fluorination reactions are typically conducted at atmospheric and superatmospheric pressures. For reasons of convenience in downstream separation processes (e.g., distillation), pressures of up to about 30 atmospheres may be employed.

The vapor phase fluorination reactions are typically conducted in a tubular reactor. The reactor and its associated feed lines, effluent lines, and associated units should be constructed of materials resistant to hydrogen fluoride and hydrogen chloride. Typical materials of construction, well-known to the fluorination art, include stainless steels, in particular of the austenitic type, the well-known high nickel alloys, such as Monel® nickel-gold alloys, Hastelloy® nickel-based alloys and, Inconel® nickel-chromium alloys, and gold-clad steel.

The contact time in the reactor is typically from about 1 to about 120 seconds. Of note are contact times of from about 5 to about 60 seconds.

The amount of HF reacted with the unsaturated hydrocarbons or halogenated hydrocarbon compounds should be at least a stoichiometric amount. The stoichiometric amount is based on the number of Br and/or Cl substituents to be replaced by F in addition to one mole of HF to saturate the carbon-carbon double bond if present. Typically, the molar ratio of HF to the said compounds of the formulas CₙHₐBr_{b}Cl_{c}F_{d}, CₚHₑBr_{f}Cl_{g}Fₕ, and CᵢHⱼ can range from about 0.5:1 to about 100:1, preferably from about 2:1 to about 50:1, and more preferably from about 3:1 to about 20:1. In general, with a given catalyst composition, the higher the temperature and the longer the contact time, the greater is the conversion to fluorinated products. The above variables can be balanced, one against the other, so that the formation of higher fluorine substituted products is maximized.

Examples of saturated compounds of the formula CₙHₐBr_{b}Cl_{c}F_{d} which may be reacted with HF in the presence of the catalyst of this invention include CH₂Cl₂, CH₂Br₂, CHCl₃, CCl₄, CBr₄, C₂Cl₆, C₂BrCl₅, C₂Cl₅F, C₂Cl₄F₂, C₂Cl₃F₃, C₂Cl₂F₄, C₂ClF₅, C₂HCl₅, C₂HCl₄F, C₂HCl₃F₂, C₂HCl₂F₃, C₂HClF₄, C₂HBrF₄, C₂H₂Cl₄, C₂H₂Cl₃F, C₂H₂Cl₂F₂, C₂H₂ClF₃, C₂H₃Cl₃, C₂H₃Cl₂F, C₂H₃ClF₂, C₂H₄Cl₂, C₂H₄ClF, C₃Cl₆F₂, C₃Cl₅F₃, C₃Cl₄F₄, C₃Cl₃F₅, C₃HCl₇, C₃HCl₆F, C₃HCl₅F₂, C₃HCl₄F₃, C₃HCl₃F₄, C₃HCl₂F₅, C₃H₂Cl₆, C₃H₂BrCl₅, C₃H₂Cl₅F, C₃H₂Cl₄F₂, C₃H₂Cl₃F₃, C₃H₂Cl₂F₄, C₃H₂ClF₅, C₃H₃Cl₅, C₃H₃Cl₄F, C₃H₃Cl₃F₂, C₃H₃Cl₂F₃, C₃H₃ClF₄, C₃H₄Cl₄, C₄H₆Cl₄, C₄H₄Cl₆, C₄H₅Cl₅, C₄H₅Cl₄F, C₄H₄Cl₃F₃, C₄H₄Cl₄F₂, C₄H₄Cl₅F, C₅H₂Cl₄F₆, C₅H₂Cl₅F₅, C₅H₃Cl₄F₅, C₅H₃Cl₅F₄, and C₅H₄Cl₈.

Specific examples of vapor phase fluorination reactions of saturated halogenated hydrocarbon compounds which may be carried out under the conditions described above using the catalysts of this invention include the conversion of CH₂Cl₂ to CH₂F₂, the conversion of CHCl₃ to a mixture of CHCl₂F, CHClF₂, and CHF₃, the conversion of CH₃CHCl₂ to a mixture of CH₃CHClF and CH₃CHF₂, the conversion of CH₂ClCH₂Cl to a mixture of CH₃CHClF and CH₃CHF₂, the conversion of CH₃CCl₃ to a mixture of CH₃CCl₂F, CH₃CClF₂, and CH₃CF₃, the conversion of CH₂ClCF₃ to CH₂FCF₃, the conversion of CHCl₂CF₃ to a mixture of CHClFCF₃ and CHF₂CF₃, the conversion of CHClFCF₃ to CHF₂CF₃, the conversion of CHBrFCF₃ to CHF₂CF₃, the conversion of CCl₃CF₂CCl₃ to a mixture of CCl₂FCF₂CClF₂ and CClF₂CF₂CClF₂, the conversion of CCl₃CH₂CCl₃ to CF₃CH₂CClF₂ and CF₃CH₂CF₃, the conversion of CCl₃CH₂CHCl₂ to a mixture of CF₃CH₂CHF₂, CF₃CH═CHCl, and CF₃CH═CHF, the conversion of CF₃CCl₂CClF₂ to a mixture of CF₃CCl₂CF₃, and CF₃CClFCF₃, the conversion of CF₃CCl₂CF₃ to CF₃ClFCF₃, and the conversion of a mixture comprising CF₃CF₂CHCl₂ and CClF₂CF₂CHClF to a mixture of CF₃CF₂CHClF and CF₃CF₂CHF₂.

Examples of unsaturated compounds of the formula CₚHₑBr_{f}Cl_{g}Fₕ and CᵢHⱼ which may be reacted with HF in the presence of the catalysts of this invention include C₂Cl₄, C₂BrCl₃, C₂Cl₃F, C₂Cl₂F₂, C₂ClF₃, C₂F₄, C₂HCl₃, C₂HBrCl₂, C₂HCl₂F, C₂HClF₂, C₂HF₃, C₂H₂Cl₂, C₂H₂ClF, C₂H₂F₂, C₂H₃C₁, C₂H₃F, C₂H₄, C₃H₆, C₃H₅C₁, C₃H₄Cl₂, C₃H₃Cl₃, C₃H₂Cl₄, C₃HCl₅, C₃Cl₆, C₃Cl₅F, C₃Cl₄F₂, C₃Cl₃F₃, C₃Cl₂F₄, C₃ClF₅, C₃HF₅, C₃H₂F₄, C₃F₆, C₄Cl₈, C₄Cl₂F₆, C₄ClF₇, C₄H₂F₆, C₄H₂ClF₅, C₄H₂Cl₂F₄, C₄H₂Cl₃F₃, C₄HClF₆ and C₅H₂Cl₄F₅.

Specific examples of vapor phase fluorination reactions of unsaturated halogenated hydrocarbon compounds which may be carried out using the catalysts of this invention include the conversion of CHCl═CCl₂ to a mixture of CH₂ClCF₃ and CH₂FCF₃, the conversion of CCl₂═CCl₂ to a mixture of CHCl₂CF₃, CHClFCF₃, and CHF₂CF₃, the conversion of CCl₂═CH₂ to a mixture of CH₃CCl₂F, CH₃CClF₂, and CH₃CF₃, the conversion of CH₂═CHCl to a mixture of CH₃CHClF and CH₃CHF₂, the conversion of CF₂═CH₂ to CH₃CF₃, the conversion of CCl₂═CClCF₃ to a mixture of CF₃CHClCClF₂, CF₃CHClCF₃, and/or CF₃CCl═CF₂, the conversion of CF₃CF═CF₂ to CF₃CHFCF₃, the conversion of CF₃CH═CF₂ to CF₃CH₂CF₃, and the conversion of CF₃CH═CHF to CF₃CH₂CHF₂.

Of note is a catalytic process for producing a mixture of 2-chloro-1,1,1,3,3,3-hexafluoropropane (i.e., CF₃CHClCF₃ or HCFC-226da) and 2-chloro-pentafluoropropene (i.e., CF₃CCl═CF₂ or CFC-1215xc) by the vapor phase fluorination reactions of a hexahalopropene of the formula C₃Cl₆₋ₓFₓ, wherein x equals 0 to 4. Preferred hexahalopropenes of the formula C₃Cl₆₋ₓFₓ include 1,1,2-trichloro-3,3,3-trifluoro-1-propene (i.e., CCl₂═CClCF₃ or CFC-1213xa) and hexachloropropene (i.e., CCl₂═CClCCl₃). The mixture of HCFC-226da and CFC-1215xc is produced by reacting the above unsaturated compounds with HF in the vapor phase in the presence of the catalysts of this invention at temperatures from about 150° C. to about 400° C., preferably from about 200° C. to about 350° C. The amount of HF fed to the reactor should be at least a stoichiometric amount as define above. In the case of fluorination of CFC-1213xa to a mixture of HCFC-226da and CFC-1215xc, the stoichiometric ratio of HF to CFC-1213xa is 3:1. Preferred ratios of HF to C₃Cl₆₋ₓFₓ starting material(s) are typically in the range of from about the stoichiometric ratio to about 25:1. Preferred contact times are typically in the range of from 1 to 60 seconds.

Mixtures of saturated halogenated hydrocarbon compounds or mixtures of unsaturated hydrocarbons and/or halogenated hydrocarbon compounds may also be used in the vapor phase fluorination reactions as well as mixtures comprising both unsaturated hydrocarbons and halogenated hydrocarbon compounds. Specific examples of mixtures of saturated halogenated hydrocarbon compounds and mixtures of unsaturated hydrocarbons and unsaturated halogenated hydrocarbon compounds that may be subjected to vapor phase fluorination using the catalysts of this invention include a mixture of CH₂Cl₂ and CCl₂═CCl₂, a mixture of CCl₂FCClF₂ and CCl₃CF₃, a mixture of CCl₂═CCl₂ and CCl₂═CClCCl₃, a mixture of CH₂═CHCH₃ and CH₂═CClCH₃, a mixture of CH₂Cl₂ and CH₃CCl₃, a mixture of CHF₂CClF₂ and CHClFCF₃, a mixture of CHCl₂CCl₂CH₂Cl and CCl₃CHClCH₂Cl, a mixture of CHCl₂CH₂CCl₃ and CCl₃CHClCH₂Cl, a mixture of CHCl₂CHClCCl₃, CCl₃CH₂CCl₃, and CCl₃CCl₂CH₂Cl, a mixture of CHCl₂CH₂CCl₃ and CCl₃CH₂CCl₃, a mixture of and CF₃CH₂CCl₂F and CF₃CH═CCl₂, and a mixture of CF₃CH═CHCl and CF₃CH═CCl₂.

### Chlorofluorination

Included in this invention is a process for increasing the fluorine content of a halogenated hydrocarbon compound or a hydrocarbon compound by reacting said compound with hydrogen fluoride (HF) and chlorine (Cl₂) in the vapor phase in the presence of a catalyst. The process is characterized by using as the catalyst a composition comprising chromium, oxygen, and modifier metals as essential constituent elements (e.g., a composition comprising chromium, oxygen, modifier metals, and fluorine as essential constituent elements). Suitable catalyst compositions include those comprising chromium oxide and modifier metals and/or those prepared by treating compositions comprising chromium oxide and modifier metals with a fluorinating agent. The catalyst composition may optionally contain additional components such as additives to alter the activity and/or the selectivity of the catalyst.

Halogenated hydrocarbon compounds suitable as starting materials for the chlorofluorination process of this invention may be saturated or unsaturated. Saturated halogenated hydrocarbon compounds suitable for the chlorofluorination processes of this invention include those of the general formula CₙHₐBr_{b}Cl_{c}F_{d}, wherein n is an integer from 1 to 6, a is an integer from 0 to 12, b is an integer from 0 to 4, c is an integer from 0 to 13, d is an integer from 0 to 13, the sum of b, c and d is at least 1 and the sum of a, b, c, and d is equal to 2n+2, provided that a+b+c is at least 1. Preferred chlorofluorination processes include those involving said saturated starting materials where a is at least 1. Saturated hydrocarbon compounds suitable for chlorofluorination are those which have the formula C_{q}Hᵣ where q is an integer from 1 to 6 and r is 2q+2. Unsaturated halogenated hydrocarbon compounds suitable for the chlorofluorination processes of this invention include those of the general formula CₚHₑBr_{f}Cl_{g}Fₕ, wherein p is an integer from 2 to 6, e is an integer from 0 to 10, f is an integer from 0 to 2, g is an integer from 0 to 12, h is an integer from 0 to 11, the sum of f, g and h is at least 1 and the sum of e, f, g, and h is equal to 2p. Unsaturated hydrocarbon compounds suitable for fluorination are those which have the formula CᵢHⱼ where i is an integer from 2 to 6 and j is 2i. The fluorine content of saturated compounds of the formula CₙHₐBr_{b}Cl_{c}F_{d} and C_{q}Hᵣ and/or unsaturated compounds of the formula CₚHₑBr_{f}Cl_{g}Fₕ and CᵢHⱼ may be increased by reacting said compounds with HF and Cl₂ in the vapor phase in the presence of a catalyst mentioned herein. Such a process is referred to herein as a vapor phase chlorofluorination reaction.

The conditions of the vapor phase chlorofluorination reactions are similar to those described above for vapor phase fluorination reactions in terms of the temperature ranges, contact times, pressures, and mole ratios of HF to the halogenated hydrocarbon compounds. The amount of chlorine (Cl₂) fed to the reactor is based on whether the halogenated hydrocarbon compounds fed to the reactor is unsaturated and the number of hydrogens in CₙHₐBr_{b}Cl_{c}F_{d}, C_{q}Hᵣ, CₚHₑBr_{f}Cl_{g}Fₕ, and CᵢHⱼ that are to be replaced by chlorine and fluorine. One mole of Cl₂ is required to saturate a carbon-carbon double bond and a mole of Cl₂ is required for each hydrogen to be replaced by chlorine or fluorine. A slight excess of chlorine over the stoichiometric amount may be necessary for practical reasons, but large excesses of chlorine will result in complete chlorofluorination of the products. The ratio of Cl₂ to halogenated hydrocarbon compound is typically from about 1:1 to about 10:1.

Specific examples of vapor phase chlorofluorination reactions of saturated halogenated hydrocarbon compounds of the general formula CₙHₐBr_{b}Cl_{c}F_{d} and saturated hydrocarbon compounds of the general formula C_{q}Hᵣ which may be carried out using the catalysts of this invention include the conversion of C₂H₆ to a mixture containing CH₂ClCF₃, the conversion of CH₂ClCF₃ to a mixture of CHClFCF₃ and CHF₂CF₃, the conversion of CCl₃CH₂CH₂Cl to a mixture of CF₃CCl₂CClF₂, CF₃CCl₂CF₃, CF₃CClFCClF₂, and CF₃CClFCF₃, the conversion of CCl₃CH₂CHCl₂ to a mixture of CF₃CCl₂CClF₂, CF₃CCl₂CF₃, CF₃CClFCClF₂, and CF₃CClFCF₃, the conversion of CCl₃CHClCH₂Cl to a mixture of CF₃CCl₂CClF₂, CF₃CCl₂CF₃, CF₃CClFCClF₂, and CF₃CClFCF₃, the conversion of CHCl₂CCl₂CH₂Cl to a mixture of CF₃CCl₂CClF₂, CF₃CCl₂CF₃, CF₃CClFCClF₂, and CF₃CClFCF₃, the conversion of CCl₃CH₂CH₂Cl to a mixture of CF₃CCl₂CHF₂, CF₃CClFCHF₂, CF₃CClFCClF₂, and CF₃CCl₂CF₃, and the conversion of CCl₃CH₂CHCl₂ to a mixture of CF₃CCl₂CHF₂, CF₃CClFCHF₂, CF₃CClFCClF₂, and CF₃CCl₂CF₃.

Specific examples of vapor phase chlorofluorination reactions of unsaturated halogenated hydrocarbon compounds of the general formula CₚHₑBr_{f}Cl_{g}Fₕ and unsaturated hydrocarbon compounds of the general formula CᵢHⱼ which may be carried out using the catalysts of this invention include the conversion of C₂H₄ to a mixture of CCl₃CClF₂, CCl₂FCCl₂F, CClF₂CCl₂F, CCl₃CF₃, CF₃CCl₂F, and CClF₂CClF₂, the conversion of C₂Cl₄ to a mixture of CCl₃CClF₂, CCl₂FCCl₂F, CClF₂CCl₂F, CCl₃CF₃, CF₃CCl₂F, and CClF₂CClF₂, and the conversion of C₃H₆ or CF₃CCl═CCl₂ to a mixture of CF₃CCl₂CClF₂, CF₃CCl₂CF₃, CF₃CClFCClF₂, and CF₃CClFCF₃.

Of note is a catalytic process for producing a mixture of 1,2,2-trichloro-1,1,3,3,3-pentafluoropropane (i.e., CClF₂CCl₂CF₃ or CFC-215aa), 1,1,2-trichloro-1,2,3,3,3-pentafluoropropane (i.e., CCl₂FCClFCF₃ or CFC-215bb), 2,2-dichloro-1,1,1,3,3,3-hexafluoropropane (i.e., CF₃CCl₂CF₃ or CFC-216aa), 1,2-dichloro-1,1,1,3,3,3-hexafluoropropane (i.e., CClF₂CClFCF₃ or CFC-216ba), and 2-chloro-1,1,1,2,3,3,3-heptafluoropropane (i.e., CF₃CClFCF₃ or CFC-217ba), by the chlorofluorination of a hexahalopropene of the formula C₃Cl₆₋ₓFₓ, wherein x equals 0 to 4. Preferred hexahalopropenes of the formula C₃Cl₆₋ₓFₓ include 1,1,2-trichloro-3,3,3-trifluoro-1-propene (i.e., CCl₂═CClCF₃ or CFC-1213xa) and hexachloropropene (i.e., CCl₂═CClCCl₃). The mixture of CFC-215aa, -215bb, -216aa, -216ba, and -217ba is produced by reacting the above unsaturated compounds with Cl₂ and HF in the vapor phase in the presence of the catalysts of this invention at temperatures from about 150° C. to about 450° C., preferably about 250° C. to 400° C.

The amount of HF fed to the reactor should be at least a stoichiometric amount as defined above. In the case of chlorofluorination of CFC-1213xa to a mixture of chlorofluoropropanes having an average number of fluorine substituents of six, the stoichiometric ratio of HF to CFC-1213xa is 3:1. Preferred ratios of HF to C₃Cl₆₋ₓFₓ starting material(s) are typically in the range of from about the stoichiometric ratio to about 30:1, more preferably from about 8:1 to about 25:1.

The amount of chlorine fed to the reactor should be at least one mole of chlorine per mole of hexahalopropene fed to the reactor. Preferred molar ratios of Cl₂ to CFC-1213xa are from about 1:1 to about 5:1. Of note are contact times of from about 5 seconds to about 60 seconds.

Further information on the chlorofluorination of CFC-1213xa and further reaction of products obtained from the chlorofluorination reaction is provided in U.S. Patent Applications 60/927,839, 60/927,848, 60/927,838 and 60/927,847 [FL-1364 US PRV, FL-1365 US PRV, FL-1366 US PRV, and FL-1367 US PRV] filed May 4, 2007, all hereby incorporated by reference herein in their entirety.

Mixtures of saturated hydrocarbon compounds and saturated halogenated hydrocarbon compounds and mixtures of unsaturated hydrocarbon compounds and unsaturated halogenated hydrocarbon compounds as well as mixtures comprising both saturated and unsaturated compounds may be chlorofluorinated using the catalysts of the present invention. Specific examples of mixtures of saturated and unsaturated hydrocarbons and halogenated hydrocarbons that may be used include a mixture of CCl₂═CCl₂ and CCl₂═CClCCl₃, a mixture of CHCl₂CCl₂CH₂Cl and CCl₃CHClCH₂Cl, a mixture of CHCl₂CH₂CCl₃ and CCl₃CHClCH₂Cl, a mixture of CHCl₂CHClCCl₃, CCl₃CH₂CCl₃, and CCl₃CCl₂CH₂Cl, a mixture of CHF₂CH₂CF₃ and CHCl═CHCF₃, and a mixture of CH₂═CH₂ and CH₂═CHCH₃.

### Isomerization and Disproportionation

Included in this invention is a process for changing the fluorine distribution in a halogenated hydrocarbon compound by isomerizing said halogenated hydrocarbon compound in the presence of a catalyst. The process is characterized by using as the catalyst a composition comprising chromium, oxygen, and modifier metals as essential constituent elements (e.g., a composition comprising chromium, oxygen, modifier metals, and fluorine as essential constituent elements). Suitable catalyst compositions include those comprising chromium oxide and modifier metals and/or those prepared by treating compositions comprising chromium oxide and modifier metals with a fluorinating agent. The catalyst composition may optionally contain additional components such as additives to alter the activity and/or the selectivity of the catalyst.

Also included in this invention is a process for changing the fluorine distribution in a halogenated hydrocarbon compound by disproportionating said halogenated hydrocarbon compound in the vapor phase in the presence of a catalyst. The process is characterized by using as the catalyst a composition comprising chromium oxide and modifier metals and/or a chromium-containing catalyst composition prepared by treating said composition comprising chromium oxide and modifier metals with a fluorinating agent. The catalyst composition may optionally contain additional components such as additives to alter the activity and/or the selectivity of the catalyst.

Halogenated hydrocarbon compounds suitable as starting materials for the isomerization and disproportionation processes of this invention may be saturated or unsaturated. Saturated halogenated hydrocarbon compounds suitable for the isomerization and disproportionation processes of this invention include those of the general formula CₙHₐBr_{b}Cl_{c}F_{d}, wherein n is an integer from 2 to 6, a is an integer from 0 to 12, b is an integer from 0 to 4, c is an integer from 0 to 13, d is an integer from 1 to 13, and the sum of a, b, c, and d is equal to 2n+2, provided that a+b+c is at least 1. Unsaturated halogenated hydrocarbon compounds suitable for the isomerization and disproportionation processes of this invention include those of the general formula CₚHₑBr_{f}Cl_{g}Fₕ, wherein p is an integer from 2 to 6, e is an integer from 0 to 10, f is an integer from 0 to 2, g is an integer from 0 to 12, h is an integer from 1 to 11, and the sum of e, f, g, and h is equal to 2p, provided that the sum of e+f+g is at least 1.

In one embodiment of the present invention, the fluorine distribution of a halogenated hydrocarbon compound is changed by rearranging the H, Br, Cl, and F substituents in the molecule (typically to a thermodynamically preferred arrangement) while maintaining the same number of the H, Br, Cl, and F substituents, respectively. This process is referred to herein as isomerization.

In another embodiment of the present invention, the fluorine distribution of a halogenated hydrocarbon compound is changed by exchanging at least one F substituent of the halogenated hydrocarbon starting material with at least one H, Br and/or Cl substituent of another molecule of the halogenated hydrocarbon starting material so as to result in the formation of one or more halogenated hydrocarbon compounds having a decreased fluorine content compared to the halogenated hydrocarbon starting material and one or more halogenated hydrocarbon compounds having an increased fluorine content compared to the halogenated hydrocarbon starting material. This process is referred to herein as disproportionation.

In another embodiment of the present invention, both isomerization and disproportionation reactions may occur simultaneously.

The isomerization and disproportionation (see disproportionation paragraph below) reactions are typically conducted at temperatures of from about 150° C. to 500° C., preferably from about 200° C. to about 400° C. The contact time in the reactor is typically from about 1 to about 120 seconds and preferably from about 5 to about 60 seconds. The isomerization and disproportionation reactions may be carried out in the presence of an inert gas such as helium, argon, or nitrogen though this is not preferred. The isomerization and disproportionation reactions may be carried out in the presence of HF and HCl, but this is not preferred.

Specific examples of vapor phase isomerization reactions which may be carried out using the catalysts of this invention include the conversion of CClF₂CCl₂F to CCl₃CF₃, the conversion of CClF₂CClF₂ to CF₃CCl₂F, the conversion of CHF₂CClF₂ to CF₃CHClF, the conversion of CHF₂CHF₂ to CF₃CH₂F, the conversion of CF₃CClFCClF₂ to CF₃CCl₂CF₃, and the conversion of CF₃CHFCHF₂ to CF₃CH₂CF₃.

Specific examples of vapor phase disproportionation reactions which may be carried out using the catalysts of this invention include the conversion of CClF₂CClF₂ to a mixture of CClF₂CCl₂F, CCl₃CF₃, and CF₃CClF₂, and the conversion of CHClFCF₃ to a mixture of CHCl₂CF₃, and CHF₂CF₃.

Of note is a process for the conversion of a mixture of 2-chloro-1,1,2,2-tetrafluoroethane (i.e., CHF₂CClF₂ or HCFC-124a) and 2-chloro-1,1,1,2-tetrafluoroethane (i.e., CF₃CHClF or HCFC-124) to a mixture comprising 2,2-dichloro-1,1,1-trifluoroethane (i.e., CHCl₂CF₃ or HCFC-123) and 1,1,1,2,2-pentafluoroethane (i.e., CF₃CHF₂ or HFC-125) in addition to unconverted starting materials. The mixture comprising HFC-125 and HCFC-123 may be obtained in the vapor phase by contacting a mixture of HCFC-124a and -124 over the catalysts of this invention optionally in the presence of a diluent selected from the group consisting of HF, HCl, nitrogen, helium, argon, and carbon dioxide. If used, the diluent gas, may be present in a molar ratio of diluent to haloethane of from about 1:1 to about 5:1.

### Dehydrofluorination

Included in this invention is a process for decreasing the fluorine content of a halogenated hydrocarbon compound by dehydrofluorinating said halogenated hydrocarbon compound in the presence of a catalyst. The process is characterized by using as the catalyst a composition comprising chromium, oxygen, and modifier metals as essential constituent elements (e.g., a composition comprising chromium, oxygen, modifier metals, and fluorine as essential constituent elements). Suitable catalyst compositions include those comprising chromium oxide and modifier metals and/or those prepared by treating compositions comprising chromium oxide and modifier metals with a fluorinating agent. The catalyst composition may optionally contain additional components such as additives to alter the activity and/or the selectivity of the catalyst.

Halogenated hydrocarbon compounds suitable as starting materials for the dehydrofluorination process of this invention are typically saturated. Saturated halogenated hydrocarbon compounds suitable for the dehydrofluorination processes of this invention include those of the general formula CₙHₐF_{d}, wherein n is an integer from 2 to 6, a is an integer from 1 to 12, d is an integer from 1 to 13, and the sum of a and d is equal to 2n+2. The fluorine content of saturated compounds of the formula CₙHₐF_{d} may be decreased in the presence of catalysts of the present invention. This decrease in fluorine content is typically associated with removal of hydrogen fluoride (HF) from the molecule and is referred to herein as dehydrofluorination.

The dehydrofluorination reactions are typically conducted at temperatures of from about 200° C. to about 500° C., preferably from about 300° C. to about 450° C. The contact time in the reactor is typically from about 1 to about 360 seconds. Of note are contact times of from about 5 to about 120 seconds. Carrying out the dehydrofluorination reactions in the presence of an inert gas such as helium, argon, or nitrogen promotes the dissociation of the fluorinated carbon compound, but this practice can also lead to difficulties in separation and is not preferred. The product of dehydrofluorination reaction consists of HF and the unsaturated fluorinated carbon compound resulting from loss of HF from the starting material.

Specific examples of vapor phase dehydrofluorination reactions which may be carried out using the catalysts of this invention include the conversion of CH₃CHF₂ to CH₂═CHF, the conversion of CH₃CF₃ to CH₂═CF₂, the conversion of CF₃CH₂F to CF₂═CHF, the conversion of CHF₂CH₂CF₃ to CHF═CHCF₃, the conversion of CHF₂CHFCF₃ to CHF═CFCF₃, the conversion of CH₃CF₂CF₃ to CH₂═CFCF₃, the conversion of CH₂FCF₂CF₃ to CHF═CFCF₃, and the conversion of CF₃CH₂CF₃ to CF₃CH═CF₂.

Of note is a catalytic process for producing fluoroethene (i.e., CH₂═CHF or vinyl fluoride) by the dehydrofluorination of a 1,1-difluoroethane (i.e., CHF₂CH₃ or HFC-152a). A mixture comprising vinyl fluoride and unconverted HFC-152a may be obtained in the vapor phase by contacting HFC-152a over the catalysts of this invention optionally in the presence of a diluent selected from the group consisting of HF, nitrogen, helium, argon, and carbon dioxide. The dehydrofluorination is preferably conducted at about 150° C. to about 400° C., more preferably about 250° C. to about 350° C. If used, the diluent gas, may be present in a molar ratio of diluent to haloethane of from about 1:1 to about 5:1. Of note are contact times of from about 10 seconds to about 60 seconds.

### Chlorodefluorination

Included in this invention is a process for decreasing the fluorine content of a halogenated hydrocarbon compound by reacting said halogenated hydrocarbon compound with hydrogen chloride (HCl) in the vapor phase in the presence of a catalyst. The process is characterized by using as the catalyst a composition comprising chromium, oxygen, and modifier metals as essential constituent elements (e.g., a composition comprising chromium, oxygen, modifier metals, and fluorine as essential constituent elements). Suitable catalyst compositions include those comprising chromium oxide and modifier metals and/or those prepared by treating compositions comprising chromium oxide and modifier metals with a fluorinating agent. The catalyst composition may optionally contain additional components such as additives to alter the activity and/or the selectivity of the catalyst.

Halogenated hydrocarbon compounds suitable as starting materials for the chlorodefluorination processes of this invention may be saturated or unsaturated. Saturated halogenated hydrocarbon compounds suitable for the chlorodefluorination processes of this invention include those of the general formula CₙHₐCl_{c}F_{d}, wherein n is an integer from 1 to 6, a is an integer from 0 to 12, c is an integer from 0 to 13, d is an integer from 1 to 13, and the sum of a, c and d is equal to 2n+2. Unsaturated halogenated hydrocarbon compounds suitable for the chlorodefluorination processes of this invention include those of the general formula CₚHₑCl_{g}Fₕ, wherein p is an integer from 2 to 6, e is an integer from 0 to 10, g is an integer from 0 to 12, h is an integer from 1 to 11, and the sum of e, g, and h is equal to 2p. The fluorine content of saturated compounds of the formula CₙHₐCl_{c}F_{d} and/or unsaturated compounds of the formula CₚHₑCl_{g}Fₕ may be decreased by reacting said compounds with HCl in the vapor phase in the presence of catalysts of the present invention. Such a process is referred to herein as a vapor phase chlorodefluorination reaction. Chlorodefluorination is disclosed in U.S. Pat. No. 5,345,017 and U.S. Pat. No. 5,763,698 and the teachings of these two patents are hereby incorporated herein by reference.

The chlorodefluorination reactions are typically conducted at temperatures of from about 250° C. to 450° C., preferably from about 300° C. to about 400° C. The contact time in the reactor is typically from about 1 to about 120 seconds. Of note are contact times of from about 5 to about 60 seconds. The reactions are most conveniently carried out at atmospheric or superatmospheric pressure.

Chlorodefluorinations involving saturated halogenated hydrocarbons are of particular note. The molar ratio of HCl to the saturated halogenated hydrocarbon compound is typically from about 1:1 to about 100:1, preferably from about 3:1 to about 50:1, and most preferably from about 4:1 to about 30:1. In general, with a given catalyst composition, the higher the temperature, the longer the contact time, and the greater the molar ratio of HCl to saturated halogenated hydrocarbon compound, the greater is the conversion to compounds having lower fluorine content. The above variables can be balanced, one against the other, so that the formation of chlorine-substituted products is maximized.

The product of chlorodefluorination reactions typically comprise unreacted HCl, HF, unconverted starting material, and saturated halogenated hydrocarbon compounds having a lower fluorine content than the starting material by virtue of the substitution of one or more fluorine substituents for chlorine.

Specific examples of vapor phase chlorodefluorination reactions which may be carried out using the catalysts of this invention include the conversion of CHF₃ to a mixture of CHCl₃, CHCl₂F, and CHClF₂, the conversion of CClF₂CClF₂ to a mixture of CCl₃CCl₃, CCl₃CCl₂F, CCl₃CClF₂, CCl₂FCCl₂F, CClF₂CCl₂F, and CCl₃CF₃, the conversion of CF₃CClF₂ to a mixture of CCl₃CCl₃, CCl₃CCl₂F, CCl₃CClF₂, CCl₂FCCl₂F, CClF₂CCl₂F, CCl₃CF₃, CClF₂CClF₂, and CF₃CCl₂F, the conversion of CF₃CCl₂CF₃ to a mixture of CF₃CCl₂CClF₂, CF₃CCl₂CCl₂F, CF₃CCl₂CCl₃, and CClF₂CCl₂CCl₃, and the conversion of CF₃CH₂CF₃ to a mixture of CCl₂═CHCF₃, and CCl₂═CClCF₃.

Of note is a catalytic process for producing a mixture containing 1,1-dichloro-3,3,3-trifluoro-1-propene (i.e., CCl₂═CHCF₃ or HCFC-1223za) and 1,1,2-trichloro-3,3,3-trifluoro-1-propene (i.e., CCl₂═CClCF₃ or CFC-1213xa) by the chlorodefluorination of 1,1,1,3,3,3-hexafluoropropane (i.e., CF₃CH₂CF₃ or HFC-236fa) by reaction of HFC-236fa with HCl in the vapor phase in the presence of the catalysts of this invention. The reaction is preferably conducted from about 275° C. to about 450° C., more preferably about 300° C. to about 400° C. with a molar ratio of HCl to HFC-236fa of preferably from about 3:1 to about 20:1. Of note are contacts times of from about 1 second to about 40 seconds. Oxygen in the form of air or co-fed with an inert diluent such as nitrogen, helium, or argon may be added along with the reactants or as a separate catalyst treatment, if desired.

The reaction products obtained by the processes of this invention can be separated by conventional techniques, such as with combinations including, but not limited to, scrubbing, decantation, or distillation. Some of the products of the various embodiments of this invention may form one or more azeotropes with each other or with HF.

The processes of this invention can be carried out readily using well known chemical engineering practices.

### Utility

Some of the hydrofluorocarbon reaction products obtained through use of the catalysts disclosed herein will have desired properties for direct commercial use and/or serve as useful starting materials for the manufacture of hydrofluoroolefins. For example, CH₂F₂ (HFC-32), CHF₂CF₃ (HFC-125), CHF₂CH₃ (HFC-152a), CH₂FCF₃ (HFC-134a), CF₃CH₂CF₃ (HFC-236fa), and CF₃CH₂CHF₂ (HFC-245fa) find application as refrigerants, CH₂FCF₃ (HFC-134a) and CF₃CHFCF₃ (HFC-227ea) find application as propellants, CH₃CHF₂ (HFC-152a) and CF₃CH₂CHF₂ (HFC-245fa) find application as blowing agents, and CHF₂CF₃ (HFC-125), CF₃CH₂CF₃ (HFC-236fa), and CF₃CHFCF₃ (HFC-227ea) find application as fire extinguishants. In addition CF₃CH₂CF₃ can be used to prepare CF₃CH═CF₂, CF₃CH₂CHF₂ can be used to prepare CF₃CH═CHF and CF₃CHFCF₃ can be used to prepare CF₃CF═CF₂.

Some reaction products obtained through the use of this invention are used as chemical intermediates to make useful products. For example, CCl₃CF₃ (CFC-113a) can be used to prepare CFC-114a which can then be converted to CH₂FCF₃ (HFC-134a) by hydrodechlorination. Similarly, CF₃CCl₂CF₃ (CFC-216aa) can be used to prepare CF₃CH₂CF₃ (HFC-236fa) by hydrodechlorination and CF₃CCl═CF₂ (CFC-1215zc) can be used to prepare CF₃CH₂CHF₂ (HFC-245fa) by hydrogenation.

Embodiments of this invention include, but are not limited to:

Embodiment A1. A catalyst composition, comprising chromium, oxygen, and at least two modifier metals selected from the group consisting of gold, silver, and palladium, as essential constituent elements thereof, wherein the total amount of modifier metals is from about 0.05 atom % to about 10 atom % based on the total amount of chromium and modifier metals in the catalyst composition.

Embodiment A2. The catalyst composition of Embodiment A1 further comprising fluorine as an essential constituent element.

Embodiment A3. The catalyst composition of Embodiment A1 comprising gold and silver in a mole ratio of from about 10:1 to about 1:10.

Embodiment A4. The catalyst composition of Embodiment A1 comprising gold and palladium in a mole ratio of from about 10:1 to about 1:10.

Embodiment A5. The catalyst composition of Embodiment A1 comprising silver and palladium in a mole ratio of from about 10:1 to about 1:10.

Embodiment A6. The catalyst composition of Embodiment A1, comprising particles of modifier metals supported on a chromium oxide support.

Embodiment A7. A process for changing the fluorine distribution in a hydrocarbon or halogenated hydrocarbon in the presence of a catalyst, characterized by using the catalyst composition of Embodiment A1 as the catalyst.

Embodiment A8. The process of Embodiment A7 wherein the fluorine content of a halogenated hydrocarbon compound or an unsaturated hydrocarbon compound is increased by reacting said compound with hydrogen fluoride in the vapor phase in the presence of said catalyst composition.

Embodiment A9. The process of Embodiment A7 wherein the fluorine content of a halogenated hydrocarbon compound or a hydrocarbon compound is increased by reacting said compound with HF and Cl₂ in the presence of said catalyst composition.

Embodiment A10. The process of Embodiment A7 wherein the fluorine distribution in a halogenated hydrocarbon compound is changed by isomerizing said halogenated hydrocarbon compound in the presence of said catalyst composition.

Embodiment A11. The process of Embodiment A7 wherein the fluorine distribution in a halogenated hydrocarbon compound is changed by disproportionating said halogenated hydrocarbon compound in the presence of said catalyst composition.

Embodiment A12. The process of Embodiment A7 wherein the fluorine content of a halogenated hydrocarbon compound is decreased by dehydrofluorinating said halogenated hydrocarbon compound in the presence of said catalyst composition.

Embodiment A13. The process of Embodiment A7 wherein the fluorine content of a halogenated hydrocarbon compound is decreased by reacting said halogenated hydrocarbon compound with HCl in the vapor phase the presence of said catalyst composition.

Embodiment A14. A method for preparing the catalyst composition of Embodiment A1, comprising (a) co-precipitating a solid by adding ammonium hydroxide to an aqueous solution of a soluble modifier metal salts and a soluble chromium salt that contains at least three moles of nitrate per mole of chromium in the solution and has a modifier metal content of from about 0.05 atom % to about 10 atom % of the total content of modifier metal and chromium in the solution, to form an aqueous mixture containing co-precipitated solid; (b) drying said co-precipitated solid formed in (a); and (c) calcining said dried solid formed in (b) in an atmosphere containing at least 10% oxygen by volume.

Embodiment A15. The method of Embodiment A14 further comprising treating a calcined solid formed in (c) with a fluorinating agent to form a catalyst composition comprising chromium, oxygen, modifier metals, and fluorine as essential elements.

Embodiment A16. A method for preparing the catalyst composition of Embodiment A1, comprising (a) impregnating solid chromium oxide with a solution of a soluble modifier metal salts; (b) drying the impregnated chromium oxide prepared in (a); and (c) calcining the dried solid.

Embodiment A17. The method of Embodiment A16 further comprising treating a calcined solid formed in (c) with a fluorinating agent to form a catalyst composition comprising chromium, oxygen, modifier metals, and fluorine as essential elements.

Embodiment A18. A method for preparing the catalyst composition of Embodiment A1, comprising mixing multiple compositions, each comprising chromium, oxygen, and at least one modifier metal.

Embodiment A19. The method of Embodiment A18 further comprising treating the mixture of multiple compositions with a fluorinating agent to form a catalyst composition comprising chromium, oxygen, modifier metals, and fluorine as essential elements.

### EXAMPLES

### Catalyst Preparations

### Preparation Example A1

### Preparation of 95 Atom % Chromium/2.5 Atom % Gold/2.5 Atom % Silver Catalyst (400° C.) by Co-Precipitation

A two liter plastic beaker equipped with a pH probe and mechanical stirrer was charged with 800 mL of deionized water, 285.1 g of Cr(NO₃)₃[9(H₂O)], 16.1 g of gold solution (HAuCl₄, 23 weight % Au) and 3.2 g of AgNO₃ with stirring until dissolution was complete. To the stirred solution was slowly added a 50/50 mixture of ammonium hydroxide to raise the pH from 1.98 to 8.0. The resulting slurry was stirred at room temperature overnight. It was then dried at 110° C. to 120° C. in air for about 48 hours. The resulting solid was spread on a shallow pan and heated in air at the rate of 5° C./minute to bring the temperature to 400° C. and maintained at 400° C. for about 24 hours in air. The calcined solid was pressed into disks, broken up and sieved to provide a −12 to +20 mesh (1.68 to 0.84 mm) fraction that was used in Examples A1 and A6.

### Preparation Example A2

### Preparation of 95 Atom % Chromium/2.5 Atom % Gold/2.5 Atom % Silver Catalyst (900° C.) by Co-Precipitation

Preparation Example A1 was substantially repeated, except that the resulting solid was calcined in air at a final temperature of 900° C. The calcined solid was pressed into disks, broken up and sieved to provide a to +20 mesh (1.68 to 0.84 mm) fraction that was used in Examples A2 and A7.

### Preparation Example A3

### Preparation of 95 Atom % Chromium/2.5 Atom % Gold/2.5 Atom % Palladium Catalyst (400° C.) by Co-Precipitation

Preparation Example A1 was substantially repeated, using 285.2 g of Cr(NO₃)₃[9(H₂O)], 16.0 g of gold solution (HAuCl₄, 23 weight % Au) and 151.9 g of an aqueous palladium solution (1.31% Pd). The resulting solid was calcined in air at a final temperature of 400° C. The calcined solid was pressed into disks, broken up and sieved to provide a −12 to +20 mesh (1.68 to 0.84 mm) fraction that was used in Examples A3 and A8.

### Preparation Example A4

### Preparation of 95 Atom % Chromium/2.5 Atom % Palladium/2.5 Atom % Silver Catalyst (400° C.) by Co-Precipitation

Preparation Example A1 was substantially repeated, using 285.2 g of Cr(NO₃)₃[9(H₂O)], 3.2 g of AgNO₃ and 117.1 g of an aqueous palladium solution (1.70% Pd). The resulting solid was calcined in air at a final temperature of 400° C. The calcined solid was pressed into disks, broken up and sieved to provide a −12 to +20 mesh (1.68 to 0.84 mm) fraction that was used in Examples A4 and A9.

### Preparation Example A5

### Preparation of 95 Atom % Chromium/2.5 Atom % Palladium/2.5 Atom % Silver Catalyst (900° C.) by Co-Precipitation

Preparation Example A4 was substantially repeated, using 285.2 g of Cr(NO₃)₃[9(H₂O)], 3.2 g of AgNO₃ and 117.1 g of an aqueous palladium solution (1.70% Pd). The resulting solid was calcined in air at a final temperature of 900° C. The calcined solid was pressed into disks, broken up and sieved to provide a −12 to +20 mesh (1.68 to 0.84 mm) fraction that was used in Examples A5 and A10.

### Examples A1-A10

### General Procedure for Fluorination and Chlorofluorination

A weighed quantity of pelletized catalyst was placed in a ⅝ inch (1.58 cm) diameter Inconel™ nickel alloy reactor tube heated in a fluidized sand bath. The tube was heated from 50° C. to 175° C. in a flow of nitrogen (50 cc/min; 8.3(10)⁻⁷ m³/sec) over the course of about one hour. HF was then admitted to the reactor at a flow rate of 50 cc/min (8.3(10)⁻⁷ m³/sec). After 0.5 to 2 hours the nitrogen flow was decreased to 20 cc/min (3.3(10)⁻⁷ m³/sec) and the HF flow increased to 80 cc/min (1.3(10)⁻⁶ m³/sec); this flow was maintained for about 1 hour. The reactor temperature was then gradually increased to 400° C. over 3 to 5 hours. At the end of this period, the HF flow was stopped and the reactor cooled to the desired operating temperature under 20 sccm (3.3(10)⁻⁷ m³/sec) nitrogen flow. CFC-1213xa was fed from a pump to a vaporizer maintained at about 118° C. For fluorinations, the CFC-1213xa vapor was combined with the appropriate molar ratios of HF in a 0.5 inch (1.27 cm) diameter Monel™ nickel alloy tube packed with Monel™ turnings. The mixture of reactants then entered the reactor. For chlorofluorinations, the CFC-1213xa vapor was combined with the appropriate molar ratios of HF and chlorine prior to entering the reactor. The reactions were conducted at a nominal pressure of one atmosphere. Analytical data for identified compounds is given in units of GC area %.

### General Procedure for Fluorocarbon Product Analysis

The following general procedure is illustrative of the method used for analyzing the products of fluorination and chlorofluorination reactions. Part of the total reactor effluent was sampled on-line for organic product analysis using a gas chromatograph equipped a mass selective detector (GC-MS). The gas chromatography was accomplished with a 20 ft. (6.1 m) long×⅛ in. (0.32 cm) diameter tubing containing Krytox® perfluorinated polyether on an inert carbon support. The helium flow was 30 mL/min (5.0(10)⁻⁷ m³/sec). Gas chromatographic conditions were 60° C. for an initial hold period of three minutes followed by temperature programming to 200° C. at a rate of 6° C./minute.

The bulk of the reactor effluent containing organic products and also inorganic acids such as HCl and HF was treated with aqueous caustic prior to disposal.

### Legend

| | |
|---|---|
| 214ab is CF₃CCl₂CCl₂F | 215aa is CF₃CCl₂CClF₂ |
| 215bb is CCl₂FCClFCF₃ | 216aa is CF₃CCl₂CF₃ |
| 216ba is CClF₂CClFCF₃ | 217ba is CF₃CClFCF₃ |
| 225da is CF₃CHClCClF₂ | 226da is CF₃CHClCF₃ |
| 1213xa is CF₃CCl═CCl₂ | 1214 is C₃Cl₂F₄ |
| 1215xc is CF₃CCl═CF₂ | |

### Examples A1-A5

### Fluorination of CFC-1213xa

The fluorination of CFC-1213xa was carried out at various temperatures using the indicated weights of catalysts prepared according to Catalyst Preparation Examples A1-A5. The molar ratio of HF to 1213xa was 20:1 for all Examples and the contact time was 5 seconds. The analytical results are summarized in Table A1. Small quantities of other compounds, not summarized in Table A1 were also present.

TABLE A1

| | T | Cr/M1/M2 | Calcin T | Wt | | | | | | |
|---|---|---|---|---|---|---|---|---|---|---|
| Ex | (° C.) | (atom %) | (° C.) | (g) | 1215xc | 226da | 216aa | 1214xb | 225da | 1213xa |
| A1 | 250 | Cr/Au/Ag | 400 | 12.4 | 6.3 | 0.2 | 0.3 | 9.3 | 0.9 | 75.7 |
| | 275 | 95/2.5/2.5 | | | 27.2 | 1.3 | 1.5 | 11.3 | 1.5 | 53.1 |
| | 300 | | | | 48.7 | 2.8 | 2.8 | 13.2 | 1.6 | 28.4 |
| | 325 | | | | 61.3 | 3.5 | 3.7 | 14.7 | 1.6 | 14.1 |
| | 350 | | | | 70.4 | 5.1 | 2.5 | 14.1 | 1.5 | 6.2 |
| | 375 | | | | 73.4 | 5.0 | 1.0 | 14.4 | 1.2 | 4.8 |
| | 400 | | | | 74.0 | 4.1 | 0.9 | 15.3 | 0.9 | 4.6 |
| A2 | 300 | Cr/Au/Ag | 900 | 18.5 | 1.4 | ND | ND | 5.1 | ND | 93.4 |
| | 325 | 95/2.5/2.5 | | | 3.1 | 0.1 | ND | 7.5 | ND | 89.2 |
| | 350 | | | | 6.5 | 0.1 | ND | 10.3 | ND | 83.1 |
| | 375 | | | | 10.2 | 0.3 | ND | 11.9 | ND | 77.5 |
| | 400 | | | | 13.7 | 0.4 | ND | 11.7 | ND | 74.0 |
| A3 | 275 | Cr/Au/Pd | 400 | 11.7 | 3.2 | 93.4 | 1.1 | 0.6 | 1.0 | 0.3 |
| | 300 | 95/2.5/2.5 | | | 0.6 | 97.6 | 1.0 | 0.1 | 0.1 | ND |
| | 325 | | | | 0.7 | 97.2 | 0.8 | 0.2 | 0.1 | 0.1 |
| | 350 | | | | 1.7 | 94.5 | 1.1 | 0.5 | ND | 0.1 |
| | 375 | | | | 3.4 | 89.2 | 2.4 | 1.0 | ND | 0.3 |
| A4 | 275 | Cr/Ag/Pd | 400 | 12.7 | 21.2 | 0.9 | 1.3 | 13.1 | 1.6 | 57.6 |
| | 300 | 95/2.5/2.5 | | | 40.9 | 1.8 | 4.4 | 14.5 | 1.7 | 32.1 |
| | 325 | | | | 55.8 | 2.7 | 8.0 | 13.8 | 1.6 | 15.0 |
| | 350 | | | | 62.8 | 3.5 | 11.1 | 13.2 | 1.4 | 6.9 |
| | 375 | | | | 59.6 | 4.8 | 15.9 | 12.9 | 1.2 | 4.2 |
| | 400 | | | | 57.5 | 10.6 | 12.3 | 12.4 | 0.9 | 3.7 |
| A5 | 275 | Cr/Ag/Pd | 900 | 15.5 | 4.7 | 0.1 | ND | 9.7 | ND | 85.3 |
| | 300 | 95/2.5/2.5 | | | 23.3 | 0.4 | 0.2 | 15.3 | 1.0 | 59.8 |
| | 325 | | | | 43.4 | 0.9 | 0.4 | 17.7 | 1.2 | 36.4 |
| | 350 | | | | 59.8 | 1.8 | 1.3 | 18.4 | 1.4 | 16.9 |
| | 375 | | | | 67.5 | 8.7 | 2.0 | 14.2 | 1.3 | 5.7 |
| | 400 | | | | 64.8 | 14.1 | 2.7 | 11.5 | 0.7 | 4.6 |

Note:
ND means less than 0.1; M1, M2 mean modifier metals; Calcin. T means calcination temperature; Wt (g) means catalyst weight

### Examples A6-A10

### Chlorofluorination of CFC-1213xa

The chlorofluorination of CFC-1213xa was carried out at various temperatures using indicated weights of catalyst prepared according to Catalyst Preparation Examples A1-A5. The HF/1213xa/Cl2 molar ratio was 20/1/4 for all Examples and the contact time was 5 seconds. The analytical results are summarized in Table A2. Small quantities of other compounds, not summarized in Table A2, were also present.

TABLE A2

| | Reactor | Cr/M1/M2 | Calcin T | Wt | | | | | | | |
|---|---|---|---|---|---|---|---|---|---|---|---|
| Ex | Temp | (atom %) | (° C.) | (g) | 216aa | 216ba | 1214xb | 215aa | 215bb | 1213xa | 214ab |
| A6 | 280 | Cr/Au/Ag | 400 | 12.4 | 0.1 | ND | 8.4 | 2.5 | 14.6 | 56.4 | 16.5 |
| | 300 | 95/2.5/2.5 | | | 0.1 | ND | 8.5 | 2.6 | 15.3 | 55.6 | 16.6 |
| | 325 | | | | 0.1 | 0.1 | 9.2 | 3.9 | 17.7 | 46.5 | 21.2 |
| | 350 | | | | 0.6 | 0.4 | 7.3 | 12.0 | 29.9 | 19.6 | 28.3 |
| | 375 | | | | 2.4 | 1.7 | 3.4 | 30.6 | 33.2 | 4.1 | 22.6 |
| | 400 | | | | 5.9 | 4.4 | 1.0 | 48.3 | 28.4 | 0.6 | 9.5 |
| A7 | 300 | Cr/Au/Ag | | | ND | ND | 8.1 | 0.8 | 10.9 | 72.5 | 6.9 |
| | 325 | 95/2.5/2.5 | 900 | 18.5 | ND | ND | 5.6 | 1.0 | 6.5 | 74.6 | 11.7 |
| | 350 | | | | ND | 0.1 | 6.2 | 2.0 | 8.0 | 67.2 | 15.2 |
| | 375 | | | | 0.1 | 0.3 | 7.9 | 5.0 | 12.5 | 53.6 | 18.2 |
| | 400 | | | | 0.3 | 0.7 | 8.8 | 8.5 | 14.8 | 45.5 | 17.4 |
| A8 | 280 | Cr/Au/Pd | 400 | 11.7 | 2.7 | 1.1 | 2.0 | 31.4 | 32.1 | ND | 29.1 |
| | 300 | 95/2.5/2.5 | | | 4.1 | 2.6 | 1.0 | 44.4 | 31.7 | ND | 14.5 |
| | 325 | | | | 7.2 | 8.3 | 0.3 | 52.5 | 27.3 | ND | 2.3 |
| | 350 | | | | 12.6 | 18.5 | 0.1 | 50.2 | 15.9 | ND | 0.2 |
| | 375 | | | | 20.8 | 28.2 | 0.0 | 43.7 | 4.3 | ND | 0.1 |
| | 400 | | | | 31.5 | 30.4 | 0.0 | 34.0 | 0.5 | ND | 0.1 |
| A9 | 280 | Cr/Ag/Pd | 400 | 12.7 | 9.7 | 3.6 | 4.5 | 14.6 | 45.2 | 0.3 | 17.0 |
| | 300 | 95/2.5/2.5 | | | 11.2 | 3.1 | 2.8 | 17.4 | 44.8 | 0.3 | 16.8 |
| | 325 | | | | 12.7 | 3.4 | 0.8 | 22.5 | 43.3 | 0.2 | 13.8 |
| | 350 | | | | 15.3 | 5.7 | 0.6 | 26.8 | 41.4 | 0.1 | 6.7 |
| | 375 | | | | 17.0 | 7.8 | 0.4 | 34.8 | 33.4 | 0.1 | 3.8 |
| | 400 | | | | 20.3 | 13.6 | 0.2 | 36.9 | 25.0 | 0.0 | 1.0 |
| A10 | 280 | Cr/Ag/Pd | 900 | 15.5 | 0.8 | 0.2 | 14.7 | 9.3 | 42.0 | 20.7 | 9.1 |
| | 300 | 95/2.5/2/5 | | | 1.2 | 0.2 | 8.0 | 11.7 | 38.7 | 18.0 | 19.4 |
| | 325 | | | | 2.6 | 0.4 | 4.1 | 19.1 | 44.3 | 6.7 | 20.0 |
| | 350 | | | | 6.0 | 1.3 | 1.3 | 25.6 | 48.3 | 0.7 | 14.2 |
| | 375 | | | | 9.7 | 2.3 | 1.0 | 27.6 | 44.7 | 0.4 | 11.6 |
| | 400 | | | | 15.9 | 4.0 | 0.8 | 29.1 | 39.0 | 0.3 | 7.8 |

Note:
ND means less than 0.1; M1, M2 mean modifier metals; Calcin. T means calcination temperature; Wt (g) means catalyst weight

The examples above illustrate use of the catalysts of this invention to increase the fluorine content of a compound. Using the catalysts of this invention, the fluorine distribution in a halogenated hydrocarbon compound may be changed by isomerization or disproportionation or the fluorine content of a compound may be decreased by dehydrofluorination or by reaction with hydrogen chloride in a manner analogous to the teachings of International Publication No. WO 2004/018093 A2, which is incorporated herein by reference.

### B.

Invention Category B of this application provides a process for the preparation of CF₃CH₂CHF₂ (HFC-245fa) and CF₃CHFCH₂F (HFC-245eb).

In step (a) of the process of this invention, one or more halopropene compounds of the formula CX₃CCl═CClX, wherein each X is independently selected from the group consisting of F and Cl, are reacted with chlorine (Cl₂) and hydrogen fluoride (HF) to produce a product mixture comprising CF₃CCl₂CClF₂ (CFC-215aa) and CF₃CClFCCl₂F (CFC-215bb). Accordingly, this invention provides a process for the preparation of mixtures of CF₃CCl₂CClF₂ (CFC-215aa) and CF₃CClFCCl₂F (CFC-215bb) from readily available starting materials.

Suitable starting materials for the process of this invention include E- and Z-CF₃CCl═CClF (CFC-1214xb), CF₃CCl═CCl₂ (CFC-1213xa), CClF₂CCl═CCl₂ (CFC-1212xa), CCl₂FCCl═CCl₂ (CFC-1211xa), and CCl₃CCl═CCl₂ (hexachloropropene, HCP), or mixtures thereof.

Due to their availability, CF₃CCl═CCl₂ (CFC-1213xa) and CCl₃CCl═CCl₂ (hexachloropropene, HCP) are the preferred starting materials for the process of the invention.

Preferably, the reaction of HF and Cl₂ with CX₃CCl═CClX is carried out in the vapor phase in a heated tubular reactor. A number of reactor configurations are possible, including vertical and horizontal orientation of the reactor and different modes of contacting the halopropene starting material(s) with HF and chlorine. Preferably the HF and chlorine are substantially anhydrous.

In one embodiment of step (a), the halopropene starting material(s) are fed to the reactor containing the chlorofluorination catalyst. The halopropene starting material(s) may be initially vaporized and fed to the reaction zone as gas(es).

In another embodiment of step (a), the halopropene starting material(s) may be contacted with HF in a pre-reactor (i.e. prior to contacting the chlorofluorination catalysts). The pre-reactor may be empty (i.e., unpacked), but is preferably filled with a suitable packing such as Monel™ or Hastelloy™ nickel alloy turnings or wool, or other material inert to HCl and HF, that allows for efficient mixing of CX₃CCl═CClX and HF vapor.

If the halopropene starting material(s) are fed to the pre-reactor as liquid(s), it is preferable for the pre-reactor to be oriented vertically with CX₃CCl═CClX entering the top of the reactor and pre-heated HF vapor introduced at the bottom of the reactor.

Suitable temperatures for the pre-reactor are within the range of from about 80° C. to about 250° C., preferably from about 100° C. to about 200° C. Under these conditions, for example, hexachloropropene is converted to a mixture containing predominantly CFC-1213xa. The feed rate of the starting material is determined by the length and diameter of the reactor, reactor temperature, and the degree of fluorination desired in the pre-reactor. Slower feed rates at a given temperature will increase contact time and tend to increase the amount of conversion of the starting material and increase the degree of fluorination of the products.

The term “degree of fluorination” means the extent to which fluorine atoms replace chlorine substituents in the CX₃CCl═CClX starting materials. For example, CF₃CCl═CClF represents a higher degree of fluorination than CClF₂CCl═CCl₂ and CF₃CCl₂CF₃ represents a higher degree of fluorination than CClF₂CCl₂CF₃.

The molar ratio of HF fed to the pre-reactor, or otherwise to the reaction zone of step (a), to halopropene starting material fed in step (a), is typically from about stoichiometric to about 50:1. The stoichiometric ratio depends on the average degree of fluorination of the halopropene starting material(s) and is typically based on formation of C₃Cl₃F₅ isomers. For example, if the halopropene is HCP, the stoichiometric ratio of HF to HCP is 5:1; if the halopropene is CFC-1213xa, the stoichiometric ratio of HF to CFC-1213xa is 2:1. Preferably, the molar ratio of HF to halopropene starting material is from about twice the stoichiometric ratio (based on formation of C₃Cl₃F₅ isomers) to about 30:1. Higher ratios of HF to halopropene are not particularly beneficial. Lower ratios result in reduced yields of C₃Cl₃F₅ isomers.

If the halopropene starting materials are contacted with HF in a pre-reactor, the effluent from the pre-reactor is then contacted with chlorine in the reaction zone of step (a).

In another embodiment of the invention, the halopropene starting material(s) may be contacted with Cl₂ and HF in a pre-reactor (i.e. prior to contacting the chlorofluorination catalyst). The pre-reactor may be empty (i.e., unpacked) but is preferably filled with a suitable packing such as Monel™ or Hastelloy™ nickel alloy turnings or wool, activated carbon, or other material inert to HCl, HF, and Cl₂ that allows for efficient mixing of CX₃CCl═CClX, HF, and Cl₂.

Typically, at least a portion of the halopropene starting material(s) react(s) with Cl₂ and HF in the pre-reactor by addition of Cl₂ to the olefinic bond to give a saturated halopropane as well as by substitution of at least a portion of the Cl substituents in the halopropropane and/or halopropene by F. Suitable temperatures for the pre-reactor in this embodiment of the invention are within the range of from about 80° C. to about 250° C., preferably from about 100° C. to about 200° C. Higher temperatures result in greater conversion of the halopropene(s) entering the reactor to saturated products and greater degrees of halogenation and fluorination in the pre-reactor products.

The term “degree of halogenation” means the extent to which hydrogen substituents in a halocarbon have been replaced by halogen and the extent to which carbon-carbon double bonds have been saturated with halogen. For example, CF₃CCl₂CClF₂ has a higher degree of halogenation than CF₃CCl═CCl₂. Also, CF₃CCl₂CClF₂ has a higher degree of halogenation than CF₃CHClCClF₂.

The molar ratio of Cl₂ to halopropene starting material(s) is typically from about 1:1 to about 10:1, and is preferably from about 1:1 to about 5:1. Feeding Cl₂ at less than a 1:1 ratio will result in the presence of relatively large amounts of unsaturated materials and hydrogen-containing side products in the reactor effluent.

In a preferred embodiment of step (a), the halopropene starting materials are vaporized, preferably in the presence of HF, and contacted with HF and Cl₂ in a pre-reactor and then contacted with the chlorofluorination catalyst. If the preferred amounts of HF and Cl₂ are fed to the pre-reactor, additional HF and Cl₂ are not required in the reaction zone.

Suitable temperatures in the reaction zone(s) of step (a) are within the range of from about 200° C. to about 400° C., preferably from about 250° C. to about 350° C., depending on the desired conversion of the starting material and the activity of the catalyst. Reactor temperatures greater than about 350° C. may result in products having a degree of fluorination greater than five. In other words, at higher temperatures, substantial amounts of chloropropanes containing six or more fluorine substituents (e.g., CF₃CCl₂CF₃ or CF₃CClFCClF₂) may be formed. Reactor temperature below about 240° C. may result in a substantial yield of products with a degree of fluorination less than five (i.e., underfluorinates).

Suitable reactor pressures for vapor phase embodiments of this invention may be in the range of from about 1 to about 30 atmospheres. Reactor pressures of about 5 atmospheres to about 20 atmospheres may be advantageously employed to facilitate separation of HCl from other reaction products.

The chlorofluorination catalysts used in step (a) of this invention comprise chromium, oxygen and modifier metals (e.g., modifier metal-containing chromium oxide) and contain from about 0.05 atom % to about 10 atom % modifier metals based on the total amount of modifier metals and chromium in the catalyst composition. Of note are compositions comprising silver and gold wherein the mole ratio of silver to gold is from about 10:1 to about 1:10. Also of note are compositions comprising silver and palladium wherein the mole ratio of silver to palladium is from about 10:1 to about 1:10. Also of note are compositions comprising palladium and gold wherein the mole ratio of palladium to gold is from about 10:1 to about 1:10.

In one embodiment of this invention, the catalyst composition comprises alpha-chromium oxide (i.e., α-Cr₂O₃) and metallic gold (i.e., gold in the zero oxidation state) and metallic silver (i.e., silver in the zero oxidation state). In another embodiment of this invention, the catalyst composition comprises alpha-chromium oxide (i.e., α-Cr₂O₃) and metallic gold (i.e., gold in the zero oxidation state) and palladium. In another embodiment of this invention, the catalyst composition comprises alpha-chromium oxide (i.e., α-Cr₂O₃) and metallic silver (i.e., silver in the zero oxidation state) and palladium. In another embodiment of this invention, the catalyst composition comprises alpha-chromium oxide (i.e., α-Cr₂O₃), metallic gold (i.e., gold in the zero oxidation state), metallic silver (i.e., silver in the zero oxidation state), and palladium. Of note are embodiments wherein at least 50 weight % of the chromium component is present as alpha-chromium oxide. Also of note are embodiments wherein the gold component consists essentially of metallic gold having an average particle size of from about 1 nanometer to about 500 nanometers. In certain embodiments of this invention, particles of metallic gold and at least one of palladium and metallic silver are dispersed in a matrix comprising chromium oxide. In certain embodiments of this invention, particles of metallic silver and palladium are dispersed in a matrix comprising chromium oxide.

In certain embodiments of this invention, particles of metallic gold and at least one of palladium and metallic silver are supported on a chromium oxide support. In some embodiments, particles of metallic silver and palladium are supported on a chromium oxide support.

The catalyst compositions of this invention may further comprise fluorine as an essential constituent element.

The amount of modifier metals relative to the total amount of chromium and modifier metals in the catalyst compositions used for the chlorofluorination reaction is preferably from about 0.5 atom % to about 5 atom %.

The modifier metal-containing chromium oxide catalysts used in the present invention can be formed into various shapes such as pellets, granules, and extrudates for use in packing reactors. They can also be used in powder forms.

Further information on catalyst compositions comprising chromium, oxygen, and at least two modifier metals selected from the group consisting of gold, silver, and palladium as essential constituent elements useful for this invention (including embodiments further comprising fluorine) is provided in Invention Category A above and in U.S. Patent Applications 60/903,218 [FL 1356 US PRV] filed Feb. 23, 2007, and 60/927,846 [FL 1356 US PRV1] filed May 4, 2007, hereby incorporated herein by reference in their entirety.

The catalyst compositions used in step (a) of this invention may further comprise one or more additives in the form of metal compounds. Such additives may alter the selectivity and/or activity of the modifier metal-containing chromium oxide catalyst compositions or the fluorinated modifier metal-containing chromium oxide catalyst compositions. Suitable additives can be selected from the group consisting of the fluorides, oxides, and oxyfluoride compounds of Mg, Ca, Sc, Y, La, Ti, Zr, W, Mn, Re, Fe, Ru, Co, Rh, Ir, Ni, Pt, Ce, and Zn.

The total content of the additive(s) in the catalyst compositions used in step (a) of the present invention may be from about 0.05 weight % to about 10 weight % based on the total metal content of the catalyst compositions. The additives may be incorporated into the catalyst compositions by standard procedures such as by impregnation or during co-precipitation of the modifier metal and chromium salts.

The catalyst compositions used in step (a) of the present invention can be treated with a fluorinating agent to form catalyst compositions comprising chromium, oxygen, modifier metals and fluorine as essential elements. Typically, prior to use as catalysts, the catalyst compositions are pre-treated with a fluorinating agent. Typically this fluorinating agent is HF though other materials may be used such as sulfur tetrafluoride, carbonyl fluoride, and fluorinated hydrocarbon compounds such as trichlorofluoromethane, dichlorodifluoromethane, chlorodifluoromethane, trifluoromethane, and 1,1,2-trichlorotrifluoroethane. This pretreatment can be accomplished, for example, by placing the catalyst composition in a suitable container which can also be the reactor to be used to perform the process in the present invention, and thereafter, passing HF over the calcined catalyst composition so as to partially saturate the catalyst composition with HF. This can be conveniently carried out by passing HF over the catalyst composition for a period of time, for example, about 0.1 to about 10 hours at a temperature of, for example, about 200° C. to about 450° C. Nevertheless, this pre-treatment is not essential.

Compounds that are produced in the chlorofluorination process in step (a) include the halopropanes CF₃CCl₂CClF₂ (CFC-215aa) and CF₃CClFCCl₂F (CFC-215bb).

Halopropane by-products that have a higher degree of fluorination than CFC-215aa and CFC-215bb that may be produced in step (a) include CF₃CCl₂CF₃ (CFC-216aa), CF₃CClFCClF₂ (CFC-216ba), CF₃CF₂CCl₂F (CFC-216cb), CF₃CClFCF₃ (CFC-217ba), and CF₃CHClCF₃ (HCFC-226da).

Halopropane by-products that may be formed in step (a) which have lower degrees of fluorination than CFC-215aa and CFC-215bb include CF₃CCl₂CCl₂F (HCFC-214ab) and CF₃CCl₂CCl₃ (HCFC-213ab).

Halopropene by-products that may be formed in step (a) include CF₃CCl═CF₂ (CFC-1215xc), E- and Z-CF₃CCl═CClF (CFC-1214xb), and CF₃CCl═CCl₂ (CFC-1213xa).

Prior to step (b), CF₃CCl₂CClF₂ (CFC-215aa) and CF₃CClFCCl₂F (CFC-215bb) (and optionally HF) from the effluent from the reaction zone in step (a), are typically separated from lower boiling components of the effluent (which typically comprise HCl, Cl₂, HF, overfluorinated products such as C₃ClF₇ and C₃Cl₂F₆ isomers) and the underhalogenated and underfluorinated components of the effluent (which typically comprise C₃ClF₅ and C₃Cl₂F₄, CFC-214ab, CFC-1212xb and CFC-1213xa). Underfluorinated and underhalogenated components (e.g., CFC-214ab, CFC-1212xb, and CFC-1213xa) may be returned to step (a).

In one embodiment of the present invention, the overfluorinated components include CFC-216aa, and CFC-216ba, which are further reacted with hydrogen (H₂), optionally in the presence of HF, to produce 1,1,1,3,3,3-hexafluoropropane (HFC-236fa), and at least one of 1,1,1,2,3,3-hexafluoropropane (HFC-236ea) and hexafluoropropene as disclosed in U.S. Patent Application 60/927,847 [FL-1367 US PRV] filed May 4, 2007 and hereby incorporated herein by reference.

In another embodiment of the invention, the reactor effluent from step (a) may be delivered to a first distillation column in which HCl and any HCl azeotropes are removed from the top of column while the higher boiling components are removed at the bottom of the column. The products recovered at the bottom of the first distillation column are then delivered to a second distillation column in which HF, Cl₂, CF₃CCl₂CF₃ (CFC-216aa), CF₃CClFCClF₂ (CFC-216ba), CF₃CF₂CCl₂F (CFC-216cb), CF₃CClFCF₃ (CFC-217ba), and CF₃CHClCF₃ (HCFC-226da) and their HF azeotropes are recovered at the top of the column and CFC-215aa and CFC-215bb, and any remaining HF and the higher boiling components are removed from the bottom of the column. The products recovered from the bottom of the second distillation column may then be delivered to a further distillation column to separate the underfluorinated by-products and intermediates to isolate CFC-215aa and CFC-215bb.

Optionally, after distillation and separation of HCl from the reactor effluent of step (a), the resulting mixture of HF and halopropanes and halopropenes may be delivered to a decanter controlled at a suitable temperature to permit separation of a liquid HF-rich phase and a liquid organic-rich phase. The organic-rich phase may then be processed to isolate the CFC-215aa and CFC-215bb. The HF-rich phase may then be recycled to the reactor of step (a), optionally after removal of any organic components. The decantation step may be used at other points in the CFC-215aa/CFC-215bb separation scheme where HF is present.

In step (b) of the process of this invention, CF₃CCl₂CClF₂ (CFC-215aa) and CF₃CClFCCl₂F (CFC-215bb) produced in step (a) are reacted with hydrogen (H₂) in a second reaction zone.

In one embodiment of step (b), a mixture comprising CFC-215aa and CFC-215bb is delivered in the vapor phase, along with hydrogen (H₂), to a reactor containing a hydrogenation catalyst. Hydrogenation catalysts suitable for use in this embodiment include catalysts comprising at least one metal selected from the group consisting of iron, ruthenium, rhodium, iridium, palladium, and platinum. Said catalytic metal component is typically supported on a carrier such as carbon or graphite. Of note are carbon supported catalysts in which the carbon support has been washed with acid and has an ash content below about 0.1% by weight. Hydrogenation catalysts supported on low ash carbon are described in U.S. Pat. No. 5,136,113, the teachings of which are incorporated herein by reference. Of particular note are catalysts of palladium supported on carbon. The hydrogenation of CFC-215aa and CFC-215bb to produce HFC-245fa and HFC-245eb is disclosed in International Publication No. WO 2005/037743 A1, which is incorporated herein by reference.

The supported metal catalysts may be prepared by conventional methods known in the art such as by impregnation of the carrier with a soluble salt of the catalytic metal (e.g., palladium chloride or rhodium nitrate) as described by Satterfield on page 95 of Heterogenous Catalysis in Industrial Practice, 2^{nd} edition (McGraw-Hill, New York, 1991). The concentration of the catalytic metal(s) on the support is typically in the range of about 0.1% by weight of the catalyst to about 5% by weight.

The relative amount of hydrogen contacted with CFC-215aa and CFC-215bb (i.e., trichloropentafluoropropanes, C₃Cl₃F₅ isomers) in the presence of a hydrogenation catalyst is typically from about 0.5 mole of H₂ per mole of trichloropentafluoropropane isomer to about 10 moles of H₂ per mole of trichloropentafluoropropane isomer, preferably from about 3 moles of H₂ per mole of trichloropentafluoropropane isomer to about 8 moles of H₂ per mole of trichloropentafluoropropane isomer.

Suitable temperatures for the catalytic hydrogenation are typically in the range of from about 100° C. to about 350° C., preferably from about 125° C. to about 300° C. Temperatures above about 350° C. tend to result in defluorination side reactions; temperatures below about 125° C. will result in incomplete substitution of Cl for H in the C₃Cl₃F₅ starting materials. The reactions are typically conducted at atmospheric pressure or superatmospheric pressure.

The effluent from the step (b) reaction zone typically includes HCl, unreacted hydrogen, CF₃CH₂CHF₂ (HFC-245fa), CF₃CHFCH₂F (HFC-245eb), lower boiling by-products (typically including CF₃CH═CF₂ (HFC-1225zc), E- and Z-CF₃CH═CHF (HFC-1234ze), CF₃CF═CH₂ (HFC-1234yf), CF₃CH₂CF₃ (HFC-236fa), CF₃CHFCH₃ (HFC-254eb), and/or CF₃CH₂CH₃ (HFC-263fb)) and higher boiling by-products and intermediates (typically including CF₃CH₂CH₂Cl (HCFC-253fb), CF₃CHFCH₂Cl (HCFC-244eb), CF₃CClFCH₂F (HCFC-235bb), CF₃CHClCHF₂ (HCFC-235da), CF₃CHClCClF₂ (HCFC-225da), and/or CF₃CClFCHClF (HCFC-225ba diasteromers)) as well as any HF carried over from step (a) or step (b).

In step (c), the desired products are recovered. The HFC-245fa and HFC-245eb are typically separated from the lower boiling products and higher boiling products by conventional means (e.g., distillation). Partially chlorinated by-products such as HCFC-235da, HCFC-235bb, HCFC-225ba, and HCFC-225da may be recycled back to step (b).

In one embodiment of the present invention, CF₃CH₂CHF₂ (HFC-245fa) and CF₃CHFCH₂F (HFC-245eb) produced in step (b), are dehydrofluorinated to produce a product comprising CF₃CH═CHF (HFC-1234ze) and CF₃CF═CH₂ (HFC-1234yf) and at least one compound selected from the group consisting of CF₃CH═CHF and CF₃CF═CH₂ is recovered as disclosed in U.S. Patent Application 60/927,838 [FL-1366 US PRV] filed May 4, 2007 and hereby incorporated herein by reference.

HFC-245fa, HFC-245eb and/or mixtures of them may be used as refrigerants, foam expansion agents or chemical intermediates. Of note is a foam expansion agent comprising a mixture of 1,1,1,3,3-pentafluoropropane and 1,1,1,2,3-pentafluoropropane produced in accordance with this invention.

Embodiments of this invention include, but are not limited to:

Embodiment B1. A process for making CF₃CH₂CHF₂ and CF₃CHFCH₂F, comprising (a) reacting HF, Cl₂, and at least one halopropene of the formula CX₃CCl═CClX, wherein each X is independently selected from the group consisting of F and Cl, to produce a product comprising CF₃CCl₂CClF₂ and CF₃CClFCCl₂F, wherein said CF₃CCl₂CClF₂ and CF₃CClFCCl₂F are produced in the presence of a catalyst composition comprising chromium, oxygen, and at least two modifier metals selected from the group consisting of gold, silver, and palladium as essential constituent elements, wherein the total amount of modifier metals in said catalyst composition is from about 0.05 atom % to about 10 atom % based on the total amount of chromium and modifier metals in the catalyst composition; (b) reacting CF₃CCl₂CClF₂ and CF₃CClFCCl₂F produced in (a) with H₂, to produce a product comprising CF₃CH₂CHF₂ and CF₃CHFCH₂F; and (c) recovering CF₃CH₂CHF₂ and CF₃CHFCH₂F from the product produced in (b).

Embodiment B2. The process of Embodiment B1 wherein the halopropene reactant is contacted with Cl₂ and HF in a pre-reactor.

Embodiment B3. The process of Embodiment B1 wherein the halopropene reactant is contacted with HF in a pre-reactor.

Embodiment B4. The process of Embodiment B1 wherein the reaction of (b) is conducted in a reaction zone at a temperature of from about 100° C. to about 350° C. containing a hydrogenation catalyst.

Embodiment B5. The process of Embodiment B1 wherein the amount of modifier metals relative to the total amount of chromium and modifier metals in the catalyst composition is from about 0.5 atom % to about 5 atom %.

Embodiment B6. The process of Embodiment B1 wherein the catalyst composition further comprises fluorine as an essential constituent element.

Embodiment B7. The process of Embodiment B1 wherein the catalyst composition comprises alpha-chromium oxide, metallic gold and metallic silver.

Embodiment B8. The process of Embodiment B1 wherein the catalyst composition comprises alpha-chromium oxide, metallic gold and palladium.

Embodiment B9. The process of Embodiment B1 wherein the catalyst composition comprises alpha-chromium oxide, metallic silver and palladium.

Embodiment B10. The process of Embodiment B1 wherein the catalyst composition comprises alpha-chromium oxide, metallic gold, metallic silver and palladium.

Embodiment B11. The process of Embodiment B1 wherein the catalyst composition comprises silver and gold and the mole ratio of silver to gold is from about 10:1 to about 1:10.

Embodiment B12. The process of Embodiment B1 wherein the catalyst composition comprises silver and palladium and the mole ratio of silver to palladium is from about 10:1 to about 1:10.

Embodiment B13. The process of Embodiment B1 wherein the catalyst composition comprises palladium and gold and the mole ratio of palladium to gold is from about 10:1 to about 1:10.

### Examples

Reference is made to Examples A6-A10 in Invention Category A above for the chlorofluorination of CFC-1213xa.

Examination of the data shown in Table A2 above show that the amount of CFC-215aa and CFC-215bb can be maximized relative to CFC-216aa and CFC-216ba by controlling the operational variables by using the catalysts of this invention. The CFC-215aa and CFC-215bb produced above may be hydrogenated to produce HFC-245fa and HFC-245eb, respectively, in a manner analogous to the teachings of International Publication No. WO 2005/037743 A1. The CF₃CH₂CHF₂ and CF₃CHFCH₂F may be recovered by procedures known to the art.

### C.

Invention Category C of this application provides a process for the manufacture of CF₃CH═CHF (HFC-1234ze) and/or CF₃CF═CH₂ (HFC-1234yf). The HFC-1234ze and HFC-1234yf may be recovered as individual products and/or as one or more mixtures of the two products. HFC-1234ze may exist as one of two configurational isomers, E or Z. HFC-1234ze as used herein refers to the isomers, E-HFC-1234ze or Z-HFC-1234ze, as well as any combinations or mixtures of such isomers.

In step (a) of the process of this invention, one or more halopropene compounds of the formula CX₃CCl═CClX, wherein each X is independently selected from the group consisting of F and Cl, are reacted with chlorine (Cl₂) and hydrogen fluoride (HF) to produce a product mixture comprising CF₃CCl₂CClF₂ (CFC-215aa) and CF₃CClFCCl₂F (CFC-215bb). Accordingly, this invention provides a process for the preparation of mixtures of CF₃CCl₂CClF₂ (CFC-215aa) and CF₃CClFCCl₂F (CFC-215bb) from readily available starting materials.

Suitable halopropene starting materials CX₃CCl═CClX for the process of this invention include E- and Z-CF₃CCl═CClF (CFC-1214xb), CF₃CCl═CCl₂ (CFC-1213xa), CClF₂CCl═CCl₂ (CFC-1212xa), CCl₂FCCl═CCl₂ (CFC-1211xa), and CCl₃CCl═CCl₂ (hexachloropropene, HCP), or mixtures thereof.

Due to their availability, CF₃CCl═CCl₂ (CFC-1213xa) and CCl₃CCl═CCl₂ (hexachloropropene, HCP) are the preferred starting materials for the process of the invention.

Preferably, the reaction of HF and Cl₂ with CX₃CCl═CClX is carried out in the vapor phase in a heated tubular reactor. A number of reactor configurations are possible, including vertical and horizontal orientation of the reactor and different modes of contacting the halopropene starting material(s) with HF and chlorine. Preferably the HF and chlorine are substantially anhydrous.

In one embodiment of step (a), the halopropene starting material(s), HF and Cl₂ are fed to the reaction zone for contacting the chlorofluorination catalyst. The halopropene starting material(s) may be initially vaporized and fed to the reaction zone as gas(es).

In another embodiment of step (a), the halopropene starting material(s) may be contacted with HF in a pre-reactor (i.e. prior to contacting the chlorofluorination catalysts). The pre-reactor may be empty (i.e., unpacked), but is preferably filled with a suitable packing such as Monel™ or Hastelloy™ nickel alloy turnings or wool, (or other material inert to HCl and HF), which allows for efficient mixing of CX₃CCl═CClX and HF vapor.

If the halopropene starting material(s) are fed to the pre-reactor as liquid(s), it is preferable for the pre-reactor to be oriented vertically with CX₃CCl═CClX entering the top of the reactor and pre-heated HF vapor introduced at the bottom of the reactor.

Suitable temperatures for the pre-reactor are within the range of from about 80° C. to about 250° C., preferably from about 100° C. to about 200° C. Under these conditions, for example, hexachloropropene is converted to a mixture containing predominantly CFC-1213xa. The feed rate of the starting material is determined by the length and diameter of the reactor, reactor temperature, and the degree of fluorination desired in the pre-reactor. Slower feed rates at a given temperature will increase contact time and tend to increase the amount of conversion of the starting material and increase the degree of fluorination of the products.

The term “degree of fluorination” means the extent to which fluorine atoms replace chlorine substituents in the CX₃CCl═CClX starting materials. For example, CF₃CCl═CClF represents a higher degree of fluorination than CClF₂CCl═CCl₂ and CF₃CCl₂CF₃ represents a higher degree of fluorination than CClF₂CCl₂CF₃.

The molar ratio of HF fed to the pre-reactor, or otherwise to the reaction zone of step (a), to halopropene starting material fed in step (a), is typically from about stoichiometric to about 50:1. The stoichiometric ratio depends on the average degree of fluorination of the halopropene starting material(s) and is typically based on formation of C₃Cl₃F₅ isomers. For example, if the halopropene is HCP, the stoichiometric ratio of HF to HCP is 5:1; if the halopropene is CFC-1213xa, the stoichiometric ratio of HF to CFC-1213xa is 2:1. Preferably, the molar ratio of HF to halopropene starting material is from about twice the stoichiometric ratio (based on formation of C₃Cl₃F₅ isomers) to about 30:1. Higher ratios of HF to halopropene are not particularly beneficial. Lower ratios result in reduced yields of C₃Cl₃F₅ isomers.

If the halopropene starting materials are contacted with HF in a pre-reactor, the effluent from the pre-reactor is then contacted with chlorine in the reaction zone of step (a).

In another embodiment of step (a), the halopropene starting material(s) may be contacted with Cl₂ and HF in a pre-reactor (i.e. prior to contacting the chlorofluorination catalyst). The pre-reactor may be empty (i.e., unpacked) but is preferably filled with a suitable packing such as Monel™ or Hastelloy™ nickel alloy turnings or wool, activated carbon, or other material inert to HCl, HF, and Cl₂ that allows for efficient mixing of CX₃CCl═CClX, HF, and Cl₂.

Typically, at least a portion of the halopropene starting material(s) react(s) with Cl₂ and HF in the pre-reactor by addition of Cl₂ to the olefinic bond to give a saturated halopropane as well as by substitution of at least a portion of the Cl substituents in the halopropropane and/or halopropene by F. Suitable temperatures for the pre-reactor in this embodiment of the invention are within the range of from about 80° C. to about 250° C., preferably from about 100° C. to about 200° C. Higher temperatures result in greater conversion of the halopropene(s) entering the reactor to saturated products and greater degrees of halogenation and fluorination in the pre-reactor products.

The term “degree of halogenation” means the extent to which hydrogen substituents in a halocarbon have been replaced by halogen and the extent to which carbon-carbon double bonds have been saturated with halogen. For example, CF₃CCl₂CClF₂ has a higher degree of halogenation than CF₃CCl═CCl₂. Also, CF₃CCl₂CClF₂ has a higher degree of halogenation than CF₃CHClCClF₂.

The molar ratio of Cl₂ to halopropene starting material(s) is typically from about 1:1 to about 10:1, and is preferably from about 1:1 to about 5:1. Feeding Cl₂ at less than a 1:1 ratio will result in the presence of relatively large amounts of unsaturated materials and hydrogen-containing side products in the reactor effluent.

In a preferred embodiment of step (a), the halopropene starting materials are vaporized, preferably in the presence of HF, and contacted with HF and Cl₂ in a pre-reactor and then contacted with the chlorofluorination catalyst. If the preferred amounts of HF and Cl₂ are fed to the pre-reactor, additional HF and Cl₂ are not required in the reaction zone.

Suitable temperatures in the reaction zone(s) of step (a) are within the range of from about 200° C. to about 400° C., preferably from about 250° C. to about 350° C., depending on the desired conversion of the starting material and the activity of the catalyst. Reactor temperatures greater than about 350° C. may result in products having a degree of fluorination greater than five. In other words, at higher temperatures, substantial amounts of chloropropanes containing six or more fluorine substituents (e.g., CF₃CCl₂CF₃ or CF₃CClFCClF₂) may be formed. Reactor temperature below about 240° C. may result in a substantial yield of products with a degree of fluorination less than five (i.e., underfluorinates).

Suitable reactor pressures for vapor phase embodiments of this invention may be in the range of from about 1 to about 30 atmospheres. Reactor pressures of about 5 atmospheres to about 20 atmospheres may be advantageously employed to facilitate separation of HCl from other reaction products.

The chlorofluorination catalysts used in step (a) of this invention comprise chromium, oxygen and modifier metals (e.g., modifier metal-containing chromium oxide) and contain from about 0.05 atom % to about 10 atom % modifier metals based on the total amount of modifier metals and chromium in the catalyst composition. Of note are compositions comprising silver and gold wherein the mole ratio of silver to gold is from about 10:1 to about 1:10. Also of note are compositions comprising silver and palladium wherein the mole ratio of silver to palladium is from about 10:1 to about 1:10. Also of note are compositions comprising palladium and gold wherein the mole ratio of palladium to gold is from about 10:1 to about 1:10.

In one embodiment of this invention, the catalyst composition comprises alpha-chromium oxide (i.e., □-Cr₂O₃) and metallic gold (i.e., gold in the zero oxidation state) and metallic silver (i.e., silver in the zero oxidation state). In another embodiment of this invention, the catalyst composition comprises alpha-chromium oxide (i.e., □-Cr₂O₃) and metallic gold (i.e., gold in the zero oxidation state) and palladium. In another embodiment of this invention, the catalyst composition comprises alpha-chromium oxide (i.e., □-Cr₂O₃) and metallic silver (i.e., silver in the zero oxidation state) and palladium. In another embodiment of this invention, the catalyst composition comprises alpha-chromium oxide (i.e., □-Cr₂O₃), metallic gold (i.e., gold in the zero oxidation state), metallic silver (i.e., silver in the zero oxidation state), and palladium. Of note are embodiments wherein at least 50 weight % of the chromium component is present as alpha-chromium oxide. Also of note are embodiments wherein the gold component consists essentially of metallic gold having an average particle size of from about 1 nanometer to about 500 nanometers. In certain embodiments of this invention, particles of metallic gold and at least one of palladium and metallic silver are dispersed in a matrix comprising chromium oxide. In certain embodiments of this invention, particles of metallic silver and palladium are dispersed in a matrix comprising chromium oxide.

In certain embodiments of this invention, particles of metallic gold and at least one of palladium and metallic silver are supported on a chromium oxide support. In some embodiments, particles of metallic silver and palladium are supported on a chromium oxide support.

The catalyst compositions of this invention may further comprise fluorine as an essential constituent element.

The amount of modifier metals relative to the total amount of chromium and modifier metals in the catalyst compositions used for the chlorofluorination reaction is preferably from about 0.5 atom % to about 5 atom %.

Further information on catalyst compositions comprising chromium, oxygen, and at least two modifier metals selected from the group consisting of gold, silver, and palladium as essential constituent elements useful for this invention (including embodiments further comprising fluorine) is provided in Invention Category A above and in U.S. Patent Applications 60/903,218 [FL 1356 US PRV] filed Feb. 23, 2007, and 60/927,846 [FL 1356 US PRV1] filed May 4, 2007, hereby incorporated herein by reference in their entirety.

The modifier metal-containing chromium oxide catalysts used in the present invention can be formed into various shapes such as pellets, granules, and extrudates for use in packing reactors. They can also be used in powder forms.

The catalyst compositions used in step (a) of this invention may further comprise one or more additives in the form of metal compounds. Such additives may alter the selectivity and/or activity of the modifier metal-containing chromium oxide catalyst compositions or the fluorinated modifier metal-containing chromium oxide catalyst compositions. Suitable additives can be selected from the group consisting of the fluorides, oxides, and oxyfluoride compounds of Mg, Ca, Sc, Y, La, Ti, Zr, W, Mn, Re, Fe, Ru, Co, Rh, Ir, Ni, Pt, Ce, and Zn.

The total content of the additive(s) in the catalyst compositions used in step (a) of the present invention may be from about 0.05 weight % to about 10 weight % based on the total metal content of the catalyst compositions. The additives may be incorporated into the catalyst compositions by standard procedures such as by impregnation or during co-precipitation of the modifier metal and chromium salts.

The catalyst compositions used in step (a) of the present invention can be treated with a fluorinating agent to form catalyst compositions comprising chromium, oxygen, modifier metals and fluorine as essential elements. Typically, prior to use as catalysts, the catalyst compositions are pre-treated with a fluorinating agent. Typically this fluorinating agent is HF though other materials may be used such as sulfur tetrafluoride, carbonyl fluoride, and fluorinated hydrocarbon compounds such as trichlorofluoromethane, dichlorodifluoromethane, chlorodifluoromethane, trifluoromethane, and 1,1,2-trichlorotrifluoroethane. This pretreatment can be accomplished, for example, by placing the catalyst composition in a suitable container which can also be the reactor to be used to perform the process in the present invention, and thereafter, passing HF over the calcined catalyst composition so as to partially saturate the catalyst composition with HF. This can be conveniently carried out by passing HF over the catalyst composition for a period of time, for example, about 0.1 to about 10 hours at a temperature of, for example, about 200° C. to about 450° C. Nevertheless, this pre-treatment is not essential.

Compounds that are produced by the chlorofluorination process in step (a) include the halopropanes CF₃CCl₂CClF₂ (CFC-215aa) and CF₃CClFCCl₂F (CFC-215bb).

Halopropane by-products that have a higher degree of fluorination than CFC-215aa and CFC-215bb that may be produced in step (a) include CF₃CCl₂CF₃ (CFC-216aa), CF₃CClFCClF₂ (CFC-216ba), CF₃CF₂CCl₂F (CFC-216cb), CF₃CClFCF₃ (CFC-217ba), and CF₃CHClCF₃ (HCFC-226da).

Halopropane by-products that may be formed in step (a) which have lower degrees of fluorination than CFC-215aa and CFC-215bb include CF₃CCl₂CCl₂F (HCFC-214ab) and CF₃CCl₂CCl₃ (HCFC-213ab).

Halopropene by-products that may be formed in step (a) include CF₃CCl═CF₂ (CFC-1215xc), E- and Z-CF₃CCl═CClF (CFC-1214xb), and CF₃CCl═CCl₂ (CFC-1213xa).

By proper selection of the operating variables, such as temperature, pressure, contact time and reactant ratios, conversion to compounds having a higher degree of fluorination than trichloropentafluoropropanes can be minimized if needed.

Prior to step (b), CF₃CCl₂CClF₂ (CFC-215aa) and CF₃CClFCCl₂F (CFC-215bb) (and optionally HF) from the effluent from step (a) are typically separated from lower boiling components of the effluent (which typically comprise HCl, Cl₂, HF and overfluorinated products such as C₃ClF₇ and C₃Cl₂F₆ isomers) and the underfluorinated components of the effluent (which typically comprise C₃Cl₄F₄ isomers, CFC-213ab and/or underhalogenated components such as C₃ClF₅ and C₃Cl₂F₄ isomers and CFC-1213xa). Underfluorinated and underhalogenated components (e.g., CFC-214ab, CFC-1212xb, and CFC-1213xa) may be returned to step (a).

In one embodiment of the present invention, the CFC-216aa, and CFC-216ba produced in step (a) are further reacted with hydrogen (H₂), optionally in the presence of HF, to produce 1,1,1,3,3,3-hexafluoropropane (HFC-236fa), and at least one of 1,1,1,2,3,3-hexafluoropropane (HFC-236ea), and hexafluoropropene (HFP) as disclosed in U.S. Patent Application 60/927,847 [FL 1367 US PRV], filed May 4, 2007 and hereby incorporated herein by reference.

In another embodiment of the invention, the reactor effluent from step (a) may be delivered to a first distillation column in which HCl and any HCl azeotropes are removed from the top of column while the higher boiling components are removed at the bottom of the column. The products recovered at the bottom of the first distillation column are then delivered to a second distillation column in which HF, Cl₂, CF₃CCl₂CF₃ (CFC-216aa), CF₃CClFCClF₂ (CFC-216ba), CF₃CF₂CCl₂F (CFC-216cb), CF₃CClFCF₃ (CFC-217ba), and CF₃CHClCF₃ (HCFC-226da) and their HF azeotropes are recovered at the top of the column and CFC-215aa and CFC-215bb, and any remaining HF and the higher boiling components are removed from the bottom of the column. The products recovered from the bottom of the second distillation column may then be delivered to a further distillation column to separate the underfluorinated by-products and intermediates to isolate CFC-215aa and CFC-215bb.

Optionally, after distillation and separation of HCl from the reactor effluent of step (a), the resulting mixture of HF and halopropanes and halopropenes may be delivered to a decanter controlled at a suitable temperature to permit separation of a liquid HF-rich phase and a liquid organic-rich phase. The organic-rich phase may then be processed to isolate the CFC-215aa and CFC-215bb. The HF-rich phase may then be recycled to the reactor of step (a), optionally after removal of any organic components. The decantation step may be used at other points in the CFC-215aa/CFC-215bb separation scheme where HF is present.

In step (b) of the process of this invention, CF₃CCl₂CClF₂ (CFC-215aa) and CF₃CClFCCl₂F (CFC-215bb) produced in step (a) are reacted with hydrogen (H₂) in a second reaction zone.

In one embodiment of step (b), a mixture comprising CFC-215aa and CFC-215bb is delivered in the vapor phase, along with hydrogen (H₂), to a reactor containing a hydrogenation catalyst. Hydrogenation catalysts suitable for use in this embodiment include catalysts comprising at least one metal selected from the group consisting of iron, ruthenium, rhodium, iridium, palladium, and platinum. Said catalytic metal component is typically supported on a carrier such as carbon or graphite.

Of note are carbon supported catalysts in which the carbon support has been washed with acid and has an ash content below about 0.1% by weight. Hydrogenation catalysts supported on low ash carbon are described in U.S. Pat. No. 5,136,113, the teachings of which are incorporated herein by reference.

Of particular note are catalysts containing palladium supported on carbon. The hydrogenation of CFC-215aa and CFC-215bb to produce HFC-245fa and HFC-245eb is disclosed in International Publication No. WO 2005/037743 A1, which is incorporated herein by reference.

The supported metal catalysts may be prepared by conventional methods known in the art such as by impregnation of the carrier with a soluble salt of the catalytic metal (e.g., palladium chloride or rhodium nitrate) as described by Satterfield on page 95 of Heterogenous Catalysis in Industrial Practice, 2^{nd} edition (McGraw-Hill, New York, 1991). The concentration of the catalytic metal(s) on the support is typically in the range of about 0.1% by weight of the catalyst to about 5% by weight.

The relative amount of hydrogen contacted with CFC-215aa and CFC-215bb (i.e., trichloropentafluoropropanes, C₃Cl₃F₅ isomers) in the presence of a hydrogenation catalyst is typically from about 0.5 mole of H₂ per mole of trichloropentafluoropropane isomer to about 10 moles of H₂ per mole of trichloropentafluoropropane isomer, preferably from about 3 moles of H₂ per mole of trichloropentafluoropropane isomer to about 8 moles of H₂ per mole of trichloropentafluoropropane isomer.

Suitable temperatures for the catalytic hydrogenation are typically in the range of from about 100° C. to about 350° C., preferably from about 125° C. to about 300° C. Temperatures above about 350° C. tend to result in defluorination side reactions; temperatures below about 125° C. will result in incomplete substitution of Cl for H in the C₃Cl₃F₅ starting materials. The reactions are typically conducted at atmospheric pressure or superatmospheric pressure.

The effluent from the step (b) reaction zone typically includes HCl, unreacted hydrogen, CF₃CH₂CHF₂ (HFC-245fa), CF₃CHFCH₂F (HFC-245eb), lower boiling by-products (typically including CF₃CH═CF₂ (HFC-1225zc), E- and Z-CF₃CH═CHF (HFC-1234ze), CF₃CF═CH₂ (HFC-1234yf), CF₃CH₂CF₃ (HFC-236fa), CF₃CHFCH₃ (HFC-254eb), and/or CF₃CH₂CH₃ (HFC-263fb)) and higher boiling by-products and intermediates (typically including CF₃CH₂CH₂Cl (HCFC-253fb), CF₃CHFCH₂Cl (HCFC-244eb), CF₃CClFCH₂F (HCFC-235bb), CF₃CHClCHF₂ (HCFC-235da), CF₃CHClCClF₂ (HCFC-225da), and/or CF₃CClFCHClF (HCFC-225ba diasteromers)) as well as any HF carried over from step (a) or step (b).

In one embodiment of this invention, HFC-245fa and HFC-245eb produced in step (b) are recovered as disclosed in U.S. Patent Application 60/927,848 [FL-1365 US PRV] filed May 4, 2007 and hereby incorporated herein by reference.

In step (c) of the process, HFC-245fa and HFC-245eb produced in step (b) are dehydrofluorinated.

In one embodiment of step (c), a mixture comprising HFC-245fa and HFC-245eb, and optionally an inert gas, is delivered in the vapor phase to a reaction zone containing a dehydrofluorination catalyst as described in U.S. Pat. No. 6,369,284; the teachings of this disclosure are incorporated herein by reference. Dehydrofluorination catalysts suitable for use in this embodiment include (1) at least one compound selected from the oxides, fluorides and oxyfluorides of magnesium, zinc and mixtures of magnesium and zinc, (2) lanthanum oxide, (3) fluorided lanthanum oxide, (4) activated carbon, and (5) three-dimensional matrix carbonaceous materials.

The catalytic dehydrofluorination of CF₃CH₂CHF₂ and CF₃CHFCH₂F is suitably conducted at a temperature in the range of from about 200° C. to about 500° C., and preferably from about 350° C. to about 450° C. The contact time is typically from about 1 to about 450 seconds, preferably from about 10 to about 120 seconds.

The reaction pressure can be subatmospheric, atmospheric or superatmospheric. Generally, near atmospheric pressures are preferred. However, the dehydrofluorination of CF₃CH₂CHF₂ and CF₃CHFCH₂F can be beneficially run under reduced pressure (i.e., pressures less than one atmosphere).

The catalytic dehydrofluorination can optionally be carried out in the presence of an inert gas such as nitrogen, helium or argon. The addition of an inert gas can be used to increase the extent of dehydrofluorination. Of note are processes where the mole ratio of inert gas to CF₃CH₂CHF₂ and/or CF₃CHFCH₂F is from about 5:1 to 1:1. Nitrogen is the preferred inert gas.

The products from the step (c) reaction zone typically include HF, E- and Z-forms of CF₃CH═CHF (HFC-1234ze), CF₃CF═CH₂ (HFC-1234ye), CF₃CH₂CHF₂, CF₃CHFCH₂F and small amounts of other products. Unconverted CF₃CH₂CHF₂ and CF₃CHFCH₂F are recycled back to the dehydrofluorination reactor to produce additional quantities of CF₃CH═CHF and CF₃CF═CH₂.

In another embodiment of step (c), the HFC-245fa and HFC-245eb are subjected to dehydrofluorination at an elevated temperature in the absence of a catalyst as disclosed in U.S. Patent Application Publication No. 2006/0094911 which is incorporated herein by reference. The reactor can be fabricated from nickel, iron, titanium, or their alloys, as described in U.S. Pat. No. 6,540,933; the teachings of this disclosure are incorporated herein by reference.

The temperature of the reaction in this embodiment can be between about 350° C. and about 900° C., and is preferably at least about 450° C.

In yet another embodiment of step (c), the HFC-245fa and HFC-245eb are dehydrofluorinated by reaction with caustic (e.g., KOH). The vapor-phase dehydrofluorination reaction of CF₃CHFCHF₂ with caustic to produce both CF₃CH═CF₂ and CF₃CF═CHF is disclosed by Sianesi, et. al., Ann. Chim., 55, 850-861 (1965) and the liquid-phase dehydrofluorination of CF₃CH₂CHF₂ and CF₃CHFCH₂F in di-n-butyl ether, by reaction with caustic, to produce CF₃CH═CHF and CF₃CF═CH₂ is disclosed by Knunyants, et. al., Izv. Akad. Nauk. SSSR, 1960, pp. 1412-1418, Chem. Abstracts 55, 349f the teachings of which are incorporated herein by reference.

In step (d) of the process of this invention, the CF₃CH═CHF, CF₃CF═CH₂, or both CF₃CH═CHF and CF₃CF═CH₂, produced in (c) are recovered individually and/or as one or more mixtures of CF₃CH═CHF and CF₃CF═CH₂ by well known procedures, such as distillation.

CF₃CH═CHF, CF₃CF═CH₂, or mixtures thereof, may be used as refrigerants, foam expansion agents or chemical intermediates. Of note is a foam expansion agent comprising a mixture of CF₃CH═CHF and CF₃CF═CH₂ produced in accordance with this invention.

Embodiments of this invention include, but are not limited to:

Embodiment C1. A process for the manufacture of at least one compound selected from the group consisting of 1,3,3,3-tetrafluoropropene and 2,3,3,3-tetrafluoropropene, comprising (a) reacting hydrogen fluoride, chlorine, and at least one halopropene of the formula CX₃CCl═CClX, wherein each X is independently selected from the group consisting of F and Cl, to produce a product comprising CF₃CCl₂CClF₂ and CF₃CClFCCl₂F, wherein said CF₃CCl₂CClF₂ and CF₃CClFCCl₂F are produced in the presence of a catalyst composition comprising chromium, oxygen, and at least two modifier metals selected from the group consisting of gold, silver, and palladium as essential constituent elements, wherein the total amount of modifier metals in said catalyst composition is from about 0.05 atom % to about 10 atom % based on the total amount of chromium and modifier metals in the catalyst composition; (b) reacting CF₃CCl₂CClF₂ and CF₃CClFCCl₂F produced in (a) with hydrogen to produce a product comprising CF₃CH₂CHF₂ and CF₃CHFCH₂F; (c) dehydrofluorinating CF₃CH₂CHF₂ and CF₃CHFCH₂F produced in (b) to produce a product comprising CF₃CH═CHF and CF₃CF═CH₂; and (d) recovering at least one compound selected from the group consisting of CF₃CH═CHF and CF₃CF═CH₂ from the product produced in (c).

Embodiment C2. The process of Embodiment C1 wherein the halopropene reactant is contacted with Cl₂ and HF in a pre-reactor.

Embodiment C3. The process of Embodiment C1 wherein the halopropene reactant is contacted with HF in a pre-reactor.

Embodiment C4. The process of Embodiment C1 wherein the reaction of (b) is conducted in a reaction zone containing a hydrogenation catalyst at a temperature of from about 100° C. to about 350° C.

Embodiment C5. The process of Embodiment C1 wherein the reaction of (c) is conducted in the absence of a catalyst at a temperature of from about 350° C. to about 900° C.

Embodiment C6. The process of Embodiment C1 wherein the reaction of (c) is conducted in a reaction zone containing a dehydrofluorination catalyst at a temperature of from about 200° C. to about 500° C.

Embodiment C7. The process of Embodiment C1 wherein the amount of modifier metals relative to the total amount of chromium and modifier metals in the catalyst composition is from about 0.5 atom % to about 5 atom %.

Embodiment C8. The process of Embodiment C1 wherein the catalyst composition further comprises fluorine as an essential constituent element.

Embodiment C9. The process of Embodiment C1 wherein the catalyst composition comprises alpha-chromium oxide, metallic gold and metallic silver.

Embodiment C10. The process of Embodiment C1 wherein the catalyst composition comprises alpha-chromium oxide, metallic gold and palladium.

Embodiment C11. The process of Embodiment C1 wherein the catalyst composition comprises alpha-chromium oxide, metallic silver and palladium.

Embodiment C12. The process of Embodiment C1 wherein the catalyst composition comprises alpha-chromium oxide, metallic gold, metallic silver and palladium.

Embodiment C13. The process of Embodiment C1 wherein the catalyst composition comprises silver and gold and the mole ratio of silver to gold is from about 10:1 to about 1:10.

Embodiment C14. The process of Embodiment C1 wherein the catalyst composition comprises silver and palladium and the mole ratio of silver to palladium is from about 10:1 to about 1:10.

Embodiment C15. The process of Embodiment C1 wherein the catalyst composition comprises palladium and gold and the mole ratio of palladium to gold is from about 10:1 to about 1:10.

### Examples

Reference is made to Examples A6-A10 in Invention Category A above for the chlorofluorination of CFC-1213xa.

Examination of the data shown in Table A2 above show that the amount of CFC-215aa and CFC-215bb can be maximized relative to CFC-216aa and CFC-216ba by controlling the operational variables by using the catalysts of this invention. The CFC-215aa and CFC-215bb produced above may be hydrogenated to produce HFC-245fa and HFC-245eb, respectively, in a manner analogous to the teachings of International Publication No. WO 2005/037743 A1. The HFC-245fa and HFC-245eb may be dehydrofluorinated to HFC-1234ze and HFC-1234yf, respectively, in accordance with the teachings described in U.S. Pat. No. 6,369,284. The HFC-1234ze and HFC-1234yf may be recovered individually or as mixtures of HFC-1234ze and HFC-1234yf by procedures known to the art.

### D.

Invention Category D of this application provides a process for the preparation of CF₃CH₂CF₃ (HFC-236fa) and CF₃CHFCHF₂ (HFC-236ea). This invention also provides a process for the preparation of HFC-236fa, HFC-236ea and CF₃CF═CF₂ (HFP).

In step (a) of the process of this invention, one or more halopropene starting materials CX₃CCl═CClX, wherein each X is independently selected from the group consisting of F and Cl, are reacted with chlorine (Cl₂) and hydrogen fluoride (HF) to produce a product mixture comprising CF₃CCl₂CF₃ (CFC-216aa) and CF₃CClFCClF₂ (CFC-216ba). Accordingly, this invention also provides a process for the preparation of mixtures of CF₃CCl₂CF₃ (CFC-216aa) and CF₃CClFCClF₂ (CFC-216ba) from readily available starting materials.

Suitable starting materials for the process of this invention include E- and Z-CF₃CCl═CClF (CFC-1214xb), CF₃CCl═CCl₂ (CFC-1213xa), CClF₂CCl═CCl₂ (CFC-1212xa), CCl₂FCCl═CCl₂ (CFC-1211xa), and CCl₃CCl═CCl₂ (hexachloropropene, HCP), or mixtures thereof.

Due to their availability, CF₃CCl═CCl₂ (CFC-1213xa) and CCl₃CCl═CCl₂ (hexachloropropene, HCP) are the preferred halopropene starting materials for the process of the invention.

Preferably, the reaction of HF and Cl₂ with the halopropenes CX₃CCl═CClX is carried out in the vapor phase in a heated tubular reactor. A number of reactor configurations are possible, including vertical and horizontal orientation of the reactor and different modes of contacting the halopropene starting material(s) with HF and chlorine. Preferably the HF and chlorine are substantially anhydrous.

In one embodiment of step (a), the halopropene starting material(s) are fed to the reactor containing the chlorofluorination catalyst. The halopropene starting material(s) may be initially vaporized and fed to the reaction zone as gas(es).

In another embodiment of step (a), the halopropene starting material(s) may be contacted with HF in a pre-reactor (i.e. prior to contacting the chlorofluorination catalyst). The pre-reactor may be empty (i.e., unpacked), but is preferably filled with a suitable packing such as Monel™ or Hastelloy™ nickel alloy turnings or wool, or other material inert to HCl and HF, that allows for efficient mixing of CX₃CCl═CClX and HF vapor.

If the halopropene starting material(s) are fed to the pre-reactor as liquid(s), it is preferable for the pre-reactor to be oriented vertically with CX₃CCl═CClX entering the top of the reactor and pre-heated HF vapor introduced at the bottom of the reactor.

Suitable temperatures for the pre-reactor are within the range of from about 80° C. to about 250° C., preferably from about 100° C. to about 200° C. Under these conditions, for example, hexachloropropene is converted to a mixture containing predominantly CFC-1213xa. The feed rate of the starting material is determined by the length and diameter of the reactor, reactor temperature, and the degree of fluorination desired in the pre-reactor. Slower feed rates at a given temperature will increase contact time and tend to increase the amount of conversion of the starting material and increase the degree of fluorination of the products.

The term “degree of fluorination” means the extent to which fluorine atoms replace chlorine substituents in the CX₃CCl═CClX starting materials. For example, CF₃CCl═CClF represents a higher degree of fluorination than CClF₂CCl═CCl₂ and CF₃CCl₂CF₃ represents a higher degree of fluorination than CClF₂CCl₂CF₃.

The molar ratio of HF fed to the pre-reactor, or otherwise to the reaction zone of step (a), to halopropene starting material fed in step (a) is typically from about stoichiometric to about 50:1. The stoichiometric ratio depends on the average degree of fluorination of the halopropene starting material(s) and is typically based on formation of C₃Cl₂F₆ isomers. For example, if the halopropene is HCP, the stoichiometric ratio of HF to HCP is 6:1; if the halopropene is CFC-1213xa, the stoichiometric ratio of HF to CFC-1213xa is 3:1. Preferably, the molar ratio of HF to halopropene starting material is from about twice the stoichiometric ratio (based on formation of C₃Cl₂F₆ isomers) to about 30:1. Higher ratios of HF to halopropene are not particularly beneficial. Lower ratios result in reduced yields of C₃Cl₂F₆ isomers.

If the halopropene starting materials are contacted with HF in a pre-reactor, the effluent from the pre-reactor is then contacted with chlorine in the reaction zone of step (a).

In another embodiment of the invention, the halopropene starting material(s) may be contacted with Cl₂ and HF in a pre-reactor (i.e. prior to contacting the chlorofluorination catalyst). The pre-reactor may be empty (i.e., unpacked) but is preferably filled with a suitable packing such as Monel™ or Hastelloy™ nickel alloy turnings or wool, activated carbon, or other material inert to HCl, HF, and Cl₂ that allows for efficient mixing of CX₃CCl═CClX, HF, and Cl₂.

Typically, at least a portion of the halopropene starting material(s) react(s) with Cl₂ and HF in the pre-reactor by addition of Cl₂ to the olefinic bond to give a saturated halopropane as well as by substitution of at least a portion of the Cl substituents in the halopropropane and/or halopropene by F. Suitable temperatures for the pre-reactor in this embodiment of the invention are within the range of from about 80° C. to about 250° C., preferably from about 100° C. to about 200° C. Higher temperatures result in greater conversion of the halopropene(s) entering the reactor to saturated products and greater degrees of halogenation and fluorination in the pre-reactor products.

The term “degree of halogenation” means the extent to which hydrogen substituents in a halocarbon have been replaced by halogen and carbon-carbon double bonds have been saturated with halogen. For example, CF₃CCl₂CClF₂ has a higher degree of halogenation than CF₃CCl═CCl₂. Also, CF₃CCl₂CClF₂ has a higher degree of halogenation than CF₃CHClCClF₂.

The molar ratio of Cl₂ to halopropene starting material(s) is typically from about 1:1 to about 10:1, and is preferably from about 1:1 to about 5:1. Feeding Cl₂ at less than a 1:1 ratio will result in the presence of relatively large amounts of unsaturated materials and hydrogen-containing side products in the reactor effluent.

In a preferred embodiment of step (a), the halopropene starting materials are vaporized, preferably in the presence of HF, and contacted with HF and Cl₂ in a pre-reactor and then contacted with the chlorofluorination catalyst. If the preferred amounts of HF and Cl₂ are fed to the pre-reactor, additional HF and Cl₂ are not required in the reaction zone.

Suitable temperatures in the reaction zone(s) of step (a) are within the range of from about 200° C. to about 400° C., preferably from about 250° C. to about 350° C., depending on the desired conversion of the starting material and the activity of the catalyst. Reactor temperatures greater than about 350° C. may result in products having a degree of fluorination greater than five. In other words, at higher temperatures, substantial amounts of chloropropanes containing six or more fluorine substituents (e.g., CF₃CCl₂CF₃ or CF₃CClFCClF₂) may be formed. Reactor temperatures below about 240° C. may result in a substantial yield of products with a degree of fluorination less than five (i.e., underfluorinates).

Suitable reactor pressures for vapor phase embodiments of this invention may be in the range of from about 1 to about 30 atmospheres. Reactor pressures of about 5 atmospheres to about 20 atmospheres may be advantageously employed to facilitate separation of HCl from other reaction products.

The chlorofluorination catalysts used in step (a) of this invention comprise chromium, oxygen and modifier metals (e.g., modifier metal-containing chromium oxide) and contain from about 0.05 atom % to about 10 atom % modifier metals based on the total amount of modifier metals and chromium in the catalyst composition. Of note are compositions comprising silver and gold wherein the mole ratio of silver to gold is from about 10:1 to about 1:10. Also of note are compositions comprising silver and palladium wherein the mole ratio of silver to palladium is from about 10:1 to about 1:10. Also of note are compositions comprising palladium and gold wherein the mole ratio of palladium to gold is from about 10:1 to about 1:10.

In one embodiment of this invention, the catalyst composition comprises alpha-chromium oxide (i.e., □-Cr₂O₃) and metallic gold (i.e., gold in the zero oxidation state) and metallic silver (i.e., silver in the zero oxidation state). In another embodiment of this invention, the catalyst composition comprises alpha-chromium oxide (i.e., □-Cr₂O₃) and metallic gold (i.e., gold in the zero oxidation state) and palladium. In another embodiment of this invention, the catalyst composition comprises alpha-chromium oxide (i.e., □-Cr₂O₃) and metallic silver (i.e., silver in the zero oxidation state) and palladium. In another embodiment of this invention, the catalyst composition comprises alpha-chromium oxide (i.e., □-Cr₂O₃), metallic gold (i.e., gold in the zero oxidation state), metallic silver (i.e., silver in the zero oxidation state), and palladium. Of note are embodiments wherein at least 50 weight % of the chromium component is present as alpha-chromium oxide. Also of note are embodiments wherein the gold component consists essentially of metallic gold having an average particle size of from about 1 nanometer to about 500 nanometers. In certain embodiments of this invention, particles of metallic gold and at least one of palladium and metallic silver are dispersed in a matrix comprising chromium oxide. In certain embodiments of this invention, particles of metallic silver and palladium are dispersed in a matrix comprising chromium oxide.

In certain embodiments of this invention, particles of metallic gold and at least one of palladium and metallic silver are supported on a chromium oxide support. In some embodiments, particles of metallic silver and palladium are supported on a chromium oxide support.

The catalyst compositions of this invention may further comprise fluorine as an essential constituent element.

The amount of modifier metals relative to the total amount of chromium and modifier metals in the catalyst compositions used for the chlorofluorination reaction is preferably from about 0.5 atom % to about 5 atom %.

Further information on catalyst compositions comprising chromium, oxygen, and at least two modifier metals selected from the group consisting of gold, silver, and palladium as essential constituent elements useful for this invention (including embodiments further comprising fluorine) is provided in Invention Category A above and in U.S. Patent Applications 60/903,218 [FL 1356 US PRV] filed Feb. 23, 2007, and 60/927,846 [FL 1356 US PRV1] filed May 4, 2007, hereby incorporated herein by reference in their entirety.

The modifier metal-containing chromium oxide catalysts used in the present invention can be formed into various shapes such as pellets, granules, and extrudates for use in packing reactors. They can also be used in powder forms.

The catalyst compositions used in step (a) of this invention may further comprise one or more additives in the form of metal compounds. Such additives may alter the selectivity and/or activity of the modifier metal-containing chromium oxide catalyst compositions or the fluorinated modifier metal-containing chromium oxide catalyst compositions. Suitable additives can be selected from the group consisting of the fluorides, oxides, and oxyfluoride compounds of Mg, Ca, Sc, Y, La, Ti, Zr, W, Mn, Re, Fe, Ru, Co, Rh, Ir, Ni, Pt, Ce, and Zn.

The total content of the additive(s) in the catalyst compositions used in step (a) of the present invention may be from about 0.05 weight % to about 10 weight % based on the total metal content of the catalyst compositions. The additives may be incorporated into the catalyst compositions by standard procedures such as by impregnation or during co-precipitation of the modifier metal and chromium salts.

The catalyst compositions used in step (a) of the present invention can be treated with a fluorinating agent to form catalyst compositions comprising chromium, oxygen, modifier metals and fluorine as essential elements. Typically, prior to use as catalysts, the catalyst compositions are pre-treated with a fluorinating agent. Typically this fluorinating agent is HF though other materials may be used such as sulfur tetrafluoride, carbonyl fluoride, and fluorinated hydrocarbon compounds such as trichlorofluoromethane, dichlorodifluoromethane, chlorodifluoromethane, trifluoromethane, and 1,1,2-trichlorotrifluoroethane. This pretreatment can be accomplished, for example, by placing the catalyst composition in a suitable container which can also be the reactor to be used to perform the process in the present invention, and thereafter, passing HF over the calcined catalyst composition so as to partially saturate the catalyst composition with HF. This can be conveniently carried out by passing HF over the catalyst composition for a period of time, for example, about 0.1 to about 10 hours at a temperature of, for example, about 200° C. to about 450° C. Nevertheless, this pre-treatment is not essential.

Compounds that are produced in the chlorofluorination process step (a) include the halopropanes CF₃CCl₂CF₃ (CFC-216aa) and CF₃CClFCClF₂ (CFC-216ba).

Halopropane by-products that have a higher degree of fluorination than CFC-216aa and CFC-216ba that may be produced in step (a) include CF₃CClFCF₃ (CFC-217ba) and CF₃CF₂CF₃ (FC-218).

Halopropane and halopropene by-products that may be formed in step (a) which have lower degrees of fluorination and/or halogenation than CFC-216aa and CFC-216ba include CF₃CCl₂CClF₂ (CFC-215aa), CF₃CClFCCl₂F (CFC-215bb), CF₃CCl₂CCl₂F (CFC-214ab), and CF₃CCl═CF₂ (CFC-1215xc).

Prior to step (b), the CF₃CCl₂CF₃ and CF₃CClFCClF₂, (and optionally HF) in the effluent from the reaction zone in step (a), are typically separated from the low boiling components of the effluent (which typically comprise HCl, Cl₂, HF, and overfluorinated products such as CF₃CClFCF₃) and the underfluorinated components (which typically comprise C₃Cl₃F₅ (e.g., CFC-215aa and CFC-215bb) isomers, C₃Cl₄F₄ isomers, and/or underhalogenated components such as C₃Cl₂F₄ isomers and CF₃CCl═CCl₂). The higher boiling components may be returned to step (a).

In one embodiment of this invention, the underfluorinated components CFC-215aa and CFC-215bb are converted to CF₃CH₂CHF₂ (HFC-245fa) and CF₃CHFCH₂F (HFC-245eb) as disclosed in U.S. Patent Application 60/927,848 [FL-1365 US PRV] filed May 4, 2007 and hereby incorporated herein by reference.

In another embodiment of this invention, the reactor effluent from step (a) is delivered to a first distillation column in which HCl and any HCl azeotropes are removed from the top of the column while the higher boiling components are removed from the bottom of the column. The products recovered from the bottom of the first distillation column are then delivered to a second distillation column in which HF, Cl₂, and any CFC-217ba are recovered at the top of the second distillation column and remaining HF and organic products, comprising CF₃CCl₂CF₃ and CF₃CClFCClF₂, are recovered at the bottom of the second distillation column. The products recovered from the bottom of the second distillation column may be delivered to further distillation columns or may be delivered to a decanter controlled at a suitable temperature to permit separation of an organic-rich phase and an HF-rich phase. The HF-rich phase may be distilled to recover HF that is then recycled to step (a). The organic-rich phase may then be delivered to step (b).

In step (b) of the process of this invention, CF₃CCl₂CF₃ and CF₃CClFCClF₂ are contacted with hydrogen (H₂), optionally in the presence of HF, in a second reaction zone. The CF₃CCl₂CF₃ and CF₃CClFCClF₂ may be fed to the reaction zone at least in part as their azeotropes with HF.

In one embodiment of step (b), a mixture comprising CF₃CCl₂CF₃ and CF₃CClFCClF₂, and optionally containing HF, is delivered in the vapor phase, along with hydrogen, to a reactor fabricated from nickel, iron, titanium, or their alloys, as described in U.S. Pat. No. 6,540,933; the teachings of this disclosure are incorporated herein by reference.

The temperature of the reaction in this embodiment of step (b) can be between about 350° C. to about 800° C., and is preferably at least about 450° C. Of note are processes wherein the reaction of (b) is conducted in a reaction zone at a temperature of from about 350° C. to about 600° C. which is unpacked or packed with a nickel alloy.

The molar ratio of hydrogen to the CFC-216aa/CFC-216ba mixture fed to the reaction zone should be in the range of about 0.1 mole H₂ per mole of CFC-216 isomer to about 60 moles of H₂ per mole of CFC-216 isomer, more preferably from about 0.4 to 10 moles of H₂ per mole of CFC-216 isomer.

Alternatively, the contacting of hydrogen with the mixture of CFC-216aa and CFC-216ba, and optionally HF, is carried out in the presence of a hydrogenation catalyst. In this embodiment of step (b), said mixture is delivered in the vapor phase, along with hydrogen, to the reaction zone containing a hydrogenation catalyst according to the teachings disclosed in U.S. Patent Application No. 60/706,161 filed on Aug. 5, 2005 and incorporated herein by reference. Hydrogenation catalysts suitable for use in this embodiment include catalysts comprising at least one metal selected from the group consisting of iron, ruthenium, rhodium, iridium, palladium, and platinum. Said catalytic metal component is typically supported on a carrier such as carbon or graphite or a metal oxide, fluorinated metal oxide, or metal fluoride where the carrier metal is selected from the group consisting of magnesium, aluminum, titanium, vanadium, chromium, iron, and lanthanum. Preferred catalysts for the hydrogenolysis include palladium supported on fluorided alumina or carbon. The hydrogenolysis of saturated acyclic halofluorocarbons containing 3 or 4 carbon atoms using palladium supported on carbon is disclosed in U.S. Pat. No. 5,523,501, the teachings of which are incorporated herein by reference.

The supported metal catalysts may be prepared by conventional methods known in the art such as by impregnation of the carrier with a soluble salt of the catalytic metal (e.g., palladium chloride or rhodium nitrate) as described by Satterfield on page 95 of Heterogenous Catalysis in Industrial Practice, 2^{nd} edition (McGraw-Hill, New York, 1991). The concentration of the catalytic metal(s) on the support is typically in the range of about 0.1% by weight of the catalyst to about 5% by weight.

Suitable temperatures for the reaction zone containing said hydrogenation catalyst are in the range of from about 100° C. to about 350° C., preferably from about 125° C. to about 300° C. Higher temperatures typically result in greater conversion of CFC-216aa and CFC-216ba with fewer partially chlorinated intermediates such as C₃HClF₆ isomers.

The amount of hydrogen (H₂) fed to the reaction zone containing said hydrogenation catalyst is typically from about 1 mole of H₂ per mole of dichlorohexafluoropropane to about 20 moles of H₂ per mole of dichlorohexafluoropropane, preferably from about 2 moles of H₂ per mole of dichlorohexafluoropropane to about 10 moles of H₂ per mole of dichlorohexafluoropropane.

The pressure used in the step (b) reaction zone is not critical and may be in the range of from about 1 to 30 atmospheres. A pressure of about 20 atmospheres may be advantageously employed to facilitate separation of HCl from other reaction products.

The effluent from the step (b) reaction zone typically includes HCl, unreacted hydrogen, CF₃CF═CF₂ (HFP), CF₃CH₂CF₃ (HFC-236fa) and CF₃CHFCHF₂ (HFC-236ea), as well as any HF carried over from step (a) or step (b). In addition, small amounts of CF₃CF₂CH₂F (HFC-236cb), CF₃CCl═CF₂ (CFC-1215xc), and partially chlorinated by-products such as C₃HClF₆ isomers including CF₃CHClCF₃ (HCFC-226da), CF₃CClFCHF₂ (HCFC-226ba), CF₃CHFCClF₂ (HCFC-226ea), may be formed.

In step (c), the desired products are recovered. The reactor effluent from step (b) may be delivered to a separation unit to recover CF₃CH₂CF₃ and at least one of CF₃CHFCHF₂ and CF₃CF═CF₂. Typically, CF₃CF═CF₂, if present, is recovered separately from CF₃CH₂CF₃ and any CF₃CHFCHF₂. Typically, CF₃CHFCHF₂, if present, is recovered as a mixture with CF₃CH₂CF₃. Separation can be accomplished by well-known procedures such as by distillation.

In one embodiment of this invention, CF₃CH₂CF₃ and CF₃CHFCHF₂ from step (b) are dehydrofluorinated to produce CF₃CH═CF₂ and CF₃CF═CHF as disclosed in U.S. Patent Application 60/927,839 [FL-1364 US PRV] filed May 4, 2007 and hereby incorporated herein by reference.

The partially chlorinated by-products, including any unconverted CFC-216ba and CFC-216aa, may be recovered and returned to step (a) or returned to the hydrogenation reactor in step (b).

Embodiments of this invention include, but are not limited to:

Embodiment D1. A process for the manufacture of 1,1,1,3,3,3-hexafluoropropane and at least one compound selected from the group consisting of 1,1,1,2,3,3-hexafluoropropane and hexafluoropropene, comprising (a) reacting HF, C12, and at least one halopropene of the formula CX₃CCl═CClX, wherein each X is independently selected from the group consisting of F and Cl, to produce a product comprising CF₃CCl₂CF₃ and CF₃CClFCClF₂, wherein said CF₃CCl₂CF₃ and CF₃CClFCClF₂ are produced in the presence of a catalyst composition comprising chromium, oxygen, and at least two modifier metals selected from the group consisting of gold, silver, and palladium as essential constituent elements, wherein the total amount of modifier metals in said catalyst composition is from about 0.05 atom % to about 10 atom % based on the total amount of chromium and modifier metals in the catalyst composition; (b) reacting CF₃CCl₂CF₃ and CF₃CClFCClF₂ produced in (a) with hydrogen, optionally in the presence of HF, to produce a product comprising CF₃CH₂CF₃ and at least one compound selected from the group consisting of CHF₂CHFCF₃, CF₃CF═CF₂ and CF₃CFHCF₃; and (c) recovering from the product produced in (b), CF₃CH₂CF₃ and at least one compound selected from the group consisting of CHF₂CHFCF₃, CF₃CF═CF₂ and CF₃CFHCF₃.

Embodiment D2. The process of Embodiment D1 wherein the halopropene reactant is contacted with Cl₂ and HF in a pre-reactor.

Embodiment D3. The process of Embodiment D1 wherein the halopropene reactant is contacted with HF in a pre-reactor.

Embodiment D4. The process of Embodiment D1 wherein the reaction of (b) is conducted in a reaction zone at a temperature of from about 350° C. to about 800° C. which is unpacked or packed with a nickel alloy.

Embodiment D5. The process of Embodiment D1 wherein the reaction of (b) is conducted in a reaction zone at a temperature of from about 100° C. to about 350° C. containing a hydrogenation catalyst.

Embodiment D6. The process of Embodiment D1 wherein the amount of modifier metals relative to the total amount of chromium and modifier metals in the catalyst composition is from about 0.5 atom % to about 5 atom %.

Embodiment D7. The process of Embodiment D1 wherein the catalyst composition further comprises fluorine as an essential constituent element.

Embodiment D8. The process of Embodiment D1 wherein the catalyst composition comprises alpha-chromium oxide, metallic gold and metallic silver.

Embodiment D9. The process of Embodiment D1 wherein the catalyst composition comprises alpha-chromium oxide, metallic gold and palladium.

Embodiment D10. The process of Embodiment D1 wherein the catalyst composition comprises alpha-chromium oxide, metallic silver and palladium.

Embodiment D11. The process of Embodiment D1 wherein the catalyst composition comprises alpha-chromium oxide, metallic gold, metallic silver and palladium.

Embodiment D12. The process of Embodiment D1 wherein the catalyst composition comprises silver and gold and the mole ratio of silver to gold is from about 10:1 to about 1:10.

Embodiment D13. The process of Embodiment D1 wherein the catalyst composition comprises silver and palladium and the mole ratio of silver to palladium is from about 10:1 to about 1:10.

Embodiment D14. The process of Embodiment D1 wherein the catalyst composition comprises palladium and gold and the mole ratio of palladium to gold is from about 10:1 to about 1:10.

### Examples

Reference is made to Examples A6-A10 in Invention Category A above for the chlorofluorination of CFC-1213xa.

Examination of the data shown in Table A2 above shows that the fluorine content of the starting CFC-1213xa is increased to produce CFC-216aa and CFC-216ba as well as other useful products containing a higher fluorine content than the starting material by using the catalysts of this invention. The CF₃CCl₂CF₃ and CF₃CClFCClF₂ may be hydrogenated to produce a mixture of CF₃CH₂CF₃ and at least one of CHF₂CHFCF₃ and CF₃CF═CF₂ from which CF₃CH₂CF₃ and at least one compound selected from the group consisting of CHF₂CHFCF₃, CF₃CF═CF₂ and CF₃CFHCF₃ may be recovered using procedures known to the art.

### E.

Invention Category E of this application provides a process for the preparation of CF₃CH═CF₂ (HFC-1225zc) and/or CF₃CF═CHF (HFC-1225ye). The HFC-1225zc and HFC-1225ye may be recovered as individual products and/or as one or more mixtures of the two products. HFC-1225ye as used herein refers to the isomers, E-HFC-1225ye (CAS Reg No. [5595-10-8]) or Z-HFC-1225ye (CAS Reg. No. [5528-43-8]), as well as any combinations or mixtures of such isomers.

In step (a) of the process of this invention, one or more halopropene starting materials CX₃CCl═CClX, wherein each X is independently selected from the group consisting of F and Cl, are reacted with chlorine (Cl₂) and hydrogen fluoride (HF) to produce a product mixture comprising CF₃CCl₂CF₃ (CFC-216aa) and CF₃CClFCClF₂ (CFC-216ba). Accordingly, this invention also provides a process for the preparation of mixtures of CF₃CCl₂CF₃ (CFC-216aa) and CF₃CClFCClF₂ (CFC-216ba) from readily available starting materials.

Suitable starting materials for the process of this invention include E- and Z-CF₃CCl═CClF (CFC-1214xb), CF₃CCl═CCl₂ (CFC-1213xa), CClF₂CCl═CCl₂ (CFC-1212xa), CCl₂FCCl═CCl₂ (CFC-1211xa), and CCl₃CCl═CCl₂ (hexachloropropene, HCP), or mixtures thereof.

Due to their availability, CF₃CCl═CCl₂ (CFC-1213xa) and CCl₃CCl═CCl₂ (hexachloropropene, HCP) are the preferred halopropene starting materials for the process of the invention.

Preferably, the reaction of HF and Cl₂ with CX₃CCl═CClX is carried out in the vapor phase in a heated tubular reactor. A number of reactor configurations are possible, including vertical and horizontal orientation of the reactor and different modes of contacting the halopropene starting material(s) with HF and chlorine. Preferably the HF and chlorine are substantially anhydrous.

In one embodiment of step (a), the halopropene starting material(s) are fed to the reactor containing the chlorofluorination catalyst. The halopropene starting material(s) may be initially vaporized and fed to the reaction zone as gas(es).

In another embodiment of step (a), the halopropene starting material(s) may be contacted with HF in a pre-reactor (i.e. prior to contacting the chlorofluorination catalysts). The pre-reactor may be empty (i.e., unpacked), but is preferably filled with a suitable packing such as Monel™ or Hastelloy™ nickel alloy turnings or wool, or other material inert to HCl and HF, which allows for efficient mixing of CX₃CCl═CClX and HF vapor.

If the halopropene starting material(s) are fed to the pre-reactor as liquid(s), it is preferable for the pre-reactor to be oriented vertically with CX₃CCl═CClX entering the top of the reactor and pre-heated HF vapor introduced at the bottom of the reactor.

Suitable temperatures for the pre-reactor are within the range of from about 80° C. to about 250° C., preferably from about 100° C. to about 200° C. Under these conditions, for example, hexachloropropene is converted to a mixture containing predominantly CFC-1213xa. The feed rate of the starting material is determined by the length and diameter of the reactor, reactor temperature, and the degree of fluorination desired in the pre-reactor. Slower feed rates at a given temperature will increase contact time and tend to increase the amount of conversion of the starting material and increase the degree of fluorination of the products.

The term “degree of fluorination” means the extent to which fluorine atoms replace chlorine substituents in the CX₃CCl═CClX starting materials. For example, CF₃CCl═CClF represents a higher degree of fluorination than CClF₂CCl═CCl₂ and CF₃CCl₂CF₃ represents a higher degree of fluorination than CClF₂CCl₂CF₃.

The molar ratio of HF fed to the pre-reactor, or otherwise to the reaction zone of step (a), to halopropene starting material fed in step (a) is typically from about stoichiometric to about 50:1. The stoichiometric ratio depends on the average degree of fluorination of the halopropene starting material(s) and is typically based on formation of C₃Cl₂F₆ isomers. For example, if the halopropene is HCP, the stoichiometric ratio of HF to HCP is 6:1; if the halopropene is CFC-1213xa, the stoichiometric ratio of HF to CFC-1213xa is 3:1. Preferably, the molar ratio of HF to halopropene starting material is from about twice the stoichiometric ratio (based on formation of C₃Cl₂F₆ isomers) to about 30:1. Higher ratios of HF to halopropene are not particularly beneficial. Lower ratios result in reduced yields of C₃Cl₂F₆ isomers.

If the halopropene starting materials are contacted with HF in a pre-reactor, the effluent from the pre-reactor is then contacted with chlorine in the reaction zone of step (a).

In another embodiment of the invention, the halopropene starting material(s) may be contacted with Cl₂ and HF in a pre-reactor (i.e. prior to contacting the chlorofluorination catalyst). The pre-reactor may be empty (i.e., unpacked) but is preferably filled with a suitable packing such as Monel™ or Hastelloy™ nickel alloy turnings or wool, activated carbon, (or other material inert to HCl, HF, and Cl₂) which allows for efficient mixing of CX₃CCl═CClX, HF, and Cl₂.

Typically, at least a portion of the halopropene starting material(s) react(s) with Cl₂ and HF in the pre-reactor by addition of Cl₂ to the olefinic bond to give a saturated halopropane as well as by substitution of at least a portion of the Cl substituents in the halopropropane and/or halopropene by F. Suitable temperatures for the pre-reactor in this embodiment of the invention are within the range of from about 80° C. to about 250° C., preferably from about 100° C. to about 200° C. Higher temperatures result in greater conversion of the halopropene(s) entering the reactor to saturated products and greater degrees of halogenation and fluorination in the pre-reactor products.

The term “degree of halogenation” means the extent to which hydrogen substituents in a halocarbon have been replaced by halogen and the extent to which carbon-carbon double bonds have been saturated with halogen. For example, CF₃CCl₂CClF₂ has a higher degree of halogenation than CF₃CCl═CCl₂. Also, CF₃CCl₂CClF₂ has a higher degree of halogenation than CF₃CHClCClF₂.

The molar ratio of Cl₂ to halopropene starting material(s) in the pre-reactor is typically from about 1:1 to about 10:1, and is preferably from about 1:1 to about 5:1. Feeding Cl₂ at less than a 1:1 ratio will result in the presence of relatively large amounts of unsaturated materials and hydrogen-containing side products in the reactor effluent.

In a preferred embodiment of step (a), the halopropene starting materials are vaporized, preferably in the presence of HF, and contacted with HF and Cl₂ in a pre-reactor and then contacted with the chlorofluorination catalyst. If the preferred amounts of HF and Cl₂ are fed to the pre-reactor, additional HF and Cl₂ are not required in the reaction zone.

Suitable temperatures in the reaction zone(s) of step (a) are within the range of from about 200° C. to about 400° C., preferably from about 250° C. to about 350° C., depending on the desired conversion of the starting material and the activity of the catalyst. Reactor temperatures greater than about 350° C. may result in products having a degree of fluorination greater than five. In other words, at higher temperatures, substantial amounts of chloropropanes containing six or more fluorine substituents (e.g., CF₃CCl₂CF₃ or CF₃CClFCClF₂) may be formed. Reactor temperatures below about 240° C. may result in a substantial yield of products with a degree of fluorination less than five (i.e., underfluorinates).

Suitable reactor pressures for vapor phase embodiments of this invention may be in the range of from about 1 to about 30 atmospheres. Reactor pressures of about 5 atmospheres to about 20 atmospheres may be advantageously employed to facilitate separation of HCl from other reaction products in step (b) of the process.

The chlorofluorination catalysts used in step (a) of this invention comprise chromium, oxygen and modifier metals (e.g., modifier metal-containing chromium oxide) and contain from about 0.05 atom % to about 10 atom % modifier metals based on the total amount of modifier metals and chromium in the catalyst composition. Of note are compositions comprising silver and gold wherein the mole ratio of silver to gold is from about 10:1 to about 1:10. Also of note are compositions comprising silver and palladium wherein the mole ratio of silver to palladium is from about 10:1 to about 1:10. Also of note are compositions comprising palladium and gold wherein the mole ratio of palladium to gold is from about 10:1 to about 1:10.

In one embodiment of this invention, the catalyst composition comprises alpha-chromium oxide (i.e., □-Cr₂O₃) and metallic gold (i.e., gold in the zero oxidation state) and metallic silver (i.e., silver in the zero oxidation state). In another embodiment of this invention, the catalyst composition comprises alpha-chromium oxide (i.e., □-Cr₂O₃) and metallic gold (i.e., gold in the zero oxidation state) and palladium. In another embodiment of this invention, the catalyst composition comprises alpha-chromium oxide (i.e., □-Cr₂O₃) and metallic silver (i.e., silver in the zero oxidation state) and palladium. In another embodiment of this invention, the catalyst composition comprises alpha-chromium oxide (i.e., □-Cr₂O₃), metallic gold (i.e., gold in the zero oxidation state), metallic silver (i.e., silver in the zero oxidation state), and palladium. Of note are embodiments wherein at least 50 weight % of the chromium component is present as alpha-chromium oxide. Also of note are embodiments wherein the gold component consists essentially of metallic gold having an average particle size of from about 1 nanometer to about 500 nanometers. In certain embodiments of this invention, particles of metallic gold and at least one of palladium and metallic silver are dispersed in a matrix comprising chromium oxide. In certain embodiments of this invention, particles of metallic silver and palladium are dispersed in a matrix comprising chromium oxide.

In certain embodiments of this invention, particles of metallic gold and at least one of palladium and metallic silver are supported on a chromium oxide support. In some embodiments, particles of metallic silver and palladium are supported on a chromium oxide support.

The catalyst compositions of this invention may further comprise fluorine as an essential constituent element.

The amount of modifier metals relative to the total amount of chromium and modifier metals in the catalyst compositions used for the chlorofluorination reaction is preferably from about 0.5 atom % to about 5 atom %.

Further information on catalyst compositions comprising chromium, oxygen, and at least two modifier metals selected from the group consisting of gold, silver, and palladium as essential constituent elements useful for this invention (including embodiments further comprising fluorine) is provided in Invention Category A above and in U.S. Patent Applications 60/903,218 [FL 1356 US PRV] filed Feb. 23, 2007, and 60/927,846 [FL 1356 US PRV1] filed May 4, 2007, hereby incorporated herein by reference in their entirety.

The modifier metal-containing chromium oxide catalysts used in the present invention can be formed into various shapes such as pellets, granules, and extrudates for use in packing reactors. They can also be used in powder forms.

The catalyst compositions used in step (a) of this invention may further comprise one or more additives in the form of metal compounds. Such additives may alter the selectivity and/or activity of the modifier metal-containing chromium oxide catalyst compositions or the fluorinated modifier metal-containing chromium oxide catalyst compositions. Suitable additives can be selected from the group consisting of the fluorides, oxides, and oxyfluoride compounds of Mg, Ca, Sc, Y, La, Ti, Zr, W, Mn, Re, Fe, Ru, Co, Rh, Ir, Ni, Pt, Ce, and Zn.

The total content of the additive(s) in the catalyst compositions used in step (a) of the present invention may be from about 0.05 weight % to about 10 weight % based on the total metal content of the catalyst compositions. The additives may be incorporated into the catalyst compositions by standard procedures such as by impregnation or during co-precipitation of the modifier metal and chromium salts.

The catalyst compositions used in step (a) of the present invention can be treated with a fluorinating agent to form catalyst compositions comprising chromium, oxygen, modifier metals and fluorine as essential elements. Typically, prior to use as catalysts, the catalyst compositions are pre-treated with a fluorinating agent. Typically this fluorinating agent is HF though other materials may be used such as sulfur tetrafluoride, carbonyl fluoride, and fluorinated hydrocarbon compounds such as trichlorofluoromethane, dichlorodifluoromethane, chlorodifluoromethane, trifluoromethane, and 1,1,2-trichlorotrifluoroethane. This pretreatment can be accomplished, for example, by placing the catalyst composition in a suitable container which can also be the reactor to be used to perform the process in the present invention, and thereafter, passing HF over the calcined catalyst composition so as to partially saturate the catalyst composition with HF. This can be conveniently carried out by passing HF over the catalyst composition for a period of time, for example, about 0.1 to about 10 hours at a temperature of, for example, about 200° C. to about 450° C. Nevertheless, this pre-treatment is not essential.

Compounds that are produced in the chlorofluorination process step (a) include the halopropanes CF₃CCl₂CF₃ (CFC-216aa) and CF₃CClFCClF₂ (CFC-216ba).

Halopropane by-products that have a higher degree of fluorination than CFC-216aa and CFC-216ba that may be produced in step (a) include CF₃CClFCF₃ (CFC-217ba) and CF₃CF₂CF₃ (FC-218).

Halopropane and halopropene by-products that may be formed in step (a) which have lower degrees of fluorination and/or halogenation than CFC-216aa and CFC-216ba include CF₃CCl₂CClF₂ (CFC-215aa), CF₃CClFCCl₂F (CFC-215bb), CF₃CCl₂CCl₂F (CFC-214ab), and CF₃CCl═CF₂ (CFC-1215xc).

Prior to step (b), the CF₃CCl₂CF₃ and CF₃CClFCClF₂, (and optionally HF) in the effluent from the reaction zone in step (a), are typically separated from the low boiling components of the effluent (which typically comprise HCl, Cl₂, HF, and overfluorinated products such as CF₃CClFCF₃) and the underfluorinated components (which typically comprise C₃Cl₃F₅ (e.g., CFC-215aa and CFC-215bb) isomers, C₃Cl₄F₄ isomers, and/or underhalogenated components such as C₃Cl₂F₄ isomers and CF₃CCl═CCl₂). The higher boiling components may be returned to step (a).

In one embodiment of this invention, the underfluorinated components CFC-215aa and CFC-215bb are converted to CF₃CH₂CHF₂ (HFC-245fa) and CF₃CHFCH₂F (HFC-245eb) as disclosed in U.S. Patent Application 60/927,848 [FL-1365 US PRV] filed May 4, 2007 and hereby incorporated herein by reference.

In another embodiment of this invention, the reactor effluent from step (a) is delivered to a first distillation column in which HCl and any HCl azeotropes are removed from the top of the column while the higher boiling components are removed from the bottom of the column. The products recovered from the bottom of the first distillation column are then delivered to a second distillation column in which HF, Cl₂, and any CFC-217ba are recovered at the top of the second distillation column and remaining HF and organic products, comprising CF₃CCl₂CF₃ and CF₃CClFCClF₂, are recovered at the bottom of the second distillation column. The products recovered from the bottom of the second distillation column may be delivered to further distillation columns or may be delivered to a decanter controlled at a suitable temperature to permit separation of an organic-rich phase and an HF-rich phase. The HF-rich phase may be distilled to recover HF that is then recycled to step (a). The organic-rich phase may then be delivered to step (b).

In step (b) of the process of this invention, CF₃CCl₂CF₃ and CF₃CClFCClF₂ are contacted with hydrogen (H₂), optionally in the presence of HF, in a second reaction zone. The CF₃CCl₂CF₃ and CF₃CClFCClF₂ may be fed to the reaction zone at least in part as their azeotropes with HF.

In one embodiment of step (b), a mixture comprising CF₃CCl₂CF₃ and CF₃CClFCClF₂, and optionally containing HF, is delivered in the vapor phase, along with hydrogen, to a reactor fabricated from nickel, iron, titanium, or their alloys, as described in U.S. Pat. No. 6,540,933; the teachings of this disclosure are incorporated herein by reference.

The temperature of the reaction in this embodiment of step (b) can be between about 350° C. to about 800° C., and is preferably at least about 450° C. Of note are processes wherein the reaction of (b) is conducted in a reaction zone at a temperature of from about 350° C. to about 600° C. which is unpacked or packed with a nickel alloy.

The molar ratio of hydrogen to the CFC-216aa/CFC-216ba mixture fed to the reaction zone should be in the range of about 0.1 mole H₂ per mole of CFC-216 isomer to about 60 moles of H₂ per mole of CFC-216 isomer, more preferably from about 0.4 to 10 moles of H₂ per mole of CFC-216 isomer.

Alternatively, the contacting of hydrogen with the mixture of CFC-216aa and CFC-216ba, and optionally HF, is carried out in the presence of a hydrogenation catalyst. In this embodiment of step (b), said mixture is delivered in the vapor phase, along with hydrogen, to the reaction zone containing a hydrogenation catalyst according to the teachings disclosed in U.S. Patent Application No. 60/706,161 filed on Aug. 5, 2005 and incorporated herein by reference. Hydrogenation catalysts suitable for use in this embodiment include catalysts comprising at least one metal selected from the group consisting of iron, ruthenium, rhodium, iridium, palladium, and platinum. Said catalytic metal component is typically supported on a carrier such as carbon or graphite or a metal oxide, fluorinated metal oxide, or metal fluoride where the carrier metal is selected from the group consisting of magnesium, aluminum, titanium, vanadium, chromium, iron, and lanthanum. Preferred catalysts for the hydrogenolysis include palladium supported on fluorided alumina or carbon. The hydrogenolysis of saturated acyclic halofluorocarbons containing 3 or 4 carbon atoms using palladium supported on carbon is disclosed in U.S. Pat. No. 5,523,501, the teachings of which are incorporated herein by reference.

The supported metal catalysts may be prepared by conventional methods known in the art such as by impregnation of the carrier with a soluble salt of the catalytic metal (e.g., palladium chloride or rhodium nitrate) as described by Satterfield on page 95 of Heterogenous Catalysis in Industrial Practice, 2^{nd} edition (McGraw-Hill, New York, 1991). The concentration of the catalytic metal(s) on the support is typically in the range of about 0.1% by weight of the catalyst to about 5% by weight.

Suitable temperatures for the reaction zone containing said hydrogenation catalyst are in the range of from about 100° C. to about 350° C., preferably from about 125° C. to about 300° C. Higher temperatures typically result in greater conversion of CFC-216aa and CFC-216ba with fewer partially chlorinated intermediates such as C₃HClF₆ isomers.

The amount of hydrogen (H₂) fed to the reaction zone containing said hydrogenation catalyst is typically from about 1 mole of H₂ per mole of dichlorohexafluoropropane to about 20 moles of H₂ per mole of dichlorohexafluoropropane, preferably from about 2 moles of H₂ per mole of dichlorohexafluoropropane to about 10 moles of H₂ per mole of dichlorohexafluoropropane.

The pressure used in the step (b) reaction zone is not critical and may be in the range of from about 1 to 30 atmospheres. A pressure of about 20 atmospheres may be advantageously employed to facilitate separation of HCl from other reaction products.

The effluent from the step (b) reaction zone typically includes HCl, unreacted hydrogen, CF₃CF═CF₂ (HFP), CF₃CH₂CF₃ (HFC-236fa) and CF₃CHFCHF₂ (HFC-236ea), as well as any HF carried over from step (a) or step (b). In addition, small amounts of CF₃CF₂CH₂F (HFC-236cb), CF₃CCl═CF₂ (CFC-1215xc), and partially chlorinated by-products such as C₃HClF₆ isomers including CF₃CHClCF₃ (HCFC-226da), CF₃CClFCHF₂ (HCFC-226ba), CF₃CHFCClF₂ (HCFC-226ea), may be formed.

In one embodiment of this invention, the reactor effluent from step (b) may be delivered to a separation unit (e.g., distillation) to isolate CF₃CH₂CF₃ and CF₃CHFCHF₂, typically as a mixture. CF₃CF═CF₂ may be recovered from the step (b) effluent as a separate product.

In step (c) of the process of this invention, CF₃CH₂CF₃ and CF₃CHFCHF₂ produced in step (b) are dehydrofluorinated.

In one embodiment of step (c), a mixture comprising CF₃CH₂CF₃ and CF₃CHFCHF₂, and optionally an inert gas, is delivered in the vapor phase to a dehydrofluorination catalyst as described in U.S. Pat. No. 6,369,284; the teachings of this disclosure are incorporated herein by reference. Dehydrofluorination catalysts suitable for use in this embodiment include (1) at least one compound selected from the oxides, fluorides and oxyfluorides of magnesium, zinc and mixtures of magnesium and zinc, (2) lanthanum oxide, (3) fluorided lanthanum oxide, (4) activated carbon, and (5) three-dimensional matrix carbonaceous materials.

The catalytic dehydrofluorination of CF₃CH₂CF₃ and CF₃CHFCHF₂ is suitably conducted at a temperature in the range of from about 200° C. to about 500° C., and preferably from about 350° C. to about 450° C. The contact time is typically from about 1 to about 450 seconds, preferably from about 10 to about 120 seconds.

The reaction pressure can be subatmospheric, atmospheric or superatmospheric. Generally, near atmospheric pressures are preferred. However, the dehydrofluorination of CF₃CH₂CF₃ and CF₃CHFCHF₂ can be beneficially run under reduced pressure (i.e., pressures less than one atmosphere).

The catalytic dehydrofluorination can optionally be carried out in the presence of an inert gas such as nitrogen, helium or argon. The addition of an inert gas can be used to increase the extent of dehydrofluorination. Of note are processes wherein the mole ratio of inert gas to CF₃CH₂CF₃ and/or CF₃CHFCHF₂ is from about 5:1 to 1:1. Nitrogen is the preferred inert gas.

The products from the step (c) reaction zone typically include HF, E- and Z-forms of CF₃CF═CHF (HFC-1225ye), CF₃CH═CF₂ (HFC-1225zc), CF₃CH₂CF₃, CF₃CHFCHF₂ and small amounts of other products. Unconverted CF₃CH₂CF₃ and CF₃CHFCHF₂ are recycled back to the dehydrofluorination reactor to produce additional quantities of CF₃CF═CHF and CF₃CH═CF₂.

In another embodiment of step (c), the CF₃CH₂CF₃ and CF₃CHFCHF₂ are subjected to dehydrofluorination at an elevated temperature in the absence of a catalyst by using procedures similar to those disclosed in U.S. Patent Application Publication No. 2006/0094911 which is incorporated herein by reference. The reactor can be fabricated from nickel, iron, titanium, or their alloys, as described in U.S. Pat. No. 6,540,933; the teachings of this disclosure are incorporated herein by reference.

The temperature of the reaction in this embodiment can be between about 350° C. and about 900° C., and is preferably at least about 450° C.

In yet another embodiment of step (c), the CF₃CH₂CF₃ and CF₃CHFCHF₂ are dehydrofluorinated by reaction with caustic (e.g. KOH) using procedures known to the art.

In step (d) of the process of this invention, CF₃CH═CF₂, CF₃CF═CHF, or both CF₃CH═CF₂ and CF₃CF═CHF produced in (c) are recovered individually and/or as one or more mixtures of CF₃CH═CF₂ and CF₃CF═CHF by well known procedures such as distillation.

Embodiments of this invention include, but are not limited to:

Embodiment E1. A process for the manufacture of at least one compound selected from the group consisting of 1,1,3,3,3-pentafluoropropene and 1,2,3,3,3-pentafluoropropene, comprising (a) reacting hydrogen fluoride, chlorine, and at least one halopropene of the formula CX₃CCl═CClX, wherein each X is independently selected from the group consisting of F and Cl, to produce a product comprising CF₃CCl₂CF₃ and CF₃CClFCClF₂, wherein said CF₃CCl₂CF₃ and CF₃CClFCClF₂ are produced in the presence of a catalyst composition comprising chromium, oxygen, and at least two modifier metals selected from the group consisting of gold, silver, and palladium as essential constituent elements, wherein the total amount of modifier metals in said catalyst composition is from about 0.05 atom % to about 10 atom % based on the total amount of chromium and modifier metals in the catalyst composition; (b) reacting CF₃CCl₂CF₃ and CF₃CClFCClF₂ produced in (a) with hydrogen, optionally in the presence of hydrogen fluoride, to produce a product comprising CF₃CH₂CF₃ and CF₃CHFCHF₂; (c) dehydrofluorinating CF₃CH₂CF₃ and CF₃CHFCHF₂ produced in (b) to produce a product comprising CF₃CH═CF₂ and CF₃CF═CHF; and (d) recovering at least one compound selected from the group consisting of CF₃CH═CF₂ and CF₃CF═CHF from the product produced in (c).

Embodiment E2. The process of Embodiment E1 wherein the halopropene reactant is contacted with Cl₂ and HF in a pre-reactor.

Embodiment E3. The process of Embodiment E1 wherein the halopropene reactant is contacted with HF in a pre-reactor.

Embodiment E4. The process of Embodiment E1 wherein the reaction of (b) is conducted in a reaction zone at a temperature of from about 350° C. to about 800° C. which is unpacked or packed with a nickel alloy.

Embodiment E5. The process of Embodiment E1 wherein the reaction of (b) is conducted in a reaction zone at a temperature of from about 100° C. to about 350° C. containing a hydrogenation catalyst.

Embodiment E6. The process of Embodiment E1 wherein the reaction of (c) is conducted in the absence of a catalyst at a temperature of from about 350° C. to about 900° C.

Embodiment E7. The process of Embodiment E1 wherein the reaction of (c) is conducted in a reaction zone containing a dehydrofluorination catalyst at a temperature of from about 200° C. to about 500° C.

Embodiment E8. The process of Embodiment E1 wherein the amount of modifier metals relative to the total amount of chromium and modifier metals in the catalyst composition is from about 0.5 atom % to about 5 atom %.

Embodiment E9. The process of Embodiment E1 wherein the catalyst composition further comprises fluorine as an essential constituent element.

Embodiment E10. The process of Embodiment E1 wherein the catalyst composition comprises alpha-chromium oxide, metallic gold and metallic silver.

Embodiment E11. The process of Embodiment E1 wherein the catalyst composition comprises alpha-chromium oxide, metallic gold and palladium.

Embodiment E12. The process of Embodiment E1 wherein the catalyst composition comprises alpha-chromium oxide, metallic silver and palladium.

Embodiment E13. The process of Embodiment E1 wherein the catalyst composition comprises alpha-chromium oxide, metallic gold, metallic silver and palladium.

Embodiment E14. The process of Embodiment E1 wherein the catalyst composition comprises silver and gold and the mole ratio of silver to gold is from about 10:1 to about 1:10.

Embodiment E15. The process of Embodiment E1 wherein the catalyst composition comprises silver and palladium and the mole ratio of silver to palladium is from about 10:1 to about 1:10.

Embodiment E16. The process of Embodiment E1 wherein the catalyst composition comprises palladium and gold and the mole ratio of palladium to gold is from about 10:1 to about 1:10.

### Examples

Reference is made to Examples A6-A10 in Invention Category A above for the chlorofluorination of CFC-1213xa.

Examination of the data shown in Table A2 above shows that the amount of CFC-216aa and CFC-216ba can be maximized relative to CFC-215aa and CFC-215bb by controlling the operational variables and by using the catalysts of this invention. The CFC-216aa and CFC-216ba produced above may be hydrogenated to produce HFC-236fa and HFC-236ea, respectively, in a manner analogous to the teachings of International Publication No. WO 2005/037743 A1 and U.S. Pat. No. 5,523,501. The HFC-236fa and HFC-236ea may be dehydrofluorinated to HFC-1225zc and HFC-1225ye, respectively, in accordance with the teachings described in U.S. Pat. No. 6,369,284. The HFC-1225zc and HFC-1225ye may be recovered individually or as mixtures of HFC-1225zc and HFC-1225ye by procedures known to the art.

### F.

Invention Category F of this application provides a process for the preparation of CF₃CH₂CHF₂ (HFC-245fa), CF₃CH₂CF₃ (HFC-236fa), or both CF₃CH₂CHF₂ and CF₃CH₂CF₃. The HFC-245fa and HFC-236fa may be recovered as individual products and/or as one or more mixtures of the two products.

In step (a) of the process of this invention, one or more halopropene compounds of the formula CX₃CCl═CClX, wherein each X is independently selected from the group consisting of F and Cl, are reacted with hydrogen fluoride (HF) to produce a product mixture comprising at least one of CF₃CCl═CF₂ (CFC-1215xc) and CF₃CHClCF₃ (HCFC-226da). Accordingly, this invention provides a process for the preparation of at least one of CF₃CCl═CF₂ and CF₃CHClCF₃ from readily available starting materials.

Suitable starting materials for the process of this invention include E- and Z-CF₃CCl═CClF (CFC-1214xb), CF₃CCl═CCl₂ (CFC-1213xa), CClF₂CCl═CCl₂ (CFC-1212xa), CCl₂FCCl═CCl₂ (CFC-1211xa), and CCl₃CCl═CCl₂ (hexachloropropene, HCP), or mixtures thereof.

Due to their availability, CF₃CCl═CCl₂ (CFC-1213xa) and CCl₃CCl═CCl₂ (hexachloropropene, HCP) are the preferred starting materials for the process of the invention.

Preferably, the reaction of HF with CX₃CCl═CClX is carried out in the vapor phase in a heated tubular reactor. A number of reactor configurations are possible, including vertical and horizontal orientation of the reactor and different modes of contacting the halopropene starting material(s) with HF. Preferably the HF is substantially anhydrous.

In one embodiment of step (a), the halopropene starting material(s) and HF may be fed to the reactor containing the fluorination catalyst. The halopropene starting material(s) may be initially vaporized and fed to the reactor as gas(es).

In another embodiment of step (a), the halopropene starting material(s) may be contacted with HF in a pre-reactor (i.e. prior to contacting the fluorination catalyst). The pre-reactor may be empty (i.e., unpacked), but is preferably filled with a suitable packing such as Monel™ or Hastelloy™ nickel alloy turnings or wool, or other material inert to HCl and HF, for efficient mixing of CX₃CCl═CClX and HF.

If the halopropene starting material(s) are fed to the pre-reactor as liquid(s), it is preferable for the pre-reactor to be oriented vertically with CX₃CCl═CClX entering the top of the reactor and pre-heated HF vapor introduced at the bottom of the reactor.

Suitable temperatures for the pre-reactor are within the range of from about 80° C. to about 250° C., preferably from about 100° C. to about 200° C. Under these conditions, for example, hexachloropropene is converted to a mixture containing predominantly CFC-1213xa. The feed rate of the starting material is determined by the length and diameter of the reactor, reactor temperature, and the degree of fluorination desired in the pre-reactor. Slower feed rates at a given temperature will increase contact time and tend to increase the amount of conversion of the starting material and increase the degree of fluorination of the products.

The term “degree of fluorination” means the extent to which fluorine atoms replace chlorine substituents in the CX₃CCl═CClX starting materials. For example, CF₃CCl═CClF represents a higher degree of fluorination than CClF₂CCl═CCl₂ and CF₃CCl₂CF₃ represents a higher degree of fluorination than CClF₂CCl₂CF₃.

The molar ratio of HF fed to the pre-reactor, or otherwise to the reaction zone of step (a), to halopropene starting material fed in step (a) is typically from about stoichiometric to about 50:1. The stoichiometric ratio depends on the average degree of fluorination of the halopropene starting material(s) and is typically based on formation of C₃ClF₅. For example, if the halopropene is HCP, the stoichiometric ratio of HF to HCP is 5:1; if the halopropene is CFC-1213xa, the stoichiometric ratio of HF to CFC-1213xa is 2:1. Preferably, the molar ratio of HF to halopropene starting material is from about twice the stoichiometric ratio (based on formation of C₃ClF₅) to about 30:1. Higher ratios of HF to halopropene are not particularly beneficial. Lower ratios result in reduced yields of CFC-1215xc and HCFC-226da.

In a preferred embodiment of step (a) the halopropene starting materials are vaporized, preferably in the presence of HF, contacted with HF in a pre-reactor, and then contacted with the fluorination catalyst. If the preferred amount of HF is fed in the pre-reactor, additional HF is not required in the reaction zone(s) of step (a).

Suitable temperatures in the reaction zone(s) of step (a) for catalytic fluorination of halopropene starting materials and/or their products formed in the pre-reactor are within the range of about 200° C. to about 400° C., preferably from about 240° C. to about 350° C., depending on the desired conversion of the starting material and the activity of the catalyst. Higher temperatures typically contribute to reduced catalyst life. Temperatures below about 240° C. may result in substantial amounts of products having a degree of fluorination less than five (i.e., underfluorinates). By adjusting process conditions such as temperature, contact time, and HF ratios, greater or lesser amounts of CFC-1215xc relative to HCFC-226da can be formed.

Suitable reactor pressures for vapor phase embodiments of this invention may be in the range of from about 1 to about 30 atmospheres. Reactor pressures of about 5 atmospheres to about 20 atmospheres may be advantageously employed to facilitate separation of HCl from other reaction products in step (b) of the process.

The fluorination catalysts used in step (a) of this invention comprise chromium, oxygen and modifier metals (e.g., modifier metal-containing chromium oxide) and contain from about 0.05 atom % to about 10 atom % modifier metals based on the total amount of modifier metals and chromium in the catalyst composition. Of note are compositions comprising silver and gold wherein the mole ratio of silver to gold is from about 10:1 to about 1:10. Also of note are compositions comprising silver and palladium wherein the mole ratio of silver to palladium is from about 10:1 to about 1:10. Also of note are compositions comprising palladium and gold wherein the mole ratio of palladium to gold is from about 10:1 to about 1:10.

In one embodiment of this invention, the catalyst composition comprises alpha-chromium oxide (i.e., □-Cr₂O₃) and metallic gold (i.e., gold in the zero oxidation state) and metallic silver (i.e., silver in the zero oxidation state). In another embodiment of this invention, the catalyst composition comprises alpha-chromium oxide (i.e., □-Cr₂O₃) and metallic gold (i.e., gold in the zero oxidation state) and palladium. In another embodiment of this invention, the catalyst composition comprises alpha-chromium oxide (i.e., □-Cr₂O₃) and metallic silver (i.e., silver in the zero oxidation state) and palladium. In another embodiment of this invention, the catalyst composition comprises alpha-chromium oxide (i.e., □-Cr₂O₃), metallic gold (i.e., gold in the zero oxidation state), metallic silver (i.e., silver in the zero oxidation state), and palladium. Of note are embodiments wherein at least 50 weight % of the chromium component is present as alpha-chromium oxide. Also of note are embodiments wherein the gold component consists essentially of metallic gold having an average particle size of from about 1 nanometer to about 500 nanometers. In certain embodiments of this invention, particles of metallic gold and at least one of palladium and metallic silver are dispersed in a matrix comprising chromium oxide. In certain embodiments of this invention, particles of metallic silver and palladium are dispersed in a matrix comprising chromium oxide.

In certain embodiments of this invention, particles of metallic gold and at least one of palladium and metallic silver are supported on a chromium oxide support. In some embodiments, particles of metallic silver and palladium are supported on a chromium oxide support.

The catalyst compositions of this invention may further comprise fluorine as an essential constituent element.

The amount of modifier metals relative to the total amount of chromium and modifier metals in the catalyst compositions used for the fluorination reaction is preferably from about 0.5 atom % to about 5 atom %.

Further information on catalyst compositions comprising chromium, oxygen, and at least two modifier metals selected from the group consisting of gold, silver, and palladium as essential constituent elements useful for this invention (including embodiments further comprising fluorine) is provided in Invention Category A above and in U.S. Patent Applications 60/903,218 [FL 1356 US PRV] filed Feb. 23, 2007, and 60/927,846 [FL 1356 US PRV1] filed May 4, 2007, hereby incorporated herein by reference in their entirety.

The modifier metal-containing chromium oxide catalysts used in the present invention can be formed into various shapes such as pellets, granules, and extrudates for use in packing reactors. They can also be used in powder forms.

The catalyst compositions used in step (a) of this invention may further comprise one or more additives in the form of metal compounds. Such additives may alter the selectivity and/or activity of the modifier metal-containing chromium oxide catalyst compositions or the fluorinated modifier metal-containing chromium oxide catalyst compositions. Suitable additives can be selected from the group consisting of the fluorides, oxides, and oxyfluoride compounds of Mg, Ca, Sc, Y, La, Ti, Zr, W, Mn, Re, Fe, Ru, Co, Rh, Ir, Ni, Pt, Ce, and Zn.

The total content of the additive(s) in the catalyst compositions used in step (a) of the present invention may be from about 0.05 weight % to about 10 weight % based on the total metal content of the catalyst compositions. The additives may be incorporated into the catalyst compositions by standard procedures such as by impregnation or during co-precipitation of the modifier metal and chromium salts.

The catalyst compositions used in step (a) of the present invention can be treated with a fluorinating agent to form catalyst compositions comprising chromium, oxygen, modifier metals and fluorine as essential elements. Typically, prior to use as catalysts, the catalyst compositions are pre-treated with a fluorinating agent. Typically this fluorinating agent is HF though other materials may be used such as sulfur tetrafluoride, carbonyl fluoride, and fluorinated hydrocarbon compounds such as trichlorofluoromethane, dichlorodifluoromethane, chlorodifluoromethane, trifluoromethane, and 1,1,2-trichlorotrifluoroethane. This pretreatment can be accomplished, for example, by placing the calcined catalyst composition in a suitable container which can also be the reactor to be used to perform the process in the present invention, and thereafter, passing HF over the calcined catalyst composition so as to partially saturate the catalyst composition with HF. This can be conveniently carried out by passing HF over the catalyst composition for a period of time, for example, about 0.1 to about 10 hours at a temperature of, for example, about 200° C. to about 450° C. Nevertheless, this pre-treatment is not essential.

Compounds that are produced in the fluorination process step (a) include the CF₃CCl═CF₂ (CFC-1215xc) and CF₃CHClCF₃ (HCFC-226da).

Halopropane by-products having a lower degree of fluorination than HCFC-226da that may be formed in step (a) include CF₃CHClCClF₂ (HCFC-225da). Other halopropane by-products which may be formed include CFC-216aa (CF₃CCl₂CF₃).

Halopropene by-products having a lower degree of fluorination than CFC-1215xc that may be formed in step (a) include E- and Z-CF₃CCl═CClF (CFC-1214xb, C₃Cl₂F₄ isomers) and CF₃CCl═CCl₂ (CFC-1213xa).

Prior to step (b), CFC-1215xc and HCFC-226da (and optionally HF) from the effluent from the reaction zone in step (a), are typically separated from lower boiling components of the effluent (which typically comprise HCl) and the underfluorinated components of the effluent (which typically comprise HCFC-225da, C₃Cl₂F₄ isomers, and CFC-1213xa).

In one embodiment of the invention, the reactor effluent from step (a) may be delivered to a first distillation column in which HCl and any HCl azeotropes are removed from the top of column while the higher boiling components are removed at the bottom of the column. The products recovered at the bottom of the first distillation column are then delivered to a second distillation column in which CF₃CHClCF₃, CF₃CCl═CF₂, and HF, are separated at the top of the column, and any remaining HF and underfluorinated components are removed from the bottom of the column.

The mixture of CF₃CHClCF₃, CF₃CCl═CF₂, and HF recovered from the top of the second distillation column may be delivered to step (b) or may optionally be delivered to a decanter maintained at a suitable temperature to cause separation of an organic-rich liquid phase and an HF-rich liquid phase. The HF-rich phase may be distilled to recover HF that is then recycled to step (a). The organic-rich phase may then be delivered to step (b) or may be processed to produce HCFC-226da and CFC-1215xc individually or as mixture.

In another embodiment of the invention said underfluorinated components such as HCFC-225da, C₃Cl₂F₄ isomers, and CF₃CCl═CCl₂ (CFC-1213xa) may be returned to step (a).

In connection with developing processes for the separation of CFC-1215xc, it is noted that CFC-1215xc can be present as an azeotrope with HF.

Further information on azeotropic compositions of CFC-1215xc and HF is disclosed in U.S. Patent Application 60/927,818 [FL-1339 US PRV], filed May 4, 2007 and hereby incorporated herein by reference.

In step (b) of the process of this invention, the CF₃CHClCF₃ and/or CF₃CCl═CF₂ produced in step (a) are reacted with hydrogen (H₂), optionally in the presence of HF.

In one embodiment of step (b), a mixture comprising CFC-1215xc and/or HCFC-226da produced in step (a), and optionally HF, is delivered in the vapor phase, along with hydrogen (H₂), to a reactor containing a hydrogenation catalyst.

Hydrogenation catalysts suitable for use in this embodiment include catalysts comprising at least one metal selected from the group consisting of iron, ruthenium, rhodium, iridium, palladium, and platinum. Said catalytic metal component is typically supported on a carrier such as carbon or graphite or a metal oxide, fluorinated metal oxide, or metal fluoride where the carrier metal is selected from the group consisting of magnesium, aluminum, titanium, vanadium, chromium, iron, and lanthanum.

Of note are carbon supported catalysts in which the carbon support has been washed with acid and has an ash content below about 0.1% by weight. Hydrogenation catalysts supported on low ash carbon that are suitable for carrying out step (b) of the process of this invention are described in U.S. Pat. No. 5,136,113, the teachings of which are incorporated herein by reference. Also of note are catalysts comprising at least one metal selected from the group consisting of palladium, platinum, and rhodium supported on alumina (Al₂O₃), fluorinated alumina, or aluminum fluoride (AlF₃).

The supported metal catalysts may be prepared by conventional methods known in the art such as by impregnation of the carrier with a soluble salt of the catalytic metal (e.g., palladium chloride or rhodium nitrate) as described by Satterfield on page 95 of Heterogenous Catalysis in Industrial Practice, 2^{nd} edition (McGraw-Hill, New York, 1991). The concentration of the catalytic metal(s) on the support is typically in the range of about 0.1% by weight of the catalyst to about 5% by weight.

The relative amount of hydrogen contacted with CFC-1215xc and HCFC-226da in the presence of the hydrogenation catalyst is typically from about the stoichiometric ratio of hydrogen to CF₃CHClCF₃/CF₃CCl═CF₂ mixture to about 10 moles of H₂ per mole of CF₃CHClCF₃/CF₃CCl═CF₂ mixture. The stoichiometric ratio of hydrogen to the CF₃CHClCF₃/CF₃CCl═CF₂ mixture depends on the relative amounts of the two components in the mixture. The stoichiometric amounts of H₂ required to convert HCFC-226da and CFC-1215xc to CF₃CH₂CF₃ and CF₃CH₂CHF₂, are one and two moles, respectively.

Suitable temperatures for the catalytic hydrogenation are typically from about 100° C. to about 350° C., preferably from about 125° C. to about 300° C. Temperatures above about 350° C. tend to result in defluorination side reactions; temperatures below about 125° C. will result in incomplete substitution of Cl for H in the starting materials. The reactions are typically conducted at atmospheric pressure or superatmospheric pressure.

The effluent from the step (b) reaction zone(s) typically includes HCl, CF₃CH₂CF₃ (HFC-236fa), CF₃CH₂CHF₂ (HFC-245fa), and small amounts of lower boiling by-products (typically including propane, CF₃CH═CF₂ (HFC-1225zc), E- and Z-CF₃CH═CHF (HFC-1234ze), and/or CF₃CH₂CH₃ (HFC-263fb)) and higher boiling by-products and intermediates (typically including CF₃CHFCH₃ (HFC-254eb) and/or CF₃CHClCHF₂ (HCFC-235da)) as well as any unconverted starting materials and any HF carried over from step (a).

In step (c), the desired products are recovered. Products from step (b) may be delivered to a separation unit to recover at least one of CF₃CH₂CF₃ and CF₃CH₂CHF₂ individually, as a mixture, or as their HF azeotropes.

Partially chlorinated components such as HCFC-235da may be recovered and recycled back to step (b).

In one embodiment CF₃CH₂CF₃ and/or CF₃CH₂CHF₂ recovered from step (c) may be dehydrofluorinated to produce CF₃CH═CF₂ and/or E- and Z-CF₃CH═CHF respectively, as disclosed in U.S. Patent Application 60/927,842 [FL-1363 US PRV] filed May 4, 2007 and hereby incorporated herein by reference.

Embodiments of this invention include, but are not limited to:

Embodiment F1. A process for making at least one compound selected from CF₃CH₂CHF₂ and CF₃CH₂CF₃, comprising (a) reacting HF, and at least one halopropene of the formula CX₃CCl═CClX, wherein each X is independently selected from the group consisting of F and Cl, to produce a product comprising at least one compound selected from CF₃CCl═CF₂ and CF₃CHClCF₃, wherein said CF₃CCl═CF₂ and CF₃CHClCF₃ are produced in the presence of a catalyst composition comprising chromium, oxygen, and at least two modifier metals selected from the group consisting of gold, silver and palladium as essential constituent elements, wherein the total amount of modifier metals in said catalyst composition is from about 0.05 atom % to about 10 atom % based on the total amount of chromium and modifier metals in the catalyst composition; (b) reacting at least one compound selected from CF₃CCl═CF₂ and CF₃CHClCF₃ produced in (a) with H₂, optionally in the presence of HF, to produce a product comprising at least one compound selected from CF₃CH₂CHF₂ and CF₃CH₂CF₃; and (c) recovering at least one compound selected from CF₃CH₂CHF₂ and CF₃CH₂CF₃ from the product produced in (b).

Embodiment F2. The process of Embodiment F1 wherein the halopropene reactant is contacted with HF in a pre-reactor.

Embodiment F3. The process of Embodiment F1 wherein the reaction of (b) is conducted in a reaction zone at a temperature of from about 100° C. to about 350° C. containing a hydrogenation catalyst.

Embodiment F4. The process of Embodiment F1 wherein the amount of modifier metals relative to the total amount of chromium and modifier metals in the catalyst composition is from about 0.5 atom % to about 5 atom %.

Embodiment F5. The process of Embodiment F1 wherein the catalyst composition further comprises fluorine as an essential constituent element.

Embodiment F6. The process of Embodiment F1 wherein the catalyst composition comprises alpha-chromium oxide, metallic gold and metallic silver.

Embodiment F7. The process of Embodiment F1 wherein the catalyst composition comprises alpha-chromium oxide, metallic gold and palladium.

Embodiment F8. The process of Embodiment F1 wherein the catalyst composition comprises alpha-chromium oxide, metallic silver and palladium.

Embodiment F9. The process of Embodiment F1 wherein the catalyst composition comprises alpha-chromium oxide, metallic gold, metallic silver and palladium.

Embodiment F10. The process of Embodiment F1 wherein the catalyst composition comprises silver and gold and the mole ratio of silver to gold is from about 10:1 to about 1:10.

Embodiment F11. The process of Embodiment F1 wherein the catalyst composition comprises silver and palladium and the mole ratio of silver to palladium is from about 10:1 to about 1:10.

Embodiment F12. The process of Embodiment F1 wherein the catalyst composition comprises palladium and gold and the mole ratio of palladium to gold is from about 10:1 to about 1:10

### Examples

Reference is made to Examples A1-A5 in Invention Category A above for the chlorofluorination of CFC-1213xa.

Examination of the data in Table A1 above shows that the fluorine content of the starting material CFC-1213xa is increased to produce CFC-1215xc and HCFC-226da that contain a higher fluorine content than the starting material by using the catalysts of this invention.

### G.

Invention Category G of this application provides a process for the manufacture of CF₃CH═CHF (HFC-1234ze), CF₃CH═CF₂ (HFC-1225zc), or both CF₃CH═CHF and CF₃CH═CF₂. The HFC-1234ze and HFC-1225zc may be recovered as individual products and/or as one or more mixtures of the two products. HFC-1234ze may exist as one of two configurational isomers, E or Z. HFC-1234ze as used herein refers to the isomers, E-HFC-1234ze or Z-HFC-1234ze, as well as any combinations or mixtures of such isomers.

In step (a) of the process of this invention, one or more halopropene compounds of the formula CX₃CCl═CClX, wherein each X is independently selected from the group consisting of F and Cl, are reacted with hydrogen fluoride (HF) to produce a product mixture comprising at least one of CF₃CCl═CF₂ (CFC-1215xc) and CF₃CHClCF₃ (HCFC-226da). Accordingly, this invention provides a process for the preparation of at least one of CF₃CCl═CF₂ and CF₃CHClCF₃ from readily available starting materials.

Suitable starting materials for the process of this invention include E- and Z-CF₃CCl═CClF (CFC-1214xb), CF₃CCl═CCl₂ (CFC-1213xa), CClF₂CCl═CCl₂ (CFC-1212xa), CCl₂FCCl═CCl₂ (CFC-1211xa), and CCl₃CCl═CCl₂ (hexachloropropene, HCP), or mixtures thereof.

Due to their availability, CF₃CCl═CCl₂ (CFC-1213xa) and CCl₃CCl═CCl₂ (hexachloropropene, HCP) are the preferred starting materials for the process of the invention.

Preferably, the reaction of HF with CX₃CCl═CClX is carried out in the vapor phase in a heated tubular reactor. A number of reactor configurations are possible, including vertical and horizontal orientation of the reactor and different modes of contacting the halopropene starting material(s) with HF. Preferably the HF is substantially anhydrous.

In one embodiment of step (a), the halopropene starting material(s) and HF may be fed to the reactor containing the fluorination catalyst. The halopropene starting material(s) may be initially vaporized and fed to the reactor as gas(es).

In another embodiment of step (a), the halopropene starting material(s) may be contacted with HF in a pre-reactor (i.e. prior to contacting the fluorination catalyst). The pre-reactor may be empty (i.e., unpacked), but is preferably filled with a suitable packing such as Monel™ or Hastelloy™ nickel alloy turnings or wool, or other material inert to HCl and HF, for efficient mixing of CX₃CCl═CClX and HF.

If the halopropene starting material(s) are fed to the pre-reactor as liquid(s), it is preferable for the pre-reactor to be oriented vertically with CX₃CCl═CClX entering the top of the reactor and pre-heated HF vapor introduced at the bottom of the reactor.

Suitable temperatures for the pre-reactor are within the range of from about 80° C. to about 250° C., preferably from about 100° C. to about 200° C. Under these conditions, for example, hexachloropropene is converted to a mixture containing predominantly CFC-1213xa. The feed rate of the starting material is determined by the length and diameter of the reactor, reactor temperature, and the degree of fluorination desired in the pre-reactor. Slower feed rates at a given temperature will increase contact time and tend to increase the amount of conversion of the starting material and increase the degree of fluorination of the products.

The term “degree of fluorination” means the extent to which fluorine atoms replace chlorine substituents in the CX₃CCl═CClX starting materials. For example, CF₃CCl═CClF represents a higher degree of fluorination than CClF₂CCl═CCl₂ and CF₃CCl₂CF₃ represents a higher degree of fluorination than CClF₂CCl₂CF₃.

The molar ratio of HF fed to the pre-reactor, or otherwise to the reaction zone of step (a), to halopropene starting material fed in step (a) is typically from about stoichiometric to about 50:1. The stoichiometric ratio depends on the average degree of fluorination of the halopropene starting material(s) and is typically based on formation of C₃ClF₅. For example, if the halopropene is HCP, the stoichiometric ratio of HF to HCP is 5:1; if the halopropene is CFC-1213xa, the stoichiometric ratio of HF to CFC-1213xa is 2:1. Preferably, the molar ratio of HF to halopropene starting material is from about twice the stoichiometric ratio (based on formation of C₃ClF₅) to about 30:1. Higher ratios of HF to halopropene are not particularly beneficial. Lower ratios result in reduced yields of CFC-1215xc and HCFC-226da.

In a preferred embodiment of step (a) the halopropene starting materials are vaporized, preferably in the presence of HF, contacted with HF in a pre-reactor, and then contacted with the fluorination catalyst. If the preferred amount of HF is fed in the pre-reactor, additional HF is not required in the reaction zone(s) of step (a).

Suitable temperatures in the reaction zone(s) of step (a) for catalytic fluorination of halopropene starting materials and/or their products formed in the pre-reactor are within the range of about 200° C. to about 400° C., preferably from about 240° C. to about 350° C., depending on the desired conversion of the starting material and the activity of the catalyst. Higher temperatures typically contribute to reduced catalyst life. Temperatures below about 240° C. may result in substantial amounts of products having a degree of fluorination less than five (i.e., underfluorinates). By adjusting process conditions such as temperature, contact time, and HF ratios, greater or lesser amounts of CFC-1215xc relative to HCFC-226da can be formed.

Suitable reactor pressures for vapor phase embodiments of this invention may be in the range of from about 1 to about 30 atmospheres. Reactor pressures of about 5 atmospheres to about 20 atmospheres may be advantageously employed to facilitate separation of HCl from other reaction products in step (b) of the process.

The fluorination catalysts used in step (a) of this invention comprise chromium, oxygen and modifier metals (e.g., modifier metal-containing chromium oxide) and contain from about 0.05 atom % to about 10 atom % modifier metals based on the total amount of modifier metals and chromium in the catalyst composition. Of note are compositions comprising silver and gold wherein the mole ratio of silver to gold is from about 10:1 to about 1:10. Also of note are compositions comprising silver and palladium wherein the mole ratio of silver to palladium is from about 10:1 to about 1:10. Also of note are compositions comprising palladium and gold wherein the mole ratio of palladium to gold is from about 10:1 to about 1:10.

In one embodiment of this invention, the catalyst composition comprises alpha-chromium oxide (i.e., □-Cr₂O₃) and metallic gold (i.e., gold in the zero oxidation state) and metallic silver (i.e., silver in the zero oxidation state). In another embodiment of this invention, the catalyst composition comprises alpha-chromium oxide (i.e., □-Cr₂O₃) and metallic gold (i.e., gold in the zero oxidation state) and palladium. In another embodiment of this invention, the catalyst composition comprises alpha-chromium oxide (i.e., □-Cr₂O₃) and metallic silver (i.e., silver in the zero oxidation state) and palladium. In another embodiment of this invention, the catalyst composition comprises alpha-chromium oxide (i.e., □-Cr₂O₃), metallic gold (i.e., gold in the zero oxidation state), metallic silver (i.e., silver in the zero oxidation state), and palladium. Of note are embodiments wherein at least 50 weight % of the chromium component is present as alpha-chromium oxide. Also of note are embodiments wherein the gold component consists essentially of metallic gold having an average particle size of from about 1 nanometer to about 500 nanometers. In certain embodiments of this invention, particles of metallic gold and at least one of palladium and metallic silver are dispersed in a matrix comprising chromium oxide. In certain embodiments of this invention, particles of metallic silver and palladium are dispersed in a matrix comprising chromium oxide.

In certain embodiments of this invention, particles of metallic gold and at least one of palladium and metallic silver are supported on a chromium oxide support. In some embodiments, particles of metallic silver and palladium are supported on a chromium oxide support.

The catalyst compositions of this invention may further comprise fluorine as an essential constituent element.

The amount of modifier metals relative to the total amount of chromium and modifier metals in the catalyst compositions used for the fluorination reaction is preferably from about 0.5 atom % to about 5 atom %.

Further information on catalyst compositions comprising chromium, oxygen, and at least two modifier metals selected from the group consisting of gold, silver, and palladium as essential constituent elements useful for this invention (including embodiments further comprising fluorine) is provided in Invention Category A above and in U.S. Patent Applications 60/903,218 [FL 1356 US PRV] filed Feb. 23, 2007, and 60/927,846 [FL 1356 US PRV1] filed May 4, 2007, hereby incorporated herein by reference in their entirety.

The modifier metal-containing chromium oxide catalysts used in the present invention can be formed into various shapes such as pellets, granules, and extrudates for use in packing reactors. They can also be used in powder forms.

The catalyst compositions used in step (a) of this invention may further comprise one or more additives in the form of metal compounds. Such additives may alter the selectivity and/or activity of the modifier metal-containing chromium oxide catalyst compositions or the fluorinated modifier metal-containing chromium oxide catalyst compositions. Suitable additives can be selected from the group consisting of the fluorides, oxides, and oxyfluoride compounds of Mg, Ca, Sc, Y, La, Ti, Zr, W, Mn, Re, Fe, Ru, Co, Rh, Ir, Ni, Pt, Ce, and Zn.

The total content of the additive(s) in the catalyst compositions used in step (a) of the present invention may be from about 0.05 weight % to about 10 weight % based on the total metal content of the catalyst compositions. The additives may be incorporated into the catalyst compositions by standard procedures such as by impregnation or during co-precipitation of the modifier metal and chromium salts.

The catalyst compositions used in step (a) of the present invention can be treated with a fluorinating agent to form catalyst compositions comprising chromium, oxygen, modifier metals and fluorine as essential elements. Typically, prior to use as catalysts, the catalyst compositions are pre-treated with a fluorinating agent. Typically this fluorinating agent is HF though other materials may be used such as sulfur tetrafluoride, carbonyl fluoride, and fluorinated hydrocarbon compounds such as trichlorofluoromethane, dichlorodifluoromethane, chlorodifluoromethane, trifluoromethane, and 1,1,2-trichlorotrifluoroethane. This pretreatment can be accomplished, for example, by placing the catalyst composition in a suitable container which can also be the reactor to be used to perform the process in the present invention, and thereafter, passing HF over the calcined catalyst composition so as to partially saturate the catalyst composition with HF. This can be conveniently carried out by passing HF over the catalyst composition for a period of time, for example, about 0.1 to about 10 hours at a temperature of, for example, about 200° C. to about 450° C. Nevertheless, this pre-treatment is not essential.

Compounds that are produced in the fluorination process step (a) include the CF₃CCl═CF₂ (CFC-1215xc) and CF₃CHClCF₃ (HCFC-226da).

Halopropane by-products having a lower degree of fluorination than HCFC-226da that may be formed in step (a) include CF₃CHClCClF₂ (HCFC-225da). Other halopropane by-products which may be formed include CFC-216aa (CF₃CCl₂CF₃).

Halopropene by-products having a lower degree of fluorination than CFC-1215xc that may be formed in step (a) include E- and Z-CF₃CCl═CClF (CFC-1214xb, C₃Cl₂F₄ isomers) and CF₃CCl═CCl₂ (CFC-1213xa).

Prior to step (b), CFC-1215xc and HCFC-226da (and optionally HF) from the effluent from the reaction zone in step (a), are typically separated from lower boiling components of the effluent (which typically comprise HCl) and the underfluorinated components of the effluent (which typically comprise HCFC-225da, C₃Cl₂F₄ isomers, and CFC-1213xa).

In one embodiment of the invention, the reactor effluent from step (a) may be delivered to a first distillation column in which HCl and any HCl azeotropes are removed from the top of column while the higher boiling components are removed at the bottom of the column. The products recovered at the bottom of the first distillation column are then delivered to a second distillation column in which CF₃CHClCF₃, CF₃CCl═CF₂, and HF, are separated at the top of the column, and any remaining HF and underfluorinated components are removed from the bottom of the column.

The mixture of CF₃CHClCF₃, CF₃CCl═CF₂, and HF recovered from the top of the second distillation column may be delivered to step (b) or may optionally be delivered to a decanter maintained at a suitable temperature to cause separation of an organic-rich liquid phase and an HF-rich liquid phase. The HF-rich phase may be distilled to recover HF that is then recycled to step (a). The organic-rich phase may then be delivered to step (b) or may be processed to produce HCFC-226da and CFC-1215xc individually or as a mixture.

In another embodiment of the invention said underfluorinated components such as HCFC-225da, C₃Cl₂F₄ isomers, and CF₃CCl═CCl₂ (CFC-1213xa) may be returned to step (a).

In step (b) of the process of this invention, the CF₃CHClCF₃ and/or CF₃CCl═CF₂ produced in step (a) are reacted with hydrogen (H₂), optionally in the presence of HF.

In one embodiment of step (b), a mixture comprising CFC-1215xc and/or HCFC-226da produced in step (a), and optionally HF, is delivered in the vapor phase, along with hydrogen (H₂), to a reactor containing a hydrogenation catalyst.

Hydrogenation catalysts suitable for use in this embodiment include catalysts comprising at least one metal selected from the group consisting of iron, ruthenium, rhodium, iridium, palladium, and platinum. Said catalytic metal component is typically supported on a carrier such as carbon or graphite or a metal oxide, fluorinated metal oxide, or metal fluoride where the carrier metal is selected from the group consisting of magnesium, aluminum, titanium, vanadium, chromium, iron, and lanthanum.

Of note are carbon supported catalysts in which the carbon support has been washed with acid and has an ash content below about 0.1% by weight. Hydrogenation catalysts supported on low ash carbon that are suitable for carrying out step (b) of the process of this invention are described in U.S. Pat. No. 5,136,113, the teachings of which are incorporated herein by reference. Also of note are catalysts comprising at least one metal selected from the group consisting of palladium, platinum, and rhodium supported on alumina (Al₂O₃), fluorinated alumina, or aluminum fluoride (AlF₃).

The supported metal catalysts may be prepared by conventional methods known in the art such as by impregnation of the carrier with a soluble salt of the catalytic metal (e.g., palladium chloride or rhodium nitrate) as described by Satterfield on page 95 of Heterogenous Catalysis in Industrial Practice, 2^{nd} edition (McGraw-Hill, New York, 1991). The concentration of the catalytic metal(s) on the support is typically in the range of about 0.1% by weight of the catalyst to about 5% by weight.

The relative amount of hydrogen contacted with CFC-1215xc and HCFC-226da in the presence of the hydrogenation catalyst is typically from about the stoichiometric ratio of hydrogen to CF₃CHClCF₃/CF₃CCl═CF₂ mixture to about 10 moles of H₂ per mole of CF₃CHClCF₃/CF₃CCl═CF₂ mixture. The stoichiometric ratio of hydrogen to the CF₃CHClCF₃/CF₃CCl═CF₂ mixture depends on the relative amounts of the two components in the mixture. The stoichiometric amounts of H₂ required to convert HCFC-226da and CFC-1215xc to CF₃CH₂CF₃ and CF₃CH₂CHF₂, are one and two moles, respectively.

Suitable temperatures for the catalytic hydrogenation are typically from about 100° C. to about 350° C., preferably from about 125° C. to about 300° C. Temperatures above about 350° C. tend to result in defluorination side reactions; temperatures below about 125° C. will result in incomplete substitution of Cl for H in the starting materials. The reactions are typically conducted at atmospheric pressure or superatmospheric pressure.

The effluent from the step (b) reaction zone(s) typically includes HCl, CF₃CH₂CF₃ (HFC-236fa), CF₃CH₂CHF₂ (HFC-245fa), and small amounts of lower boiling by-products (typically including propane, CF₃CH═CF₂ (HFC-1225zc), E- and Z-CF₃CH═CHF (HFC-1234ze), and/or CF₃CH₂CH₃ (HFC-263fb)) and higher boiling by-products and intermediates (typically including CF₃CHFCH₃ (HFC-254eb) and/or CF₃CHClCHF₂ (HCFC-235da)) as well as any unconverted starting materials and any HF carried over from step (a).

In one embodiment of step (b), at least one of CF₃CH₂CHF₂ and CF₃CH₂CF₃ produced in step (b) are recovered individually, as a mixture, or as their HF azeotropes as disclosed in U.S. Patent Application 60/927,843 [FL-1362 US PRV] filed May 4, 2007 and hereby incorporated herein by reference.

In step (c) of the process, CF₃CH₂CHF₂ and/or CF₃CH₂CF₃ produced in step (b) are dehydrofluorinated.

In one embodiment of step (c), a mixture comprising CF₃CH₂CHF₂ and CF₃CH₂CF₃, and optionally an inert gas, is delivered in the vapor phase to a reaction zone containing a dehydrofluorination catalyst as described in U.S. Pat. No. 6,369,284; the teachings of this disclosure are incorporated herein by reference.

Dehydrofluorination catalysts suitable for use in this embodiment include (1) at least one compound selected from the oxides, fluorides and oxyfluorides of magnesium, zinc and mixtures of magnesium and zinc, (2) lanthanum oxide, (3) fluorided lanthanum oxide, (4) activated carbon, and (5) three-dimensional matrix carbonaceous materials.

The catalytic dehydrofluorination of CF₃CH₂CHF₂ and CF₃CH₂CF₃ is suitably conducted at a temperature in the range of from about 200° C. to about 500° C., and preferably from about 350° C. to about 450° C. The contact time is typically from about 1 to about 450 seconds, preferably from about 10 to about 120 seconds.

The reaction pressure can be subatmospheric, atmospheric or superatmospheric. Generally, near atmospheric pressures are preferred. However, the dehydrofluorination of CF₃CH₂CHF₂ and CF₃CH₂CF₃ can be beneficially run under reduced pressure (i.e., pressures less than one atmosphere).

The catalytic dehydrofluorination can optionally be carried out in the presence of an inert gas such as nitrogen, helium or argon. The addition of an inert gas can be used to increase the extent of dehydrofluorination. Of note are processes where the mole ratio of inert gas to CF₃CH₂CHF₂ and/or CF₃CH₂CF₃ is from about 5:1 to 1:1. Nitrogen is the preferred inert gas.

The products from the step (c) reaction zone typically include HF, E- and Z-forms of CF₃CH═CHF (HFC-1234ze), CF₃CH═CF₂ (HFC-1225zc), CF₃CH₂CHF₂, CF₃CH₂CF₃, and small amounts of other products. Unconverted CF₃CH₂CHF₂ and CF₃CH₂CF₃ are recycled back to the dehydrofluorination reactor to produce additional quantities of CF₃CF═CHF and CF₃CH═CF₂.

In another embodiment of step (c), the CF₃CH₂CHF₂ and CF₃CH₂CF₃ are subjected to dehydrofluorination at an elevated temperature in the absence of a catalyst following procedures similar to those disclosed in U.S. Patent Application Publication No. 2006/0094911 which is incorporated herein by reference. The reactor can be fabricated from nickel, iron, titanium, or their alloys, as described in U.S. Pat. No. 6,540,933; the teachings of this disclosure are incorporated herein by reference.

The temperature of the reaction in this embodiment can be between about 350° C. and about 900° C., and is preferably at least about 450° C.

In yet another embodiment of step (c), the CF₃CH₂CF₃ and CF₃CH₂CHF₂ are dehydrofluorinated by reaction with caustic (e.g. KOH) using procedures known to the art.

In step (d) of the process, at least one of CF₃CH═CHF and CF₃CH═CF₂ produced in step (c) are recovered individually and/or as one or more mixtures of CF₃CH═CHF and CF₃CH═CF₂ by well known procedures such as distillation.

Embodiments of this invention include, but are not limited to:

Embodiment G1. A process for the manufacture of at least one compound selected from the group consisting of 1,3,3,3-tetrafluoropropene and 1,1,3,3,3-pentafluoropropene, comprising (a) reacting HF, and at least one halopropene of the formula CX₃CCl═CClX, wherein each X is independently selected from the group consisting of F and Cl, to produce a product comprising at least compound selected from CF₃CCl═CF₂ and CF₃CHClCF₃, wherein said CF₃CCl═CF₂ and CF₃CHClCF₃ are produced in the presence of a catalyst composition comprising chromium, oxygen, and at least two modifier metals selected from the group consisting of gold, silver, and palladium as essential constituent elements, wherein the total amount of modifier metals in said catalyst composition is from about 0.05 atom % to about 10 atom % based on the total amount of chromium and modifier metals in the catalyst composition; (b) reacting at least compound selected from CF₃CCl═CF₂ and CF₃CHClCF₃ produced in (a) with H₂, optionally in the presence of HF, to produce a product comprising at least compound selected from CF₃CH₂CHF₂ and CF₃CH₂CF₃; and (c) dehydrofluorinating at least compound selected from CF₃CH₂CHF₂ and CF₃CH₂CF₃ produced in (b) to produce a product comprising at least compound selected from CF₃CH═CHF and CF₃CH═CF₂; and (d) recovering at least one compound selected from the group consisting of CF₃CH═CHF and CF₃CH═CF₂ from the product produced in (c).

Embodiment G2. The process of Embodiment G1 wherein the halopropene reactant is contacted with HF in a pre-reactor.

Embodiment G3. The process of Embodiment G1 wherein the reaction of (b) is conducted in a reaction zone at a temperature of from about 100° C. to about 350° C. containing a hydrogenation catalyst.

Embodiment G4. The process of Embodiment G1 wherein the reaction of (c) is conducted in the absence of a catalyst at a temperature of from about 350° C. to about 900° C.

Embodiment G5. The process of Embodiment G1 wherein the reaction of (c) is conducted in a reaction zone containing a dehydrofluorination catalyst at a temperature of from about 200° C. to about 500° C.

Embodiment G6. The process of Embodiment G1 wherein the amount of modifier metals relative to the total amount of chromium and modifier metals in the catalyst composition is from about 0.5 atom % to about 5 atom %.

Embodiment G7. The process of Embodiment G1 wherein the catalyst composition further comprises fluorine as an essential constituent element.

Embodiment G8. The process of Embodiment G1 wherein the catalyst composition comprises alpha-chromium oxide, metallic gold and metallic silver.

Embodiment G9. The process of Embodiment G1 wherein the catalyst composition comprises alpha-chromium oxide, metallic gold and palladium.

Embodiment G10. The process of Embodiment G1 wherein the catalyst composition comprises alpha-chromium oxide, metallic silver and palladium.

Embodiment G11. The process of Embodiment G1 wherein the catalyst composition comprises alpha-chromium oxide, metallic gold, metallic silver and palladium.

Embodiment G12. The process of Embodiment G1 wherein the catalyst composition comprises silver and gold and the mole ratio of silver to gold is from about 10:1 to about 1:10.

Embodiment G13. The process of Embodiment G1 wherein the catalyst composition comprises silver and palladium and the mole ratio of silver to palladium is from about 10:1 to about 1:10.

Embodiment G14. The process of Embodiment G1 wherein the catalyst composition comprises palladium and gold and the mole ratio of palladium to gold is from about 10:1 to about 1:10

### Examples

Reference is made to Examples A1-A5 in Invention Category A above for the chlorofluorination of CFC-1213xa.

Examination of the data in Table A1 above shows that the fluorine content of the starting CFC-1213xa is increased to produce CFC-1215xc and HCFC-226da that contain a higher fluorine content than the starting material by using the catalysts of this invention. The CFC-1215xc and HCFC-226da produced above may be hydrogenated to produce HFC-245fa and HFC-236fa, respectively, in a manner analogous to the teachings of U.S. Pat. No. 5,136,113. For example, the HFC-245fa and HFC-236fa may be dehydrofluorinated to HFC-1234ze and HFC-1225zc, respectively, in accordance with the teachings described in U.S. Pat. No. 6,369,284. The HFC-1234ze and HFC-1225zc may be recovered individually or as mixtures of HFC-1234ze and HFC-1225zc by procedures known to the art.

The reactor, distillation columns, and their associated feed lines, effluent lines, and associated units used in applying the processes described in Invention Categories A through G should be constructed of materials resistant to hydrogen fluoride and hydrogen chloride. Typical materials of construction, well-known to the fluorination art, include stainless steels, in particular of the austenitic type, the well-known high nickel alloys, such as Monel™ nickel-gold alloys, Hastelloy™ nickel-based alloys and, Inconel™ nickel-chromium alloys, and gold-clad steel.

Without further elaboration, it is believed that one skilled in the art can, using the descriptions herein (including the description in Invention Categories A through G above), utilize the present invention to its fullest extent. The specific embodiments are, therefore, to be construed as merely illustrative, and do not constrain the remainder of the disclosure in any way whatsoever.

1. A catalyst composition, comprising chromium, oxygen, and at least two modifier metals selected from the group consisting of gold, silver, and palladium, as essential constituent elements thereof, wherein the total amount of modifier metals is from about 0.05 atom % to about 10 atom % based on the total amount of chromium and modifier metals in the catalyst composition.

2. The catalyst composition of claim 1 further comprising fluorine as an essential constituent element.

3. The catalyst composition of claim 1 comprising gold and silver in a mole ratio of from about 10:1 to about 1:10.

4. The catalyst composition of claim 1 comprising gold and palladium in a mole ratio of from about 10:1 to about 1:10.

5. The catalyst composition of claim 1 comprising silver and palladium in a mole ratio of from about 10:1 to about 1:10.

6. The catalyst composition of claim 1, comprising particles of modifier metals supported on a chromium oxide support.

7. A process for changing the fluorine distribution in a hydrocarbon or halogenated hydrocarbon in the presence of a catalyst, characterized by using the catalyst composition of claim 1 as the catalyst.

8. The process of claim 7 wherein the fluorine content of a halogenated hydrocarbon compound or an unsaturated hydrocarbon compound is increased by reacting said compound with hydrogen fluoride in the vapor phase in the presence of said catalyst composition.

9. The process of claim 7 wherein the fluorine content of a halogenated hydrocarbon compound or a hydrocarbon compound is increased by reacting said compound with HF and Cl₂ in the presence of said catalyst composition.

10. The process of claim 7 wherein the fluorine distribution in a halogenated hydrocarbon compound is changed by isomerizing said halogenated hydrocarbon compound in the presence of said catalyst composition.

11. The process of claim 7 wherein the fluorine distribution in a halogenated hydrocarbon compound is changed by disproportionating said halogenated hydrocarbon compound in the presence of said catalyst composition.

12. The process of claim 7 wherein the fluorine content of a halogenated hydrocarbon compound is decreased by dehydrofluorinating said halogenated hydrocarbon compound in the presence of said catalyst composition.

13. The process of claim 7 wherein the fluorine content of a halogenated hydrocarbon compound is decreased by reacting said halogenated hydrocarbon compound with HCl in the vapor phase the presence of said catalyst composition.

14. A method for preparing the catalyst composition of claim 1, comprising:
(a) co-precipitating a solid by adding ammonium hydroxide to an aqueous solution of a soluble modifier metal salts and a soluble chromium salt that contains at least three moles of nitrate per mole of chromium in the solution and has a modifier metal content of from about 0.05 atom % to about 10 atom % of the total content of modifier metal and chromium in the solution, to form an aqueous mixture containing co-precipitated solid;
(b) drying said co-precipitated solid formed in (a); and
(c) calcining said dried solid formed in (b) in an atmosphere containing at least 10% oxygen by volume.

15. The method of claim 14 further comprising treating a calcined solid formed in (c) with a fluorinating agent to form a catalyst composition comprising chromium, oxygen, modifier metals, and fluorine as essential elements.

16. A method for preparing the catalyst composition of claim 1, comprising:
(a) impregnating solid chromium oxide with a solution of a soluble modifier metal salts;
(b) drying the impregnated chromium oxide prepared in (a); and
(c) calcining the dried solid.

17. The method of claim 16 further comprising treating a calcined solid formed in (c) with a fluorinating agent to form a catalyst composition comprising chromium, oxygen, modifier metals, and fluorine as essential elements.

18. A method for preparing the catalyst composition of claim 1, comprising:
mixing multiple compositions, each comprising chromium, oxygen, and at least one modifier metal.

19. The method of claim 18 further comprising treating the mixture of multiple compositions with a fluorinating agent to form a catalyst composition comprising chromium, oxygen, modifier metals, and fluorine as essential elements.

20. A process for making CF₃CH₂CHF₂ and CF₃CHFCH₂F, comprising:
(a) reacting HF, Cl₂, and at least one halopropene of the formula CX₃CCl═CClX, wherein each X is independently selected from the group consisting of F and Cl, to produce a product comprising CF₃CCl₂CClF₂ and CF₃CClFCCl₂F, wherein said CF₃CCl₂CClF₂ and CF₃CClFCCl₂F are produced in the presence of a catalyst composition of claim 1;
(b) reacting CF₃CCl₂CClF₂ and CF₃CClFCCl₂F produced in (a) with H₂, to produce a product comprising CF₃CH₂CHF₂ and CF₃CHFCH₂F; and
(c) recovering CF₃CH₂CHF₂ and CF₃CHFCH₂F from the product produced in (b).

21. A process for the manufacture of at least one compound selected from the group consisting of 1,3,3,3-tetrafluoropropene and 2,3,3,3-tetrafluoropropene, comprising:
(a) reacting hydrogen fluoride, chlorine, and at least one halopropene of the formula CX₃CCl═CClX, wherein each X is independently selected from the group consisting of F and Cl, to produce a product comprising CF₃CCl₂CClF₂ and CF₃CClFCCl₂F, wherein said CF₃CCl₂CClF₂ and CF₃CClFCCl₂F are produced in the presence of a catalyst composition of claim 1;
(b) reacting CF₃CCl₂CClF₂ and CF₃CClFCCl₂F produced in (a) with hydrogen to produce a product comprising CF₃CH₂CHF₂ and CF₃CHFCH₂F;
(c) dehydrofluorinating CF₃CH₂CHF₂ and CF₃CHFCH₂F produced in (b) to produce a product comprising CF₃CH═CHF and CF₃CF═CH₂; and
(d) recovering at least one compound selected from the group consisting of CF₃CH═CHF and CF₃CF═CH₂ from the product produced in (c).

22. A process for the manufacture of 1,1,1,3,3,3-hexafluoropropane and at least one compound selected from the group consisting of 1,1,1,2,3,3-hexafluoropropane and hexafluoropropene, comprising:
(a) reacting HF, Cl₂, and at least one halopropene of the formula CX₃CCl═CClX, wherein each X is independently selected from the group consisting of F and Cl, to produce a product comprising CF₃CCl₂CF₃ and CF₃CClFCClF₂, wherein said CF₃CCl₂CF₃ and CF₃CClFCClF₂ are produced in the presence of a catalyst composition of claim 1;
(b) reacting CF₃CCl₂CF₃ and CF₃CClFCClF₂ produced in (a) with hydrogen, optionally in the presence of HF, to produce a product comprising CF₃CH₂CF₃ and at least one compound selected from the group consisting of CHF₂CHFCF₃, CF₃CF═CF₂ and CF₃CFHCF₃; and
(c) recovering from the product produced in (b), CF₃CH₂CF₃ and at least one compound selected from the group consisting of CHF₂CHFCF₃, CF₃CF═CF₂ and CF₃CFHCF₃.

23. A process for the manufacture of at least one compound selected from the group consisting of 1,1,3,3,3-pentafluoropropene and 1,2,3,3,3-pentafluoropropene, comprising:
(a) reacting hydrogen fluoride, chlorine, and at least one halopropene of the formula CX₃CCl═CClX, wherein each X is independently selected from the group consisting of F and Cl, to produce a product comprising CF₃CCl₂CF₃ and CF₃CClFCClF₂, wherein said CF₃CCl₂CF₃ and CF₃CClFCClF₂ are produced in the presence of a catalyst composition of claim 1;
(b) reacting CF₃CCl₂CF₃ and CF₃CClFCClF₂ produced in (a) with hydrogen, optionally in the presence of hydrogen fluoride, to produce a product comprising CF₃CH₂CF₃ and CF₃CHFCHF₂;
(c) dehydrofluorinating CF₃CH₂CF₃ and CF₃CHFCHF₂ produced in (b) to produce a product comprising CF₃CH═CF₂ and CF₃CF═CHF; and
(d) recovering at least one compound selected from the group consisting of CF₃CH═CF₂ and CF₃CF═CHF from the product produced in (c).

24. A process for making at least one compound selected from CF₃CH₂CHF₂ and CF₃CH₂CF₃, comprising:
(a) reacting HF, and at least one halopropene of the formula CX₃CCl═CClX, wherein each X is independently selected from the group consisting of F and Cl, to produce a product comprising at least one compound selected from CF₃CCl═CF₂ and CF₃CHClCF₃, wherein said CF₃CCl═CF₂ and CF₃CHClCF₃ are produced in the presence of a catalyst composition of claim 1;
(b) reacting at least one compound selected from CF₃CCl═CF₂ and CF₃CHClCF₃ produced in (a) with H₂, optionally in the presence of HF, to produce a product comprising at least one compound selected from CF₃CH₂CHF₂ and CF₃CH₂CF₃; and
(c) recovering at least one compound selected from CF₃CH₂CHF₂ and CF₃CH₂CF₃ from the product produced in (b).

25. A process for the manufacture of at least one compound selected from the group consisting of 1,3,3,3-tetrafluoropropene and 1,1,3,3,3-pentafluoropropene, comprising:
(a) reacting HF, and at least one halopropene of the formula CX₃CCl═CClX, wherein each X is independently selected from the group consisting of F and Cl, to produce a product comprising at least compound selected from CF₃CCl═CF₂ and CF₃CHClCF₃, wherein said CF₃CCl═CF₂ and CF₃CHClCF₃ are produced in the presence of a catalyst composition of claim 1;
(b) reacting at least compound selected from CF₃CCl═CF₂ and CF₃CHClCF₃ produced in (a) with H₂, optionally in the presence of HF, to produce a product comprising at least compound selected from CF₃CH₂CHF₂ and CF₃CH₂CF₃; and
(c) dehydrofluorinating at least compound selected from CF₃CH₂CHF₂ and CF₃CH₂CF₃ produced in (b) to produce a product comprising at least compound selected from CF₃CH═CHF and CF₃CH═CF₂; and
(d) recovering at least one compound selected from the group consisting of CF₃CH═CHF and CF₃CH═CF₂ from the product produced in (c).
